(19) United States
(12) Patent Application Publication
(10) Pub. No.: US 2026/0200992 A1
(43) Pub. Date: Jul. 16, 2026

(54) A MOLECULAR TOOLKIT FOR HETEROLOGOUS PROTEIN SECRETION IN BACTEROIDES SPECIES

(71) Applicant: The Brd. of Trustees of the Univ. of Illinois, Urbana, IL (US)

(72) Inventors: Shannon Sirk, Urbana, IL (US); Yu-Hsuan Yeh, Urbana, IL (US)

(73) Assignee: The Brd. of Trustees of the Univ. of Illinois, Urbana, IL (US)

(21) Appl. No.: 19/136,006

(22) PCT Filed: Dec. 8, 2023
(86) PCT No.: PCT/US2023/083131

§ 371 Date: Jun. 5, 2025

Related U.S. Application Data
(60) Provisional application No. 63/431,236, filed on Dec. 8, 2022.

(51) Int. Cl. C07K 14/195; C07K 16/32; C12N 15/63; C12N 15/74

(52) U.S. Cl. CPC C07K 14/195; C07K 16/32; C12N 15/635; C12N 15/74; C07K 2317/24; C07K 2317/569; C07K 2317/622

(57) ABSTRACT

Provided herein are recombinant polynucleotides comprising a promoter, a ribosome binding site, a sequence encoding a secretion carrier, and a sequence encoding a heterologous protein. Also provided are polypeptides comprising a secretion carrier and a sequence encoding a heterologous protein. Methods of exporting heterologous polypeptides from cells are additionally provided.

### RELATED APPLICATIONS

This application claims priority to U.S. Provisional Patent Application Ser. No. 63/431,236, filed Dec. 8, 2022, the entire disclosures of which are hereby incorporated herein by reference.

### BACKGROUND

Bacteroides species, one of the most abundant and prevalent bacterial populations in the human gut, are capable of long-term, stable colonization of the gastrointestinal tract, making them a promising chassis for developing long-term interventions for chronic diseases. However, a lack of efficient heterologous protein secretion tools prevents their use as engineered, on-site delivery vehicles for protein-based biologic drugs or disease-responsive reporters.

When engineering bacteria for therapeutic or diagnostic purposes in which protein-based products must function in the extracellular space, the ability of the microbial chassis to secrete heterologous cargo is a key selection criterion. While the Bacteroides genus represents an attractive collection of target species for this purpose, all Bacteroides species are Gram-negative, which presents technical challenges for efficient protein secretion. Unlike Gram-positive bacteria, which have a single lipid membrane and readily secrete heterologous cargo through both the general secretion pathway (Sec) and twin-arginine translocation (Tat) pathway as long as the target protein is fused to an appropriate signal peptide (SP), protein secretion from double-membraned Gram-negative bacteria is more complex and requires additional cellular machinery. Secretion systems have been identified in Gram-negative bacteria, however, these secretion systems are either poorly conserved or completely absent from all Bacteroides species studied to date. Methods of using Bacteroides species as a platform for engineered living therapeutics are needed in the art.

### SUMMARY

Provided herein are recombinant polynucleotides comprising a promoter, a ribosome binding site, a sequence encoding a secretion carrier, and a sequence encoding a heterologous protein. The promoter can be a Bacteroides promoter. The promoter can be an inducible promoter or a constitutive promoter. The ribosome binding site can be derived from BT1311, or can be RBS8 or A21 RBS. The secretion carrier can be a truncated membrane-associated Bacteroides lipoprotein or a full-length membrane-associated Bacteroides lipoprotein. The recombinant polynucleotide can encode a secretion carrier comprising (from N terminus to a C terminus) a positively charged region of about 3 to 9 amino acids, a hydrophobic region of about 13-34 amino acids, and a lipoprotein secretion sequence. The charged region can comprise a polypeptide as set forth in SEQ ID NO:123 or SEQ ID NO:124, and the lipoprotein secretion sequence can comprise a polypeptide as set forth in SEQ ID NO:125. The heterologous protein can be a therapeutic protein that is an antibody or specific binding fragment thereof, a cytokine, or a growth factor. The antibody or specific binding fragment thereof can be a scFv, Fab, Fab′, Fv, F(ab′)₂, a minibody, a diabody, a triabody, a tetrabody, a tandem di-scFv, a tandem tri-scFv, an immunoglobulin single variable domain (ISV), a V_{HH}, a humanized V_{HH}, a camelized V_{H}, a single domain antibody, a domain antibody, or a dAb. 12. A recombinant polynucleotide can further comprise a linker or cleavage site positioned between or within the secretion carrier and the heterologous protein.

Another aspect provides a recombinant polypeptide comprising (i) a secretion carrier comprising a positively charged region of about 3 to about 9 amino acids, a hydrophobic region of about 13 to 34 amino acids, and a lipoprotein export sequence; and (ii) a heterologous polypeptide. The secretion carrier can be a truncated membrane-associated Bacteroides lipoprotein or a full-length membrane-associated Bacteroides lipoprotein. The heterologous protein can be a therapeutic protein comprising an antibody or specific binding fragment thereof, a cytokine, or a growth factor. The positively charged region can be set forth in SEQ ID NO:123 or SEQ ID NO:124, and the lipoprotein secretion sequence can be set forth in SEQ ID NO:125. The antibody or specific binding fragment thereof can be a scFv, Fab, Fab′, Fv, F(ab′)₂, a minibody, a diabody, a triabody, a tetrabody, a tandem di-scFv, a tandem tri-scFv, an immunoglobulin single variable domain (ISV), a V_{HH}, a humanized V_{HH}, a camelized V_{H}, a single domain antibody, a domain antibody, or a dAb.

Another aspect provides a vector comprising any of the polynucleotides described herein, a recombinant cell comprising any of the polynucleotides described herein. A recombinant cell can be a Bacteroides cell. The Bacteroides cell can be a B. thetaiotaomicron, B. ovatus, B. fragilis, B. vulgatus, B. distasonis or B. uniformis cell.

Yet another aspect provides a method of exporting a heterologous polypeptide from a cell comprising delivering the recombinant polynucleotides described herein to the cell. The heterologous polypeptide can be freely soluble in an extracellular space of the cell; bound to an external surface of an outer membrane vesicle (OMV); or held within an OMV lumen.

Even another aspect provides a method of treatment comprising administering the recombinant cells described herein to a subject. The subject can have an intestinal disorder. The intestinal disorder can be inflammatory bowel disease (IBD) or Crohn's disease. The recombinant cells can be administered orally or intrarectally.

### DETAILED DESCRIPTION OF THE DRAWINGS

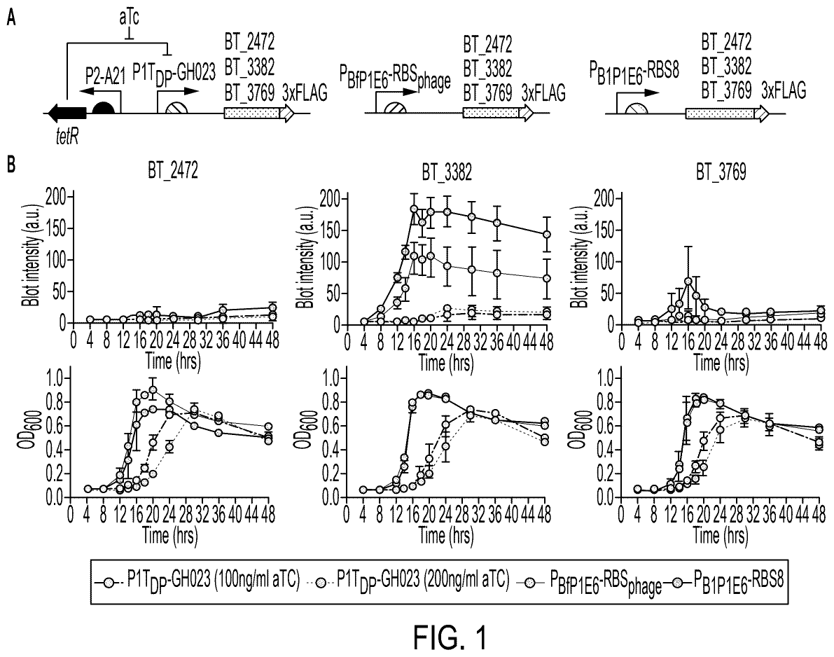

FIG. 1 panels A-B: Characterization and optimization of protein secretion in B. theta. Panel (A) shows design of genetic constructs for protein expression and secretion. Panel (B) shows protein secretion (top row) and growth (bottom row) of B. theta expressing BT_2472, BT_3382, and BT_3769 measured from supernatant samples taken every 4 hrs for 48 hrs. For each sample, protein secretion levels were measured by dot blot and bacterial growth was measured by optical density at 600 nm (OD₆₀₀). For strains using the P1T_{DP}-GH023 promoter, the inducer aTc (100 or 200 ng/ml) was included in the medium at the time of inoculation. Error bars represent standard deviation of three biological replicates. a.u., arbitrary units; aTC, anhydrotetracycline.

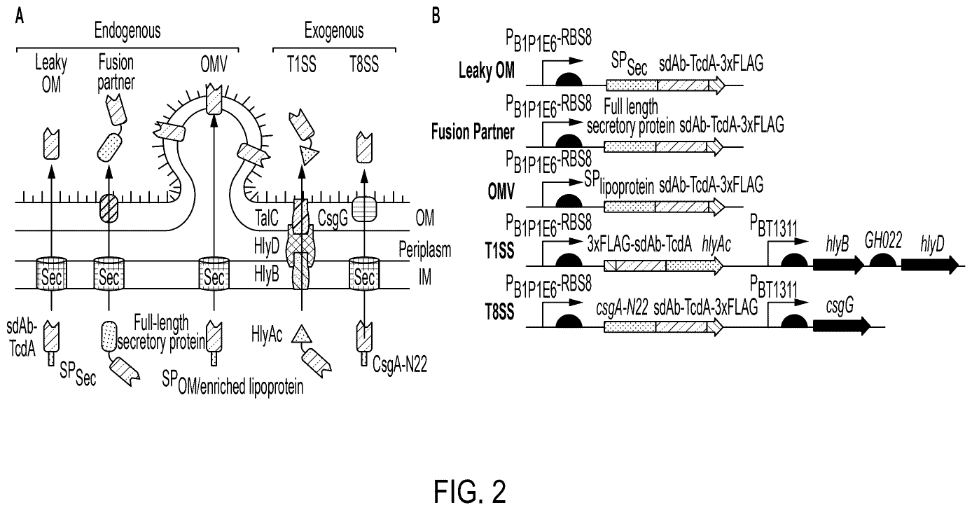

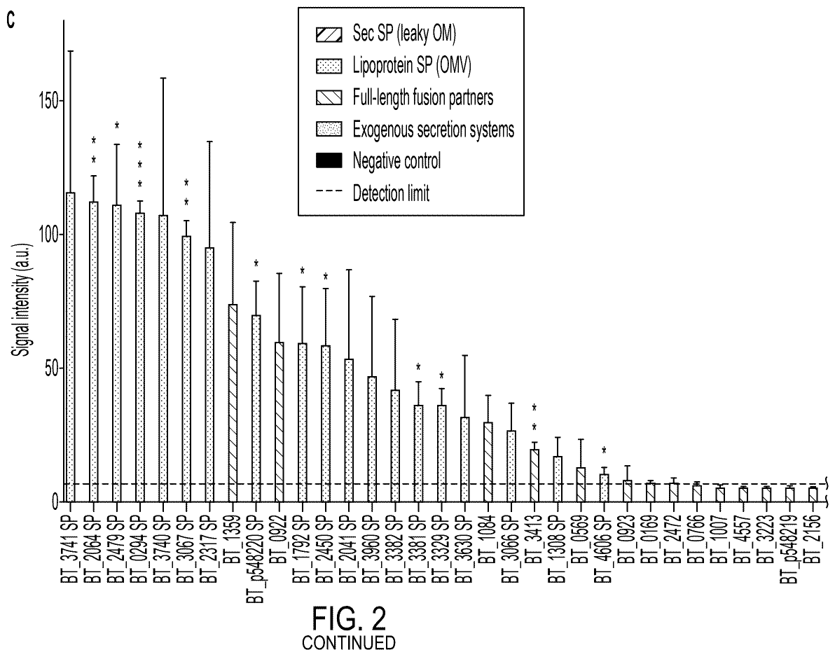

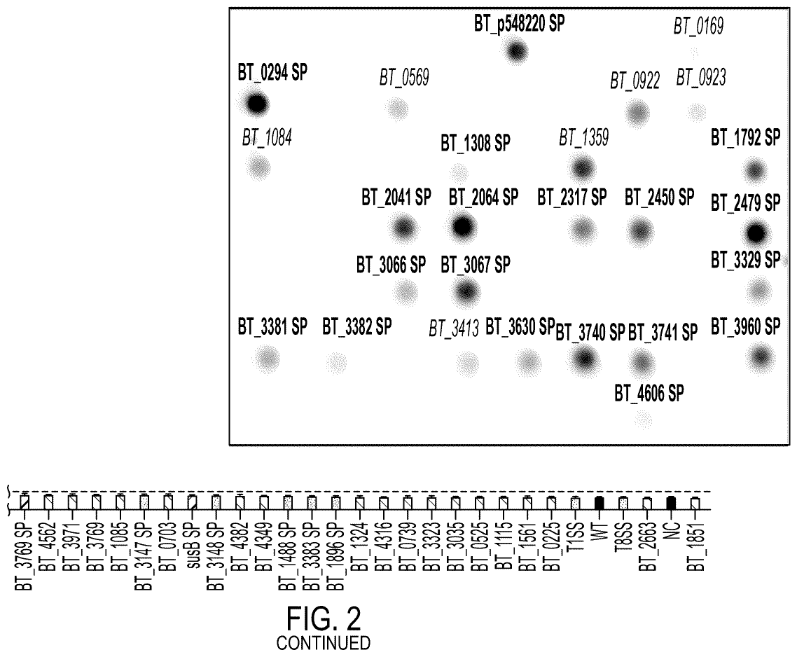

FIG. 2 Panels A-C: Large scale screen of candidate secretion carriers in B. theta. (A) Schematic representation of secretion strategies explored in this study. (B) Design of genetic constructs for secretion carrier screening in B. theta. (C) Relative levels of secretion of sdAb-TcdA in culture supernatants of B. theta harboring sixty different expression/secretion constructs, measured by dot blot. Inset shows representative dot blot with effective secretion carriers (above detection limit) labeled. Detection limit (dotted line) was set at the signal intensity of the faintest dot visible by unaided eye on the membrane, which is ~7 arbitrary units (a.u.). Error bars represent one standard deviation of triplicate biological samples. *p<0.05, **p<0.01, ***p<0.001. WT, wild-type B. theta; NC, negative control B. theta expressing sdAb-TcdA with no secretion carrier fusion.

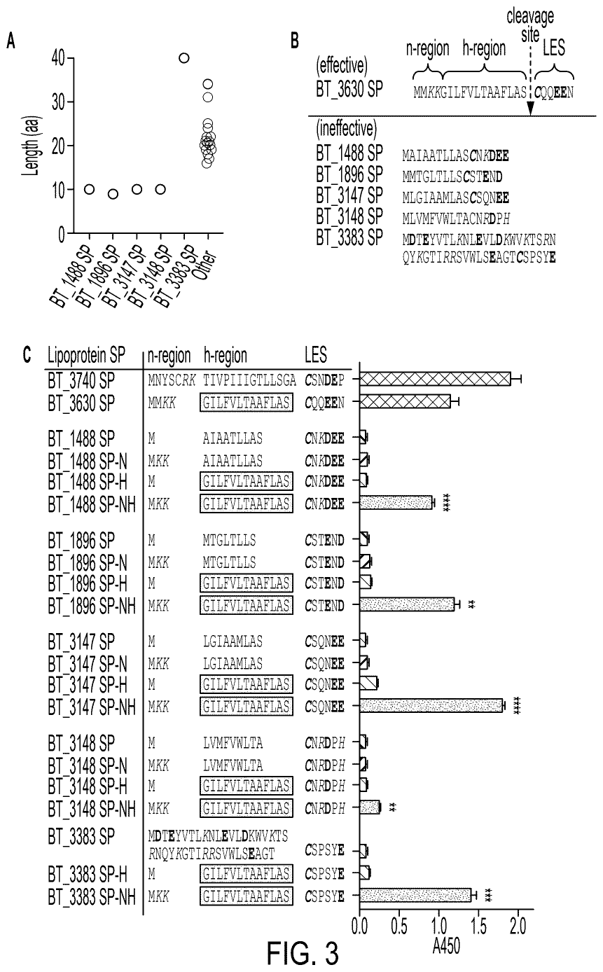

FIG. 3 Panels A-C: Rational engineering enables ineffective lipoprotein SPs to secrete sdAb-TcdA. (A) Comparison of the length of four ineffective (gray circles) and nineteen effective (green circles) lipoprotein SP sequences. (B) Comparison of amino acid sequences of the five ineffective lipoprotein SPs with a prototypical effective sequence. Positively charged residues are colored blue, negatively charged residues are colored red, and cysteine residue cleavage sites are colored green. MMKKGILFVLTAAFLASCQQEEN is SEQ ID NO:190; MAIATLLASCNKDEE is SEQ ID NO:19 1; MMTGLTLLSCSTEND is SEQ ID NO:192; MLGIAAMLASCSQNEE is SEQ ID NO:193; MLVMFVWLTACNRDPH is SEQ ID NO:194; MDTEYVTLKNLEVLDKVVKTSRNQYKGTIRRSVWLSEAGTCSPSYE is SEQ ID NO:195. (C) Secretion of sdAb-TcdA driven by native (SP) and domain-swapped (SP-N, SP-H, and SP-NH) ineffective lipoprotein SPs, measured by ELISA. BT_3630 SP-sdAb-TcdA and BT_3740 SP-sdAb-TcdA are included as positive controls. Residue coloring is the same as for (B), and hydrophobic regions are boxed in grey. **p<0.01, ***p<0.001, ****p<0.0001. MNYSCRK is SEQ ID NO:152; MMKK is SEQ ID NO:151; TIVPIIIGTLLSGA is SEQ ID NO:147; GILFVLTAAFLAS is SEQ ID NO:146; AIAATLLAS is SEQ ID NO:186; MTGLTLLS is SEQ ID NO:187; LGIAAMLAS is SEQ ID NO:188; LVMFVWLTA is SEQ ID NO:189; MDTEYVTLKNLEVLDKWVKTSRNQUKGTIRRS WWLSEAGT is SEQ ID NO:180; CSNDEP is SEQ ID NO:156; CQQEEN is SEQ ID NO:155; CNKDEE is SEQ ID NO:181; CSTEND is SEQ ID NO:182; CSQNEE is SEQ ID NO:183; CNRDPH is SEQ ID NO:184; CSPSYE is SEQ ID NO:185.

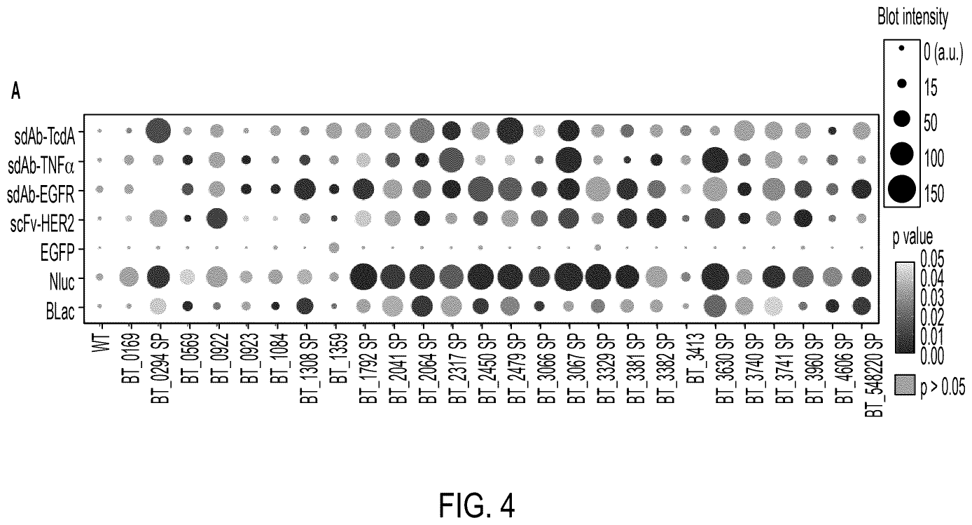

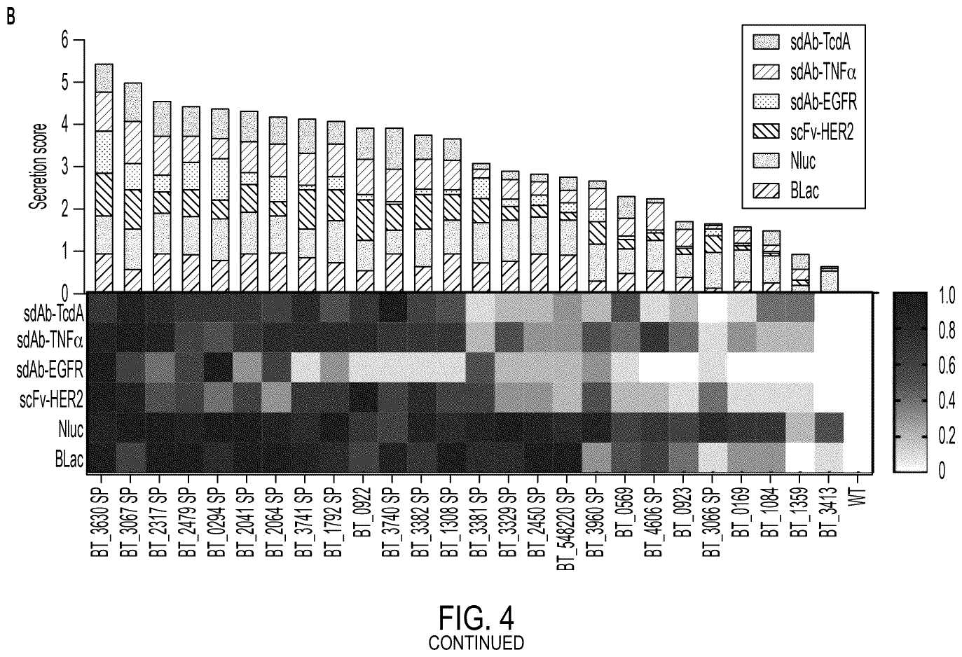

FIG. 4 Panels A-B: B. theta secretion carriers function across multiple heterologous proteins. (A) Relative levels of antibody fragments and reporter proteins secreted into culture supernatant by B. theta secretion carriers. Bubble size corresponds to average blot intensity of triplicate experiments with p<0.05 indicated by the blue color scale and p>0.05 shown in gray. Significance was determined using unpaired two-tailed Welch's t test. (B) Functional assays of antibody fragments and reporter proteins secreted into culture supernatant by B. theta secretion carriers. Binding of antibody fragments (sdAbs and scFv) to their respective targets was determined by ELISA. Enzymatic activity of reporter proteins Nluc and BLac was determined by bioluminescence assay and colorimetric assay, respectively. Following log transformation of luminescence data, all functional assay readouts were converted to values between zero and one by cargo-wise min-max normalization. “Secretion score” represents sum of normalized readouts of all six cargo proteins for each secretion carrier.

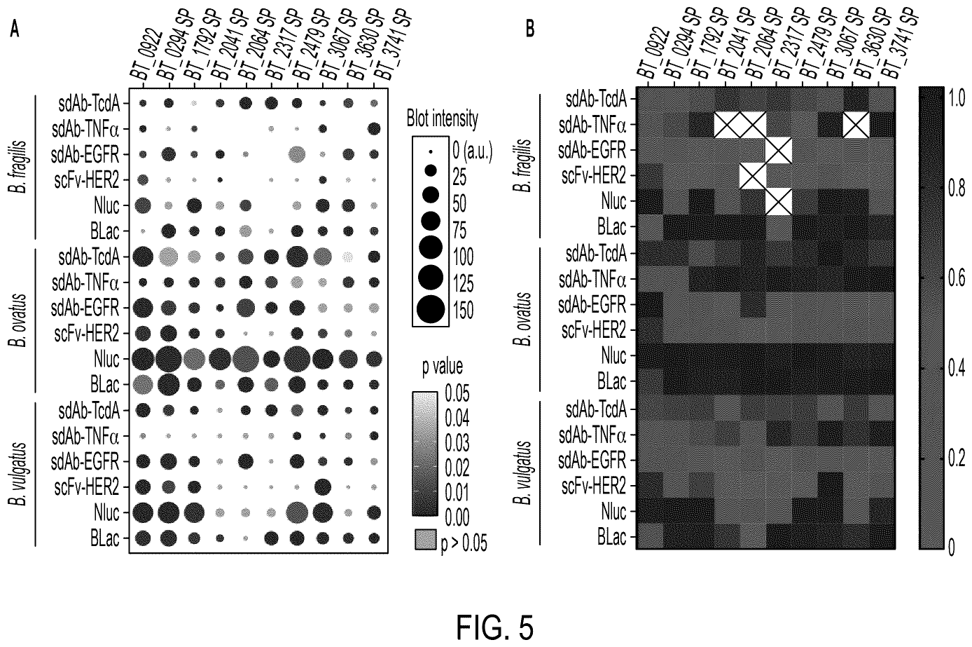

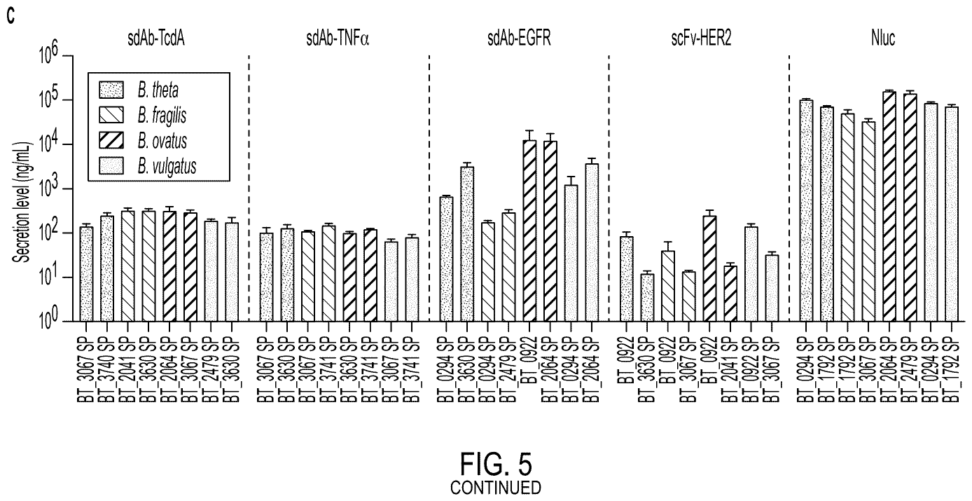

FIG. 5 Panels A-C: B. theta secretion carriers mediate export of diverse, functional cargo from multiple Bacteroides species. (A) Relative levels of six cargo proteins detected in the culture supernatants of three Bacteroides species, driven by each of the ten highest performing native B. theta secretion carriers. Bubble size corresponds to average blot intensity of triplicate experiments with p<0.05 indicated by the blue color scale and p>0.05 shown in gray. Significance was determined using unpaired two-tailed Welch's t test. (B) Functional assays of antibody fragments and reporter proteins secreted into culture supernatant by secretion carriers. Binding of antibody fragments (sdAb-TcdA, sdAb-TNFα, sdAb-EGFR, and scFv-HER2) to their respective targets was determined by ELISA. Enzymatic activity of reporter proteins (Nluc and BLac) was determined by bioluminescence assay and colorimetric assay, respectively. Following log transformation of luminescence data, all functional assay readouts were converted to values between zero and one by cargo-wise min-max normalization. (C) Quantification of protein secretion titers mediated by the two secretion carriers that yielded the highest functional protein levels of each cargo in each species. Error bars represent the standard deviation of triplicate experiments.

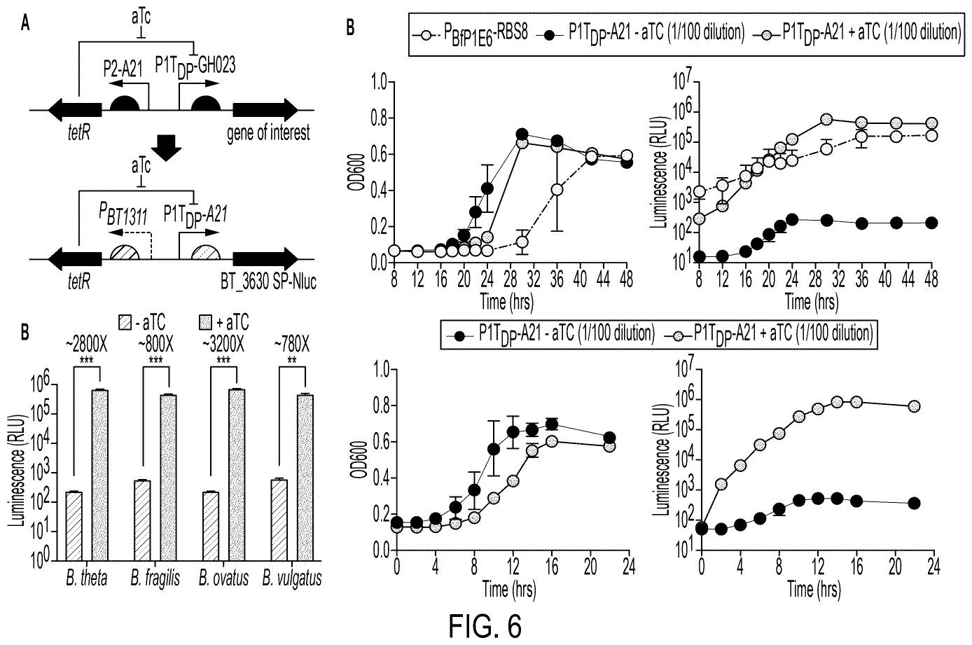

FIG. 6 Panels A-C: Development of a strong, aTc-inducible expression cassette for enhanced control of protein secretion across multiple Bacteroides species. (A) Low-activity promoter and RBS sequences in the original P2-A21-tetR-P1T_{DP}-GH023 inducible expression cassette (top) (20) were replaced with high-activity variants to generate the modified P_{BT1311}-tetR-P1T_{DP}-A21 expression cassette (bottom). (B) Modified inducible expression cassette drives expression of Nluc reporter at levels similar to high-level constitutive promoter P_{BfP1E6}-RBS8 in induction cultures diluted at 1:100 (top) or 1:10 (bottom). (C) Modified inducible system mediates tightly controlled protein expression across multiple Bacteroides species. For all experiments, luminescence measurements were obtained from clarified culture supernatant and thus represent the secreted fraction of total expressed Nluc. Nluc secretion in this study was mediated by the highly active secretion carrier BT_3630 SP. **p<0.01, ***p<0.001, ****p<0.0001.

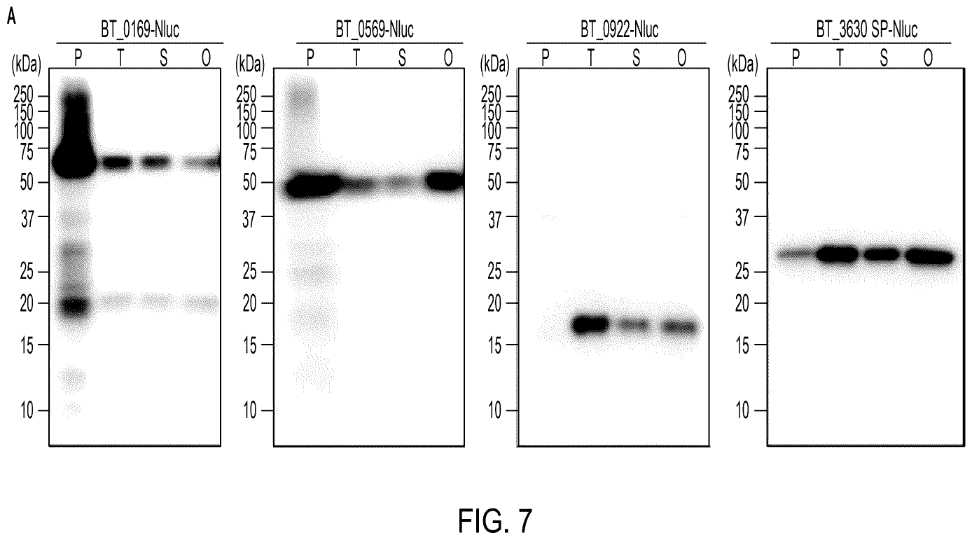

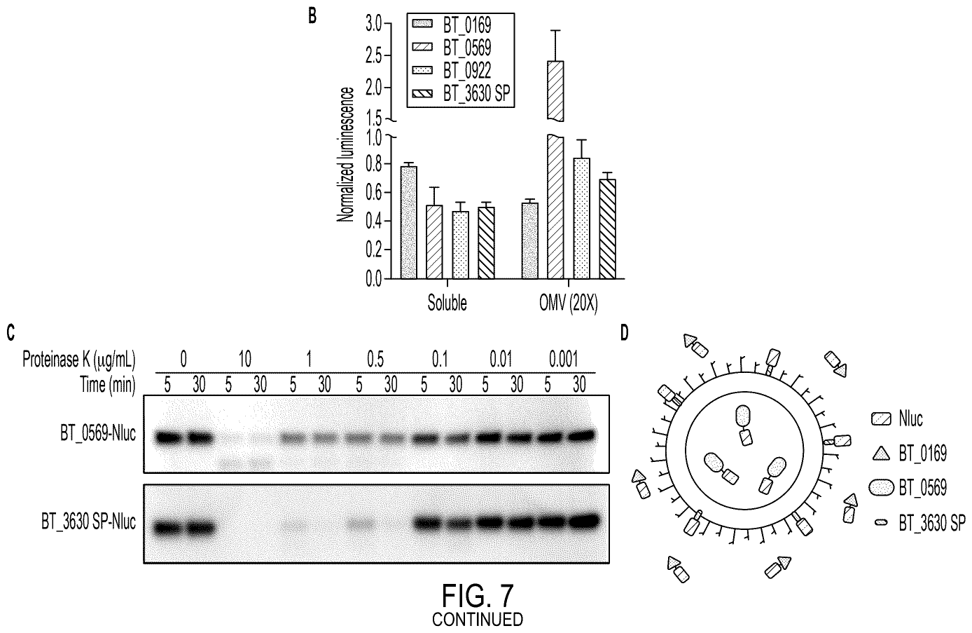

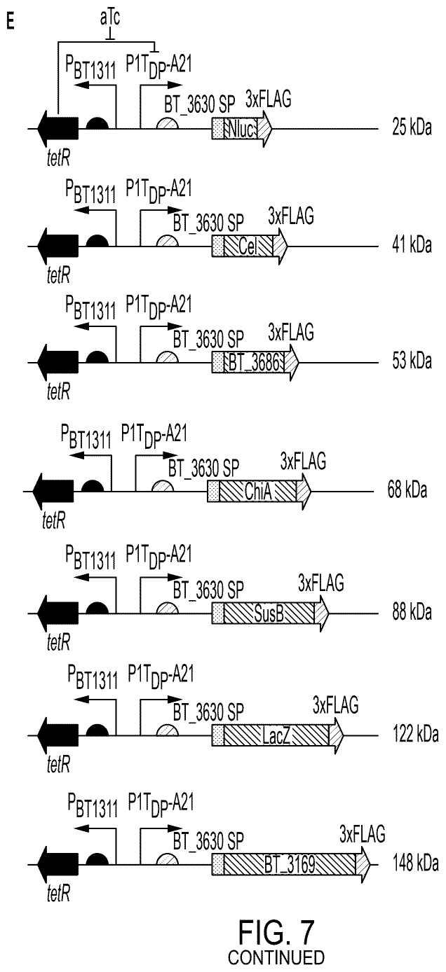

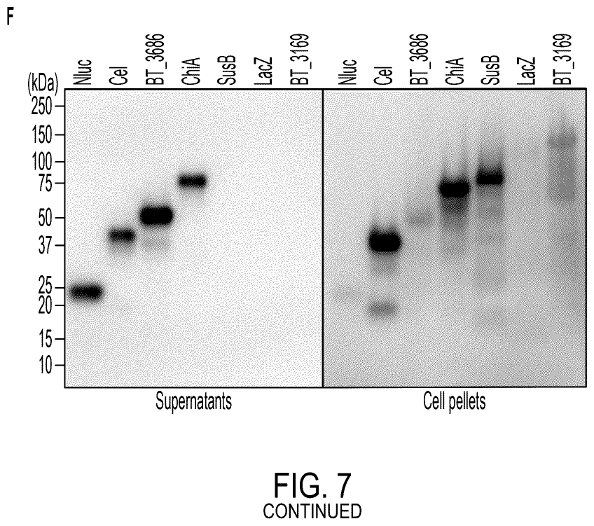

FIG. 7 Panels A-F; Characterization of the post-secretion extracellular fate and size limit of cargo proteins for secretion carriers. (A) Western blot analysis of Nluc abundance in different fractions of B. theta liquid cultures expressing four carrier-Nluc constructs. (P) cell pellet, (T) total supernatant, (S) soluble fraction of total supernatant, and (O) OMV fraction of total supernatant. (B) The enzymatic activity of secreted Nluc in soluble and OMV fractions, measured by luminescence assay. To normalize the efficiency difference of four secretion carriers, the luminescence in soluble and OMV fractions were divided by the luminescence in total supernatants to calculate the relative abundance of secreted Nluc in soluble and OMV fractions. (C) Western blot analysis of proteinase K assay of OMV fractions from B. theta cultures expressing BT_0569-Nluc (Sec SP; predicted localization to OMV lumen) and BT_3630-Nluc (lipoprotein SP; predicted localization to OMV surface). (D) Schematic representation of post-secretion extracellular fate of Nluc mediated by BT_0169, BT_0569, and BT_3630 SP. (E) Set of seven expression constructs generated to test the ability of BT_3630 SP to mediate secretion of different sized protein cargo to the outer surface of OMVs in B. theta. The molecular weight of each protein is shown on the right. (F) Western blot analysis of liquid culture supernatants and cell pellets from B. theta expressing seven proteins of varying size fused to BT_3630 SP.

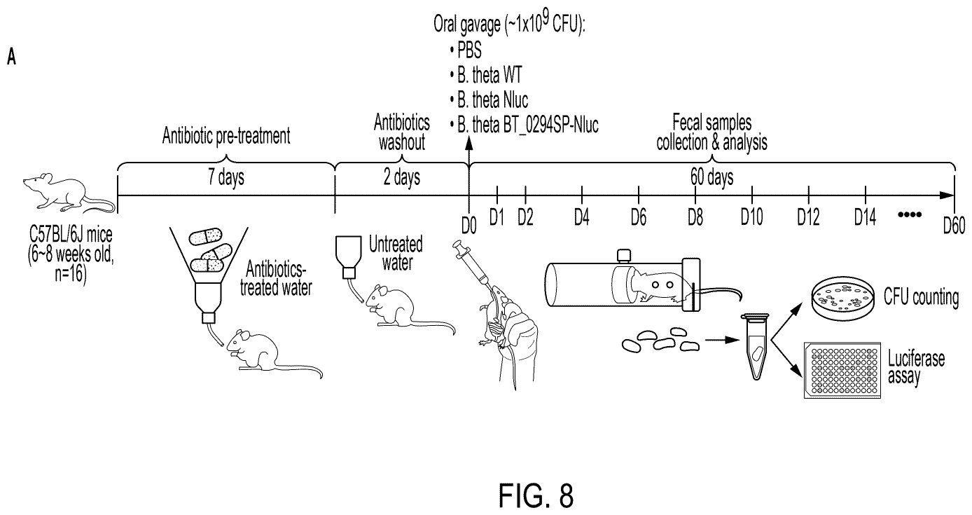

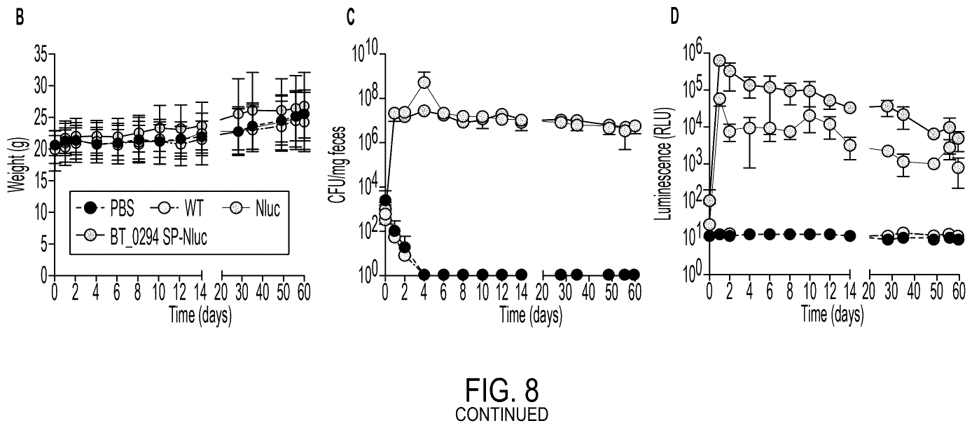

FIG. 8 Panels A-D: Direct intestinal delivery of heterologous protein cargo by B. theta in mice. (A) Design of in vivo experiments. Mice were monitored, and fecal samples were collected and analyzed for two months following inoculation. (B) The weight of mice in all groups increased similarly over time, indicating no adverse health effects. (C) Engineered B. theta strains persisted at high levels in the mouse intestine, as determined by fecal CFU counts. (D) The functionality of intestinally delivered protein cargo (Nluc) persisted over time, as determined by luminescence measurements of fecal homogenates.

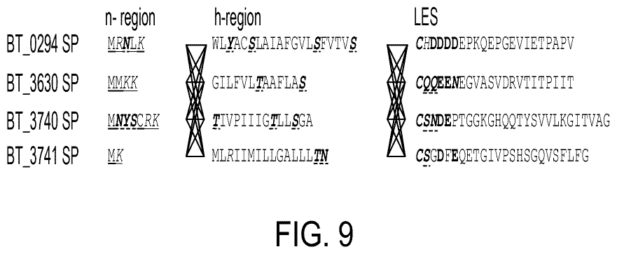

FIG. 9 shows lipoprotein secretion proteins with diverse n-, h-, and LES regions that were used for domain shuffling. MRNLK is SEQ ID NO:150; MMKK is SEQ ID NO:151; MNYSCRK is SEQ ID NO:152; WLYACSLAIAFGVLSFVTVS is SEQ ID NO:139; GILFVTAAAFLAS is SEQ ID NO:146; TIVPIIIGTLLSGA is SEQ ID NO:147; MLRIIMILLGALLLTN is SEQ ID NO:145; CHDDDDEPKQEPGEVIETPAPV is SEQ ID NO:170; CQQEENEGVASVDRVTITPIIT is SEQ ID NO:171; CSGDFEQETGIVPS HSGQVSFLFG is SEQ ID NO:173.

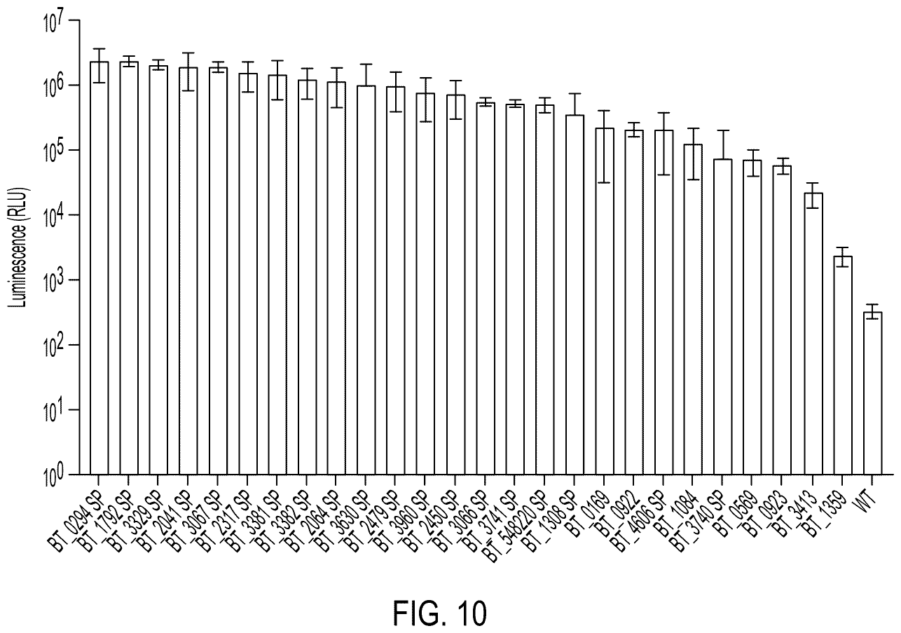

FIG. 10 shows Nluc secretion efficiency of various secretion carriers.

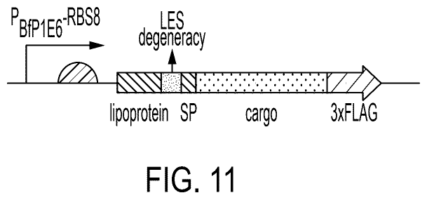

FIG. 11 shows the design of a lipoprotein SP backbone.

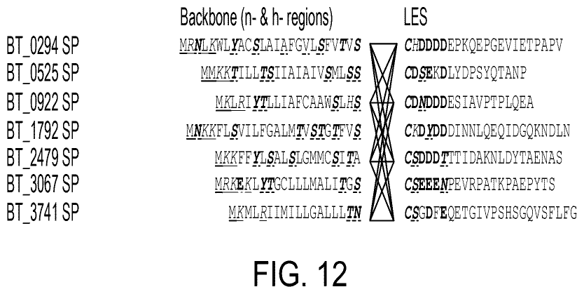

FIG. 12 shows the sequences lipoprotein SP backbones and LES regions. MRNLKWLYACSLAIAFGVLSFVTVS is SEQ ID NO:163, MMKKTILLTSIIAIAIVSMLSS is SEQ ID NO:164, MKLRIYTLLIAFCAAWSLHS is SEQ ID NO:165, MNKKFLSVILFGALMTVSTGTFVS is SEQ ID NO:166, MKKFFYLSALSLGMMCSITA is SEQ ID NO:167, MRKEKLYTGCLLLMALITGS is SEQ ID NO:168, and MKMLRIIMILLGALLLTN is SEQ ID NO:169; CHDDDDEPKQEPGEVIETPAPV is SEQ ID NO:170; CSGDFEQETGIVPS HSGQVSFLFG is SEQ ID NO:173; CDSEKDLYDPSYQTANP is SEQ ID NO:174; CDNDDDESIAVPTPLQEA is SEQ ID NO:175; CKDYDDDINNLQEQIDGQKNDLN is SEQ ID NO:176; CSDDDTTTIDAKNLDYTAENAS is SEQ ID NO:177; CSEEENPEVRPATKPAEPYTS is SEQ ID NO:178.

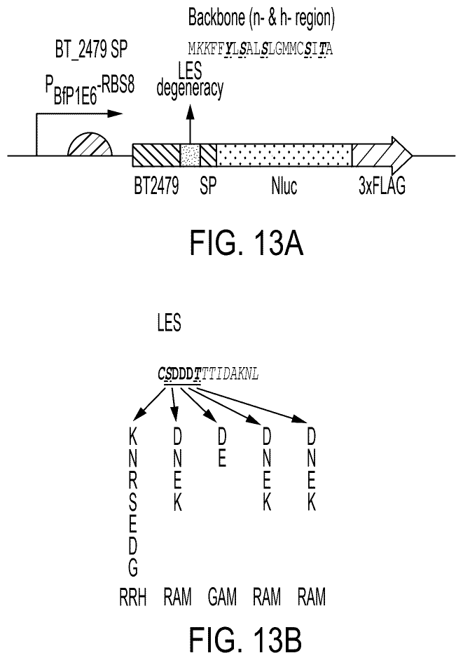

FIG. 13A shows the BT2479 SP backbone (SEQ ID NO:167)

FIG. 13B shows tested LES regions (SEQ ID NO:179).

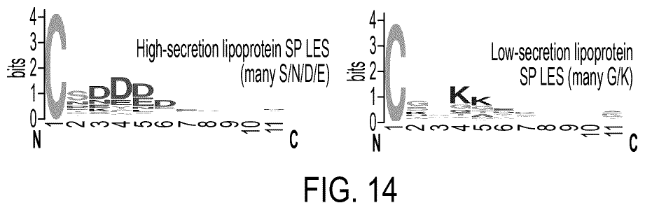

FIG. 14 shows the LES sequences of high efficiency secretion carriers low-efficiency secretion carriers.

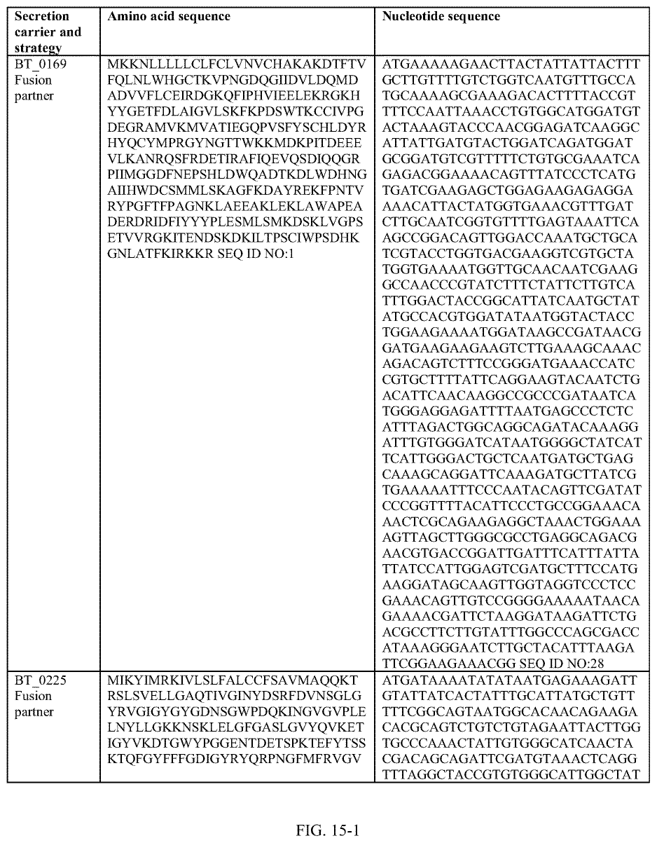

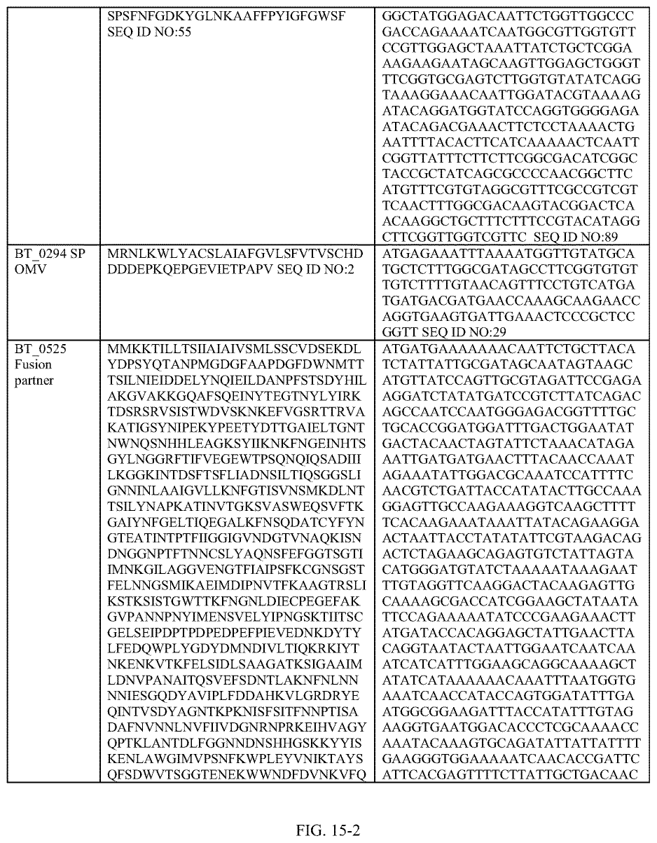

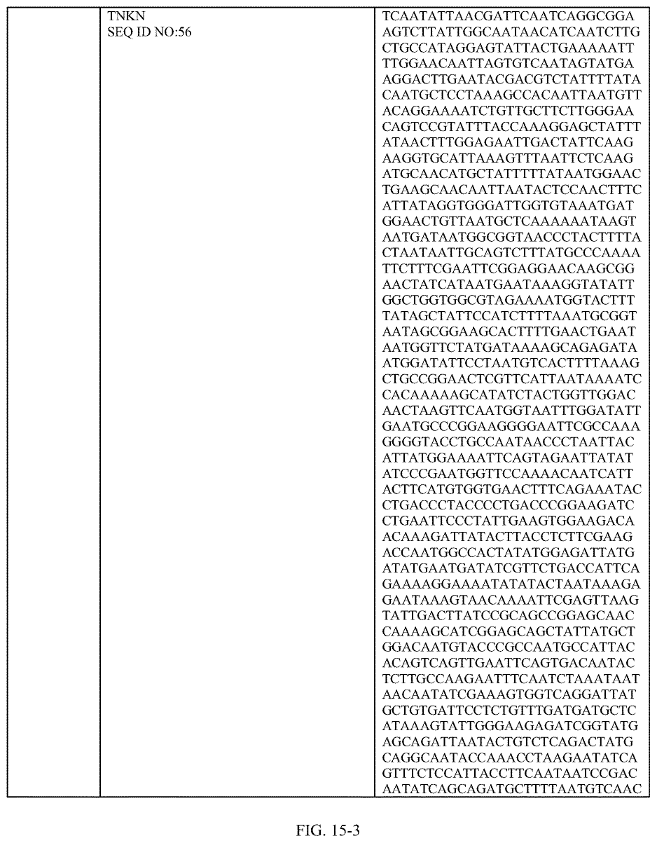

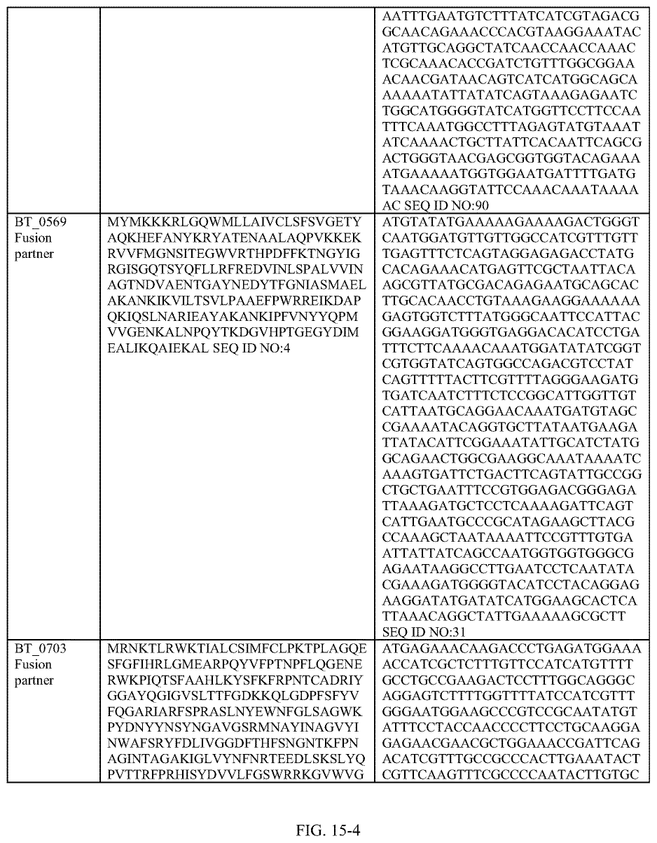

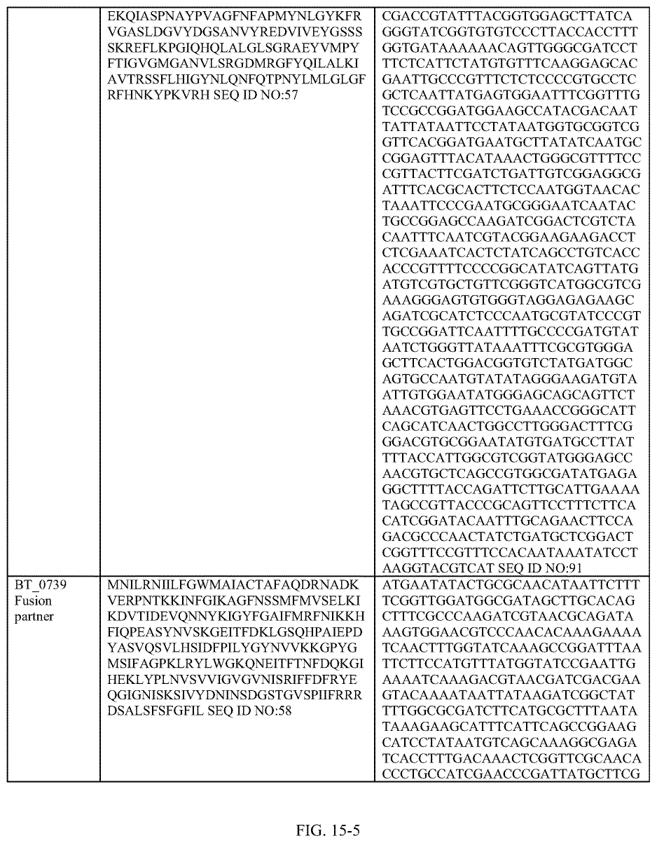

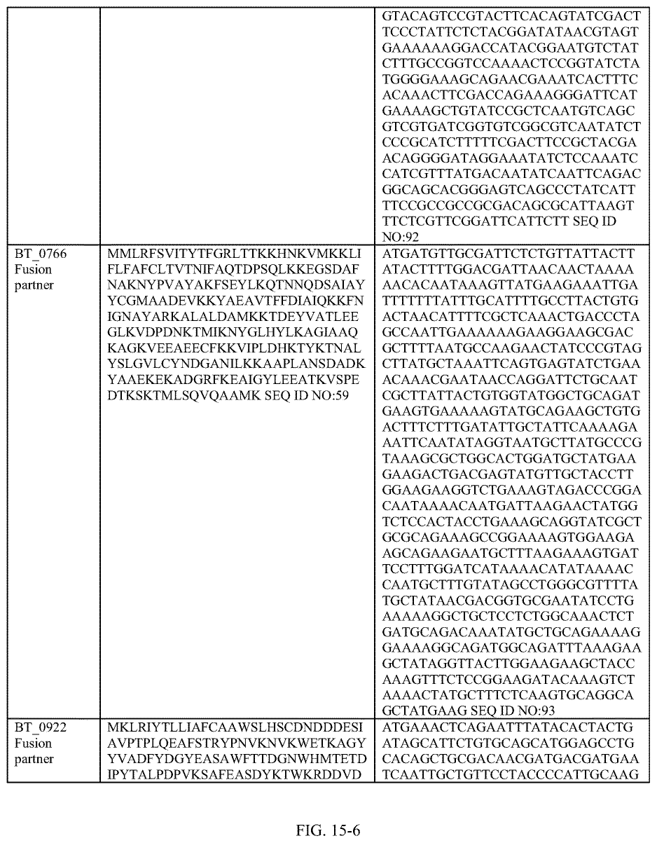

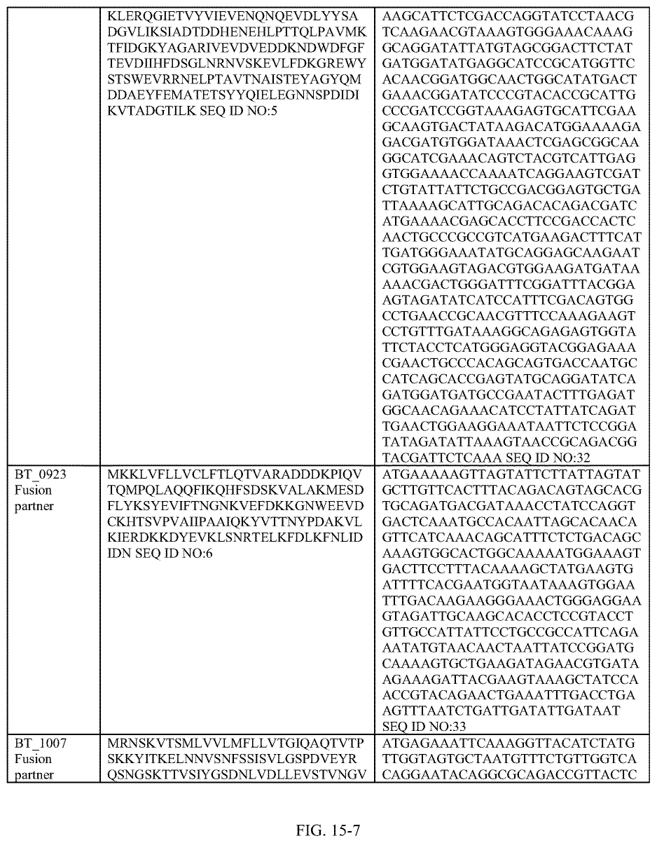

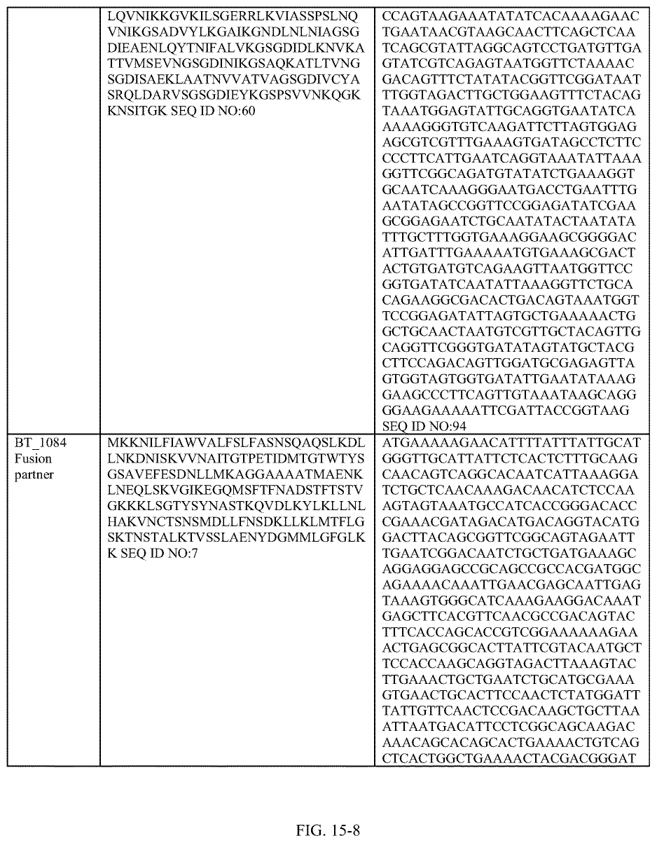

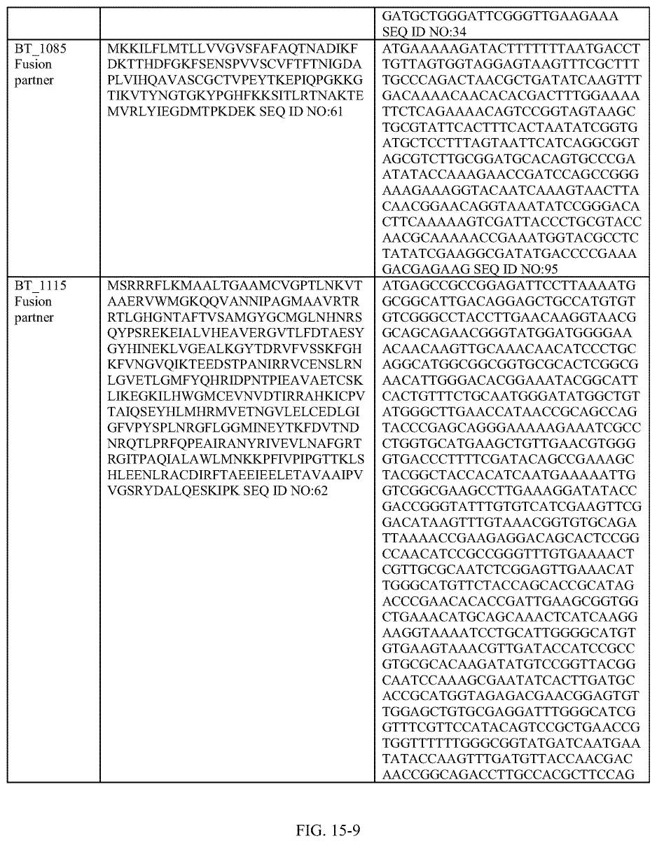

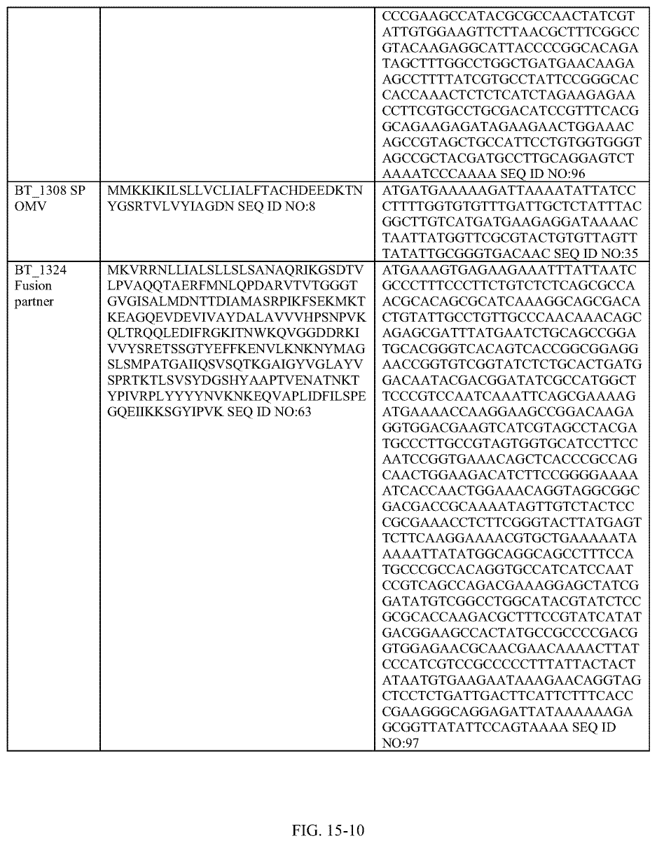

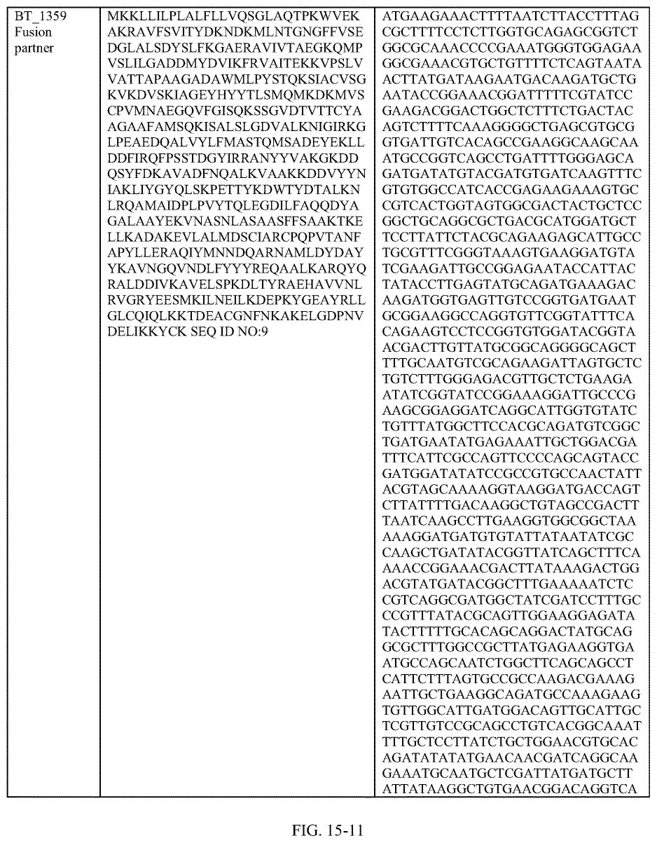

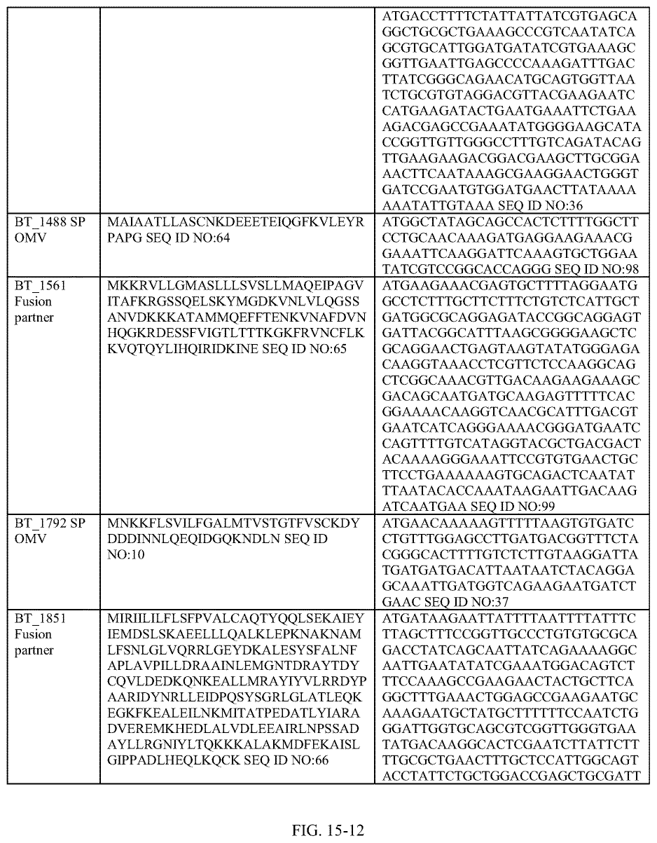

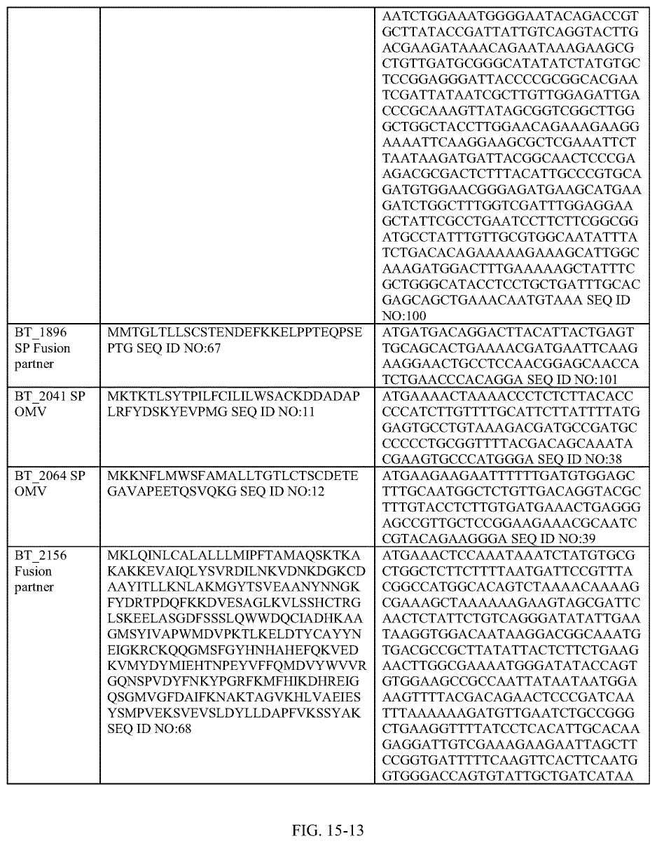

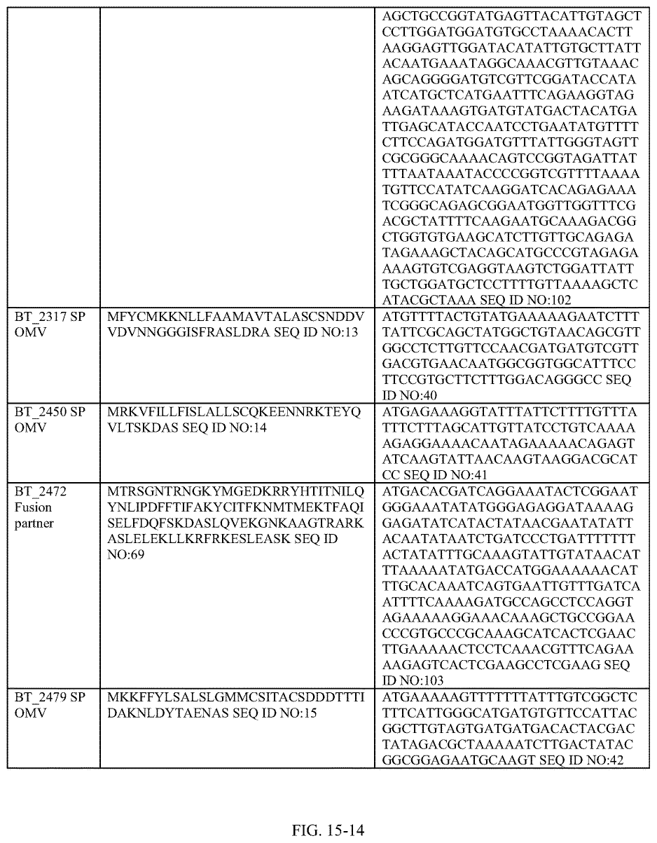

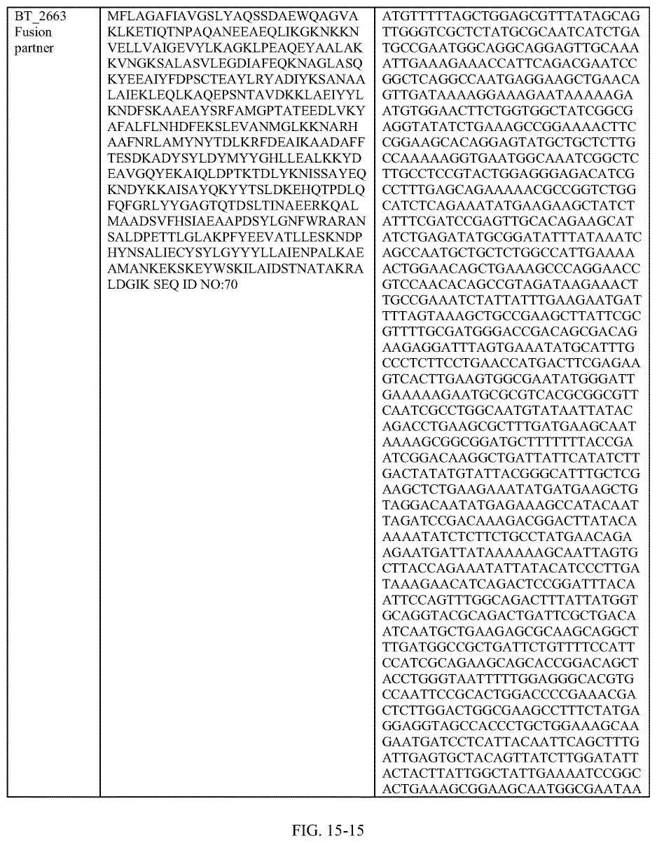

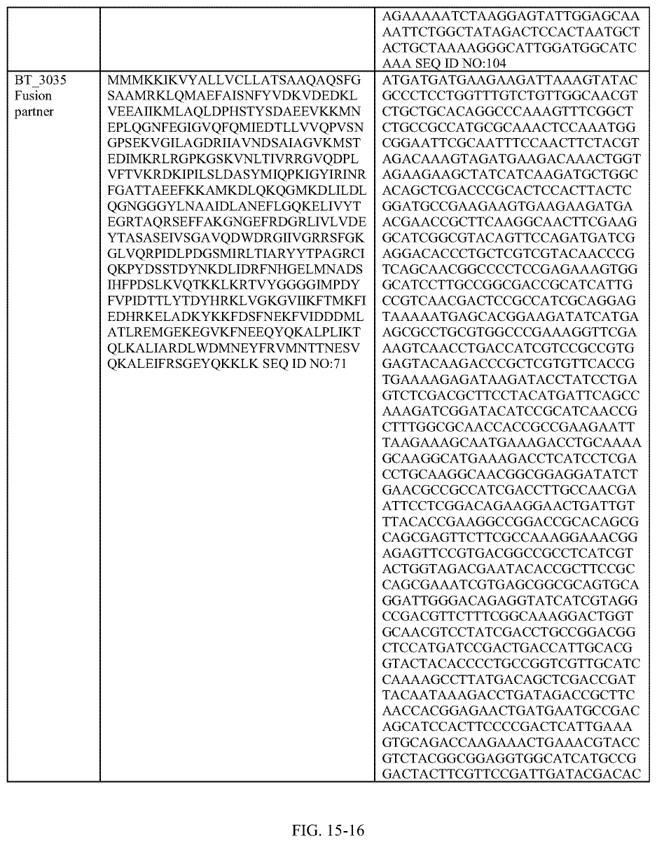

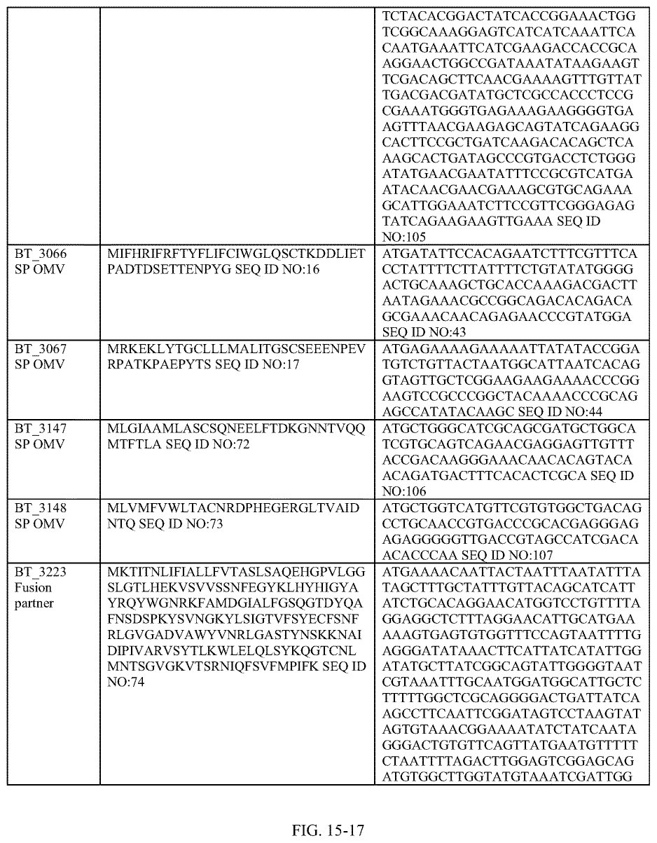

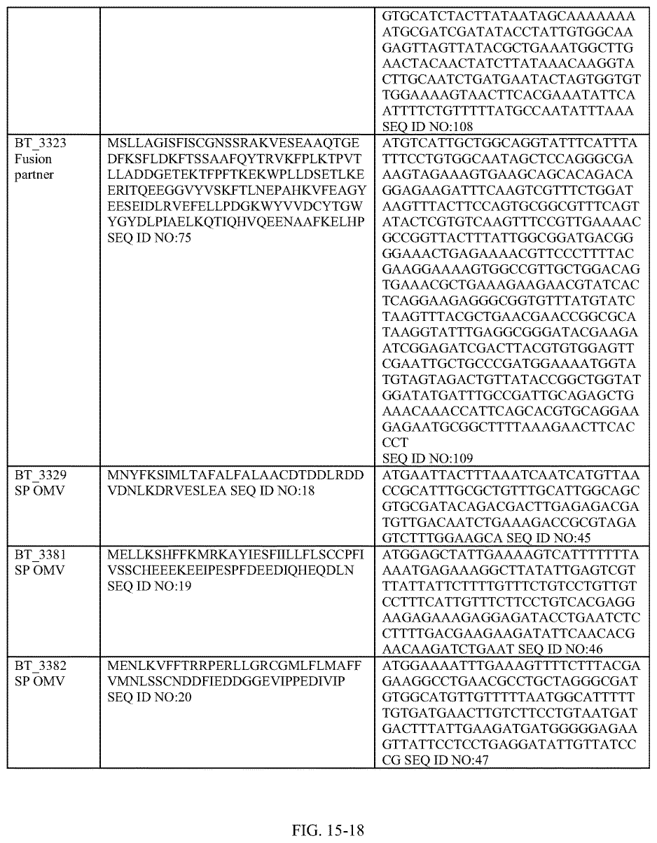

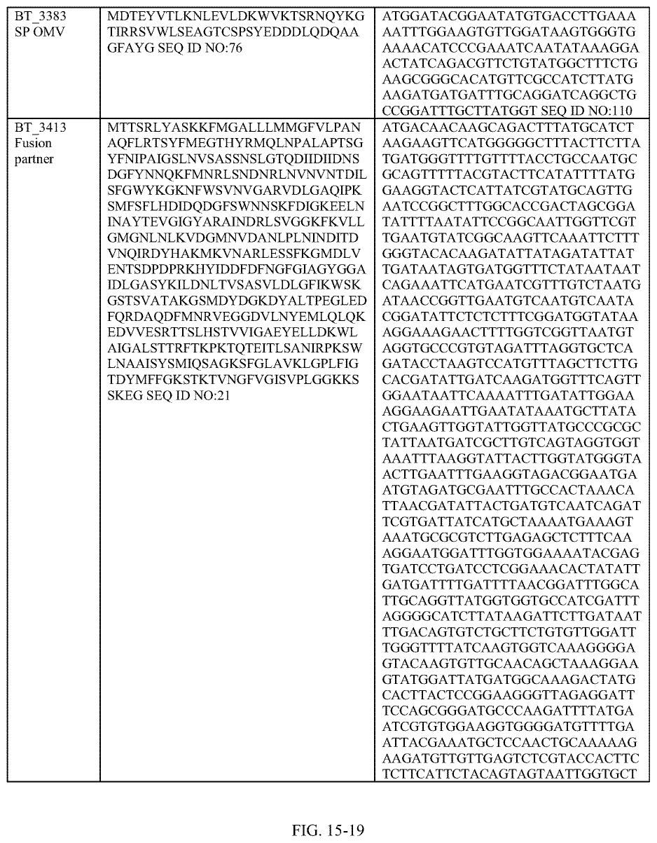

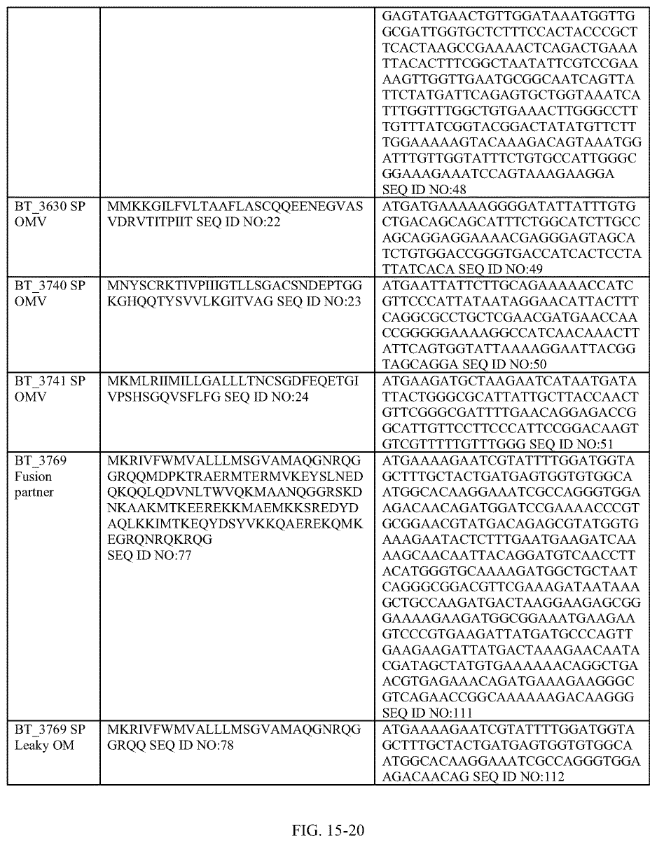

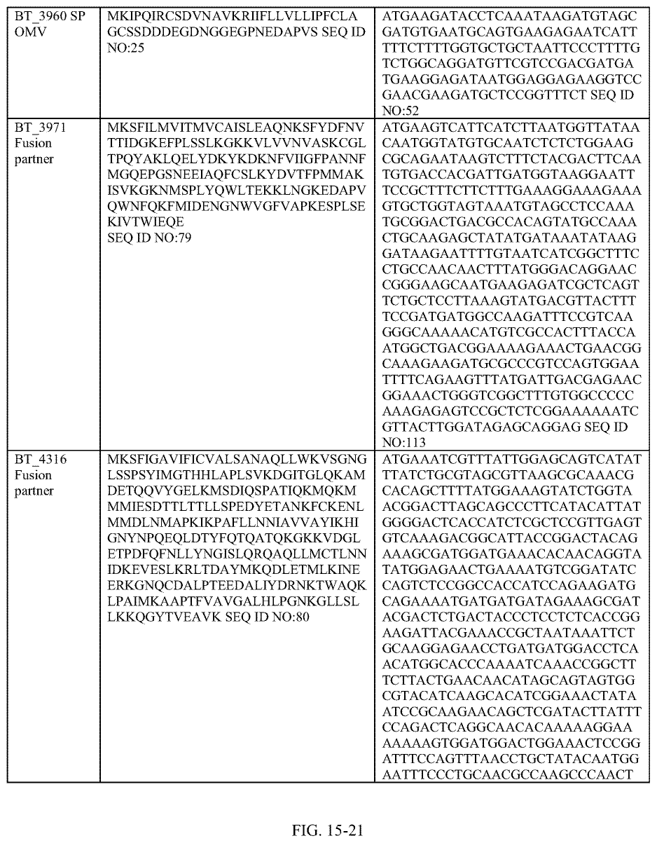

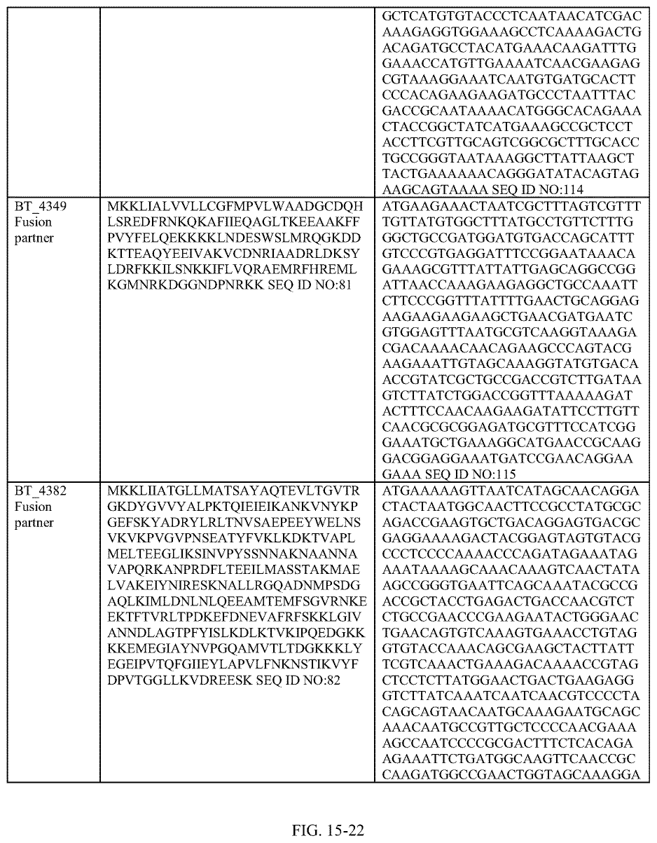

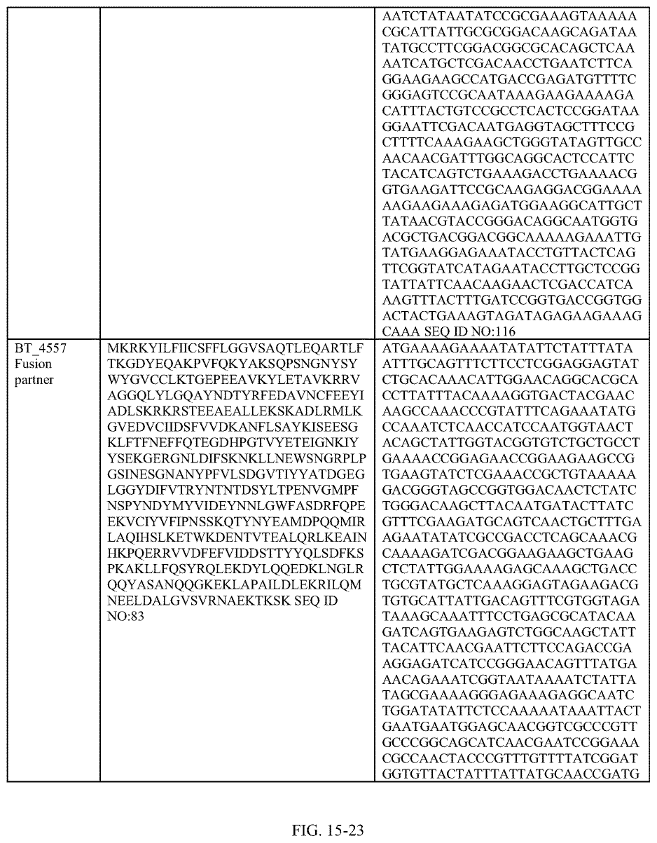

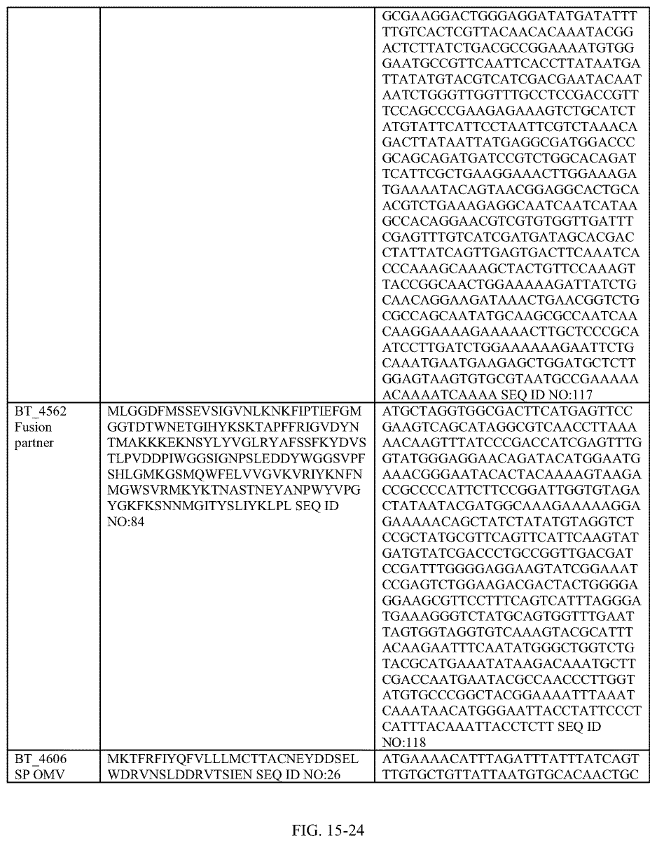

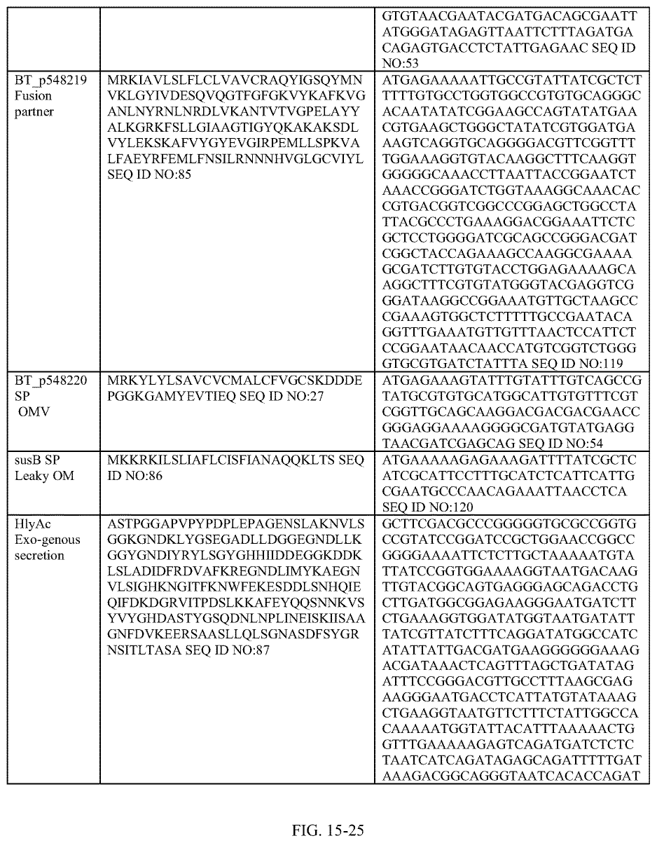

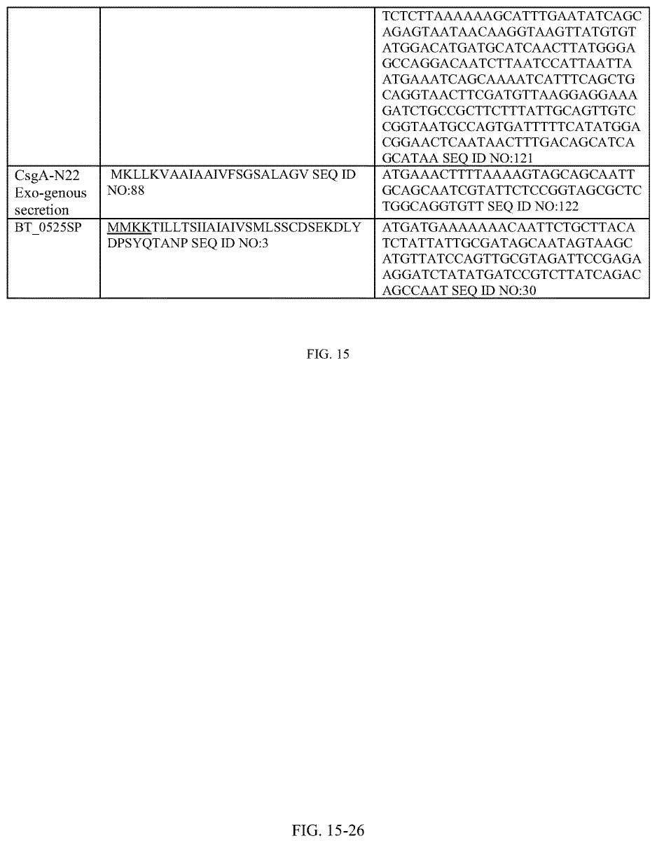

FIG. 15 shows sequences of constructs described herein.

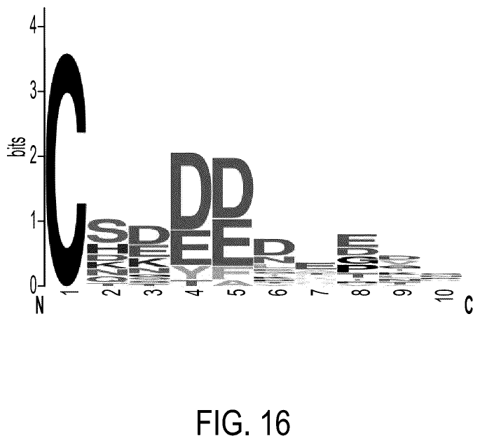

FIG. 16 shows both charged and uncharged polar residues are enriched in the LES of effective lipoprotein SPs. The first 10 amino acids after cleavage site of 19 effective lipoprotein SP are aligned using cysteine as the +1 position. The logo plot showing the conserved motifs was built by WebLogo (weblogo.berkeley.edu/logo.cgi)

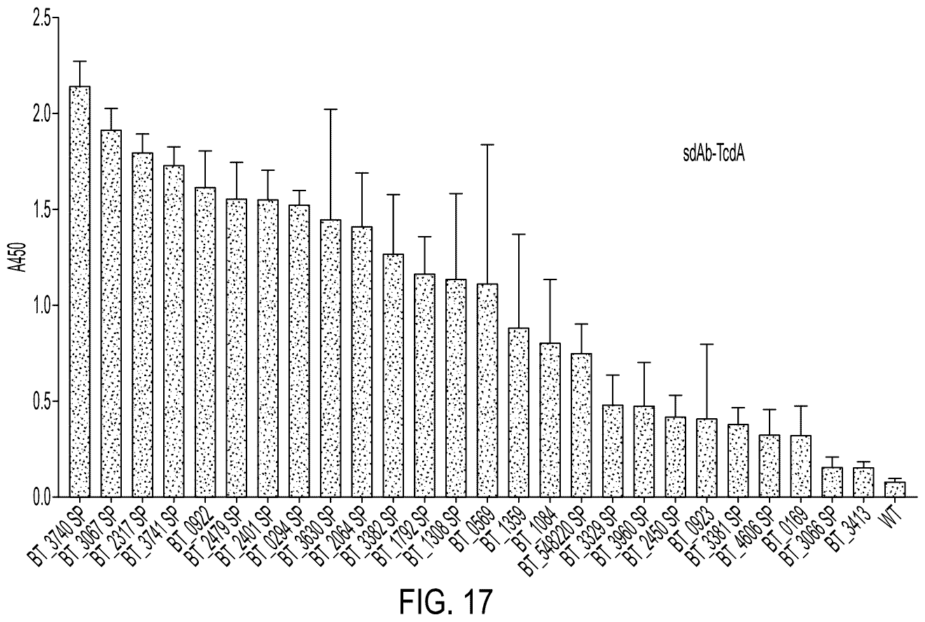

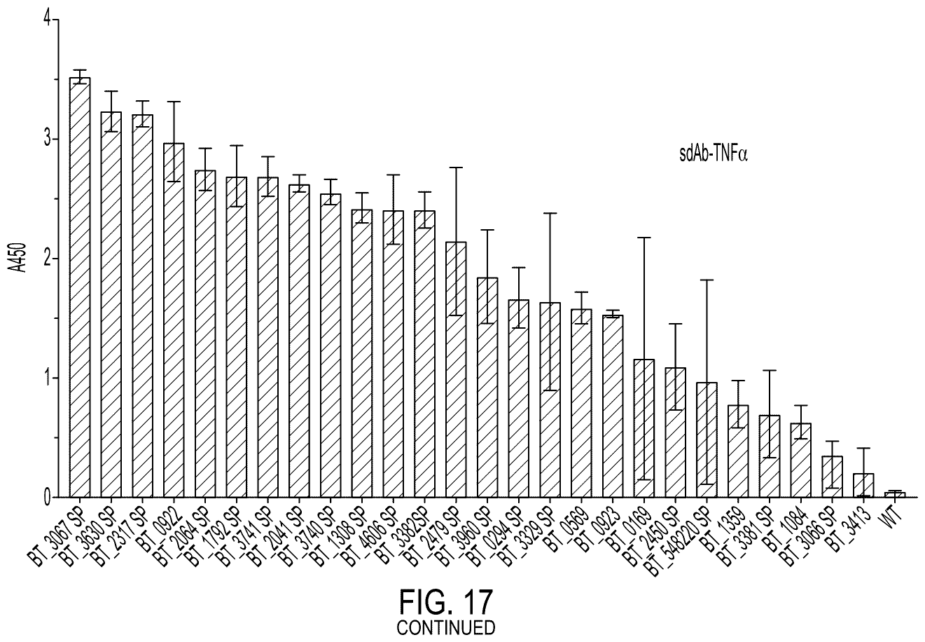

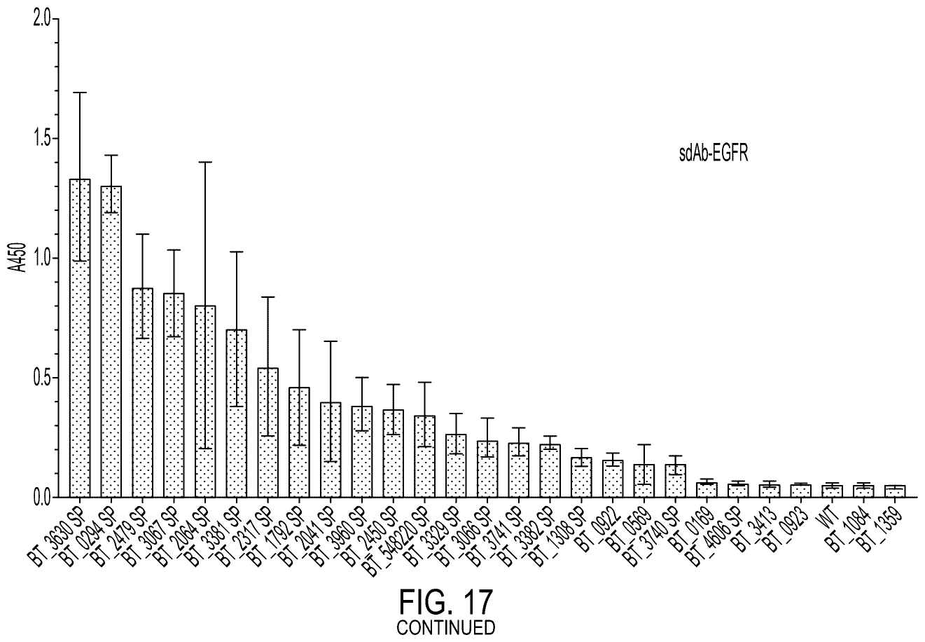

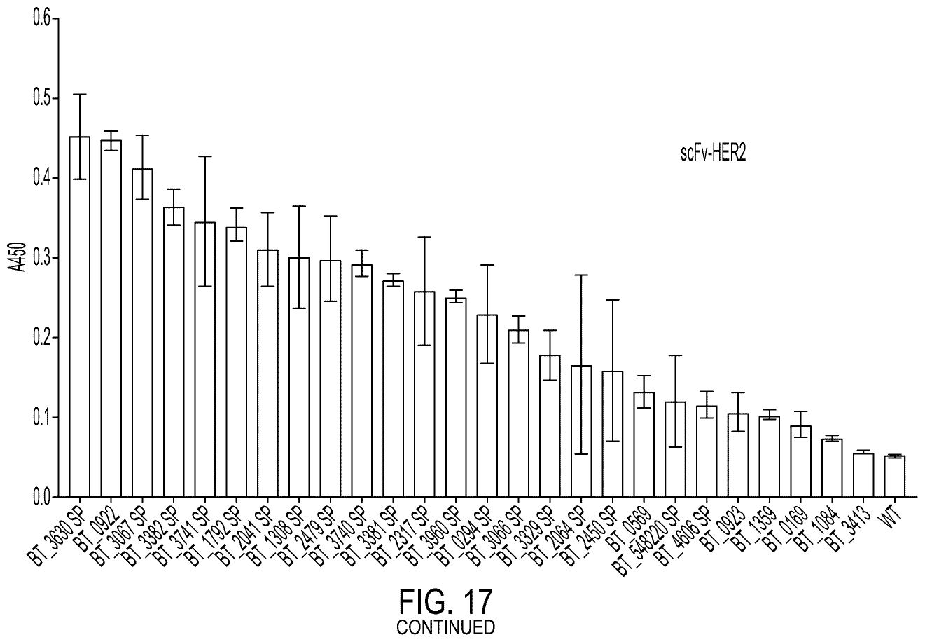

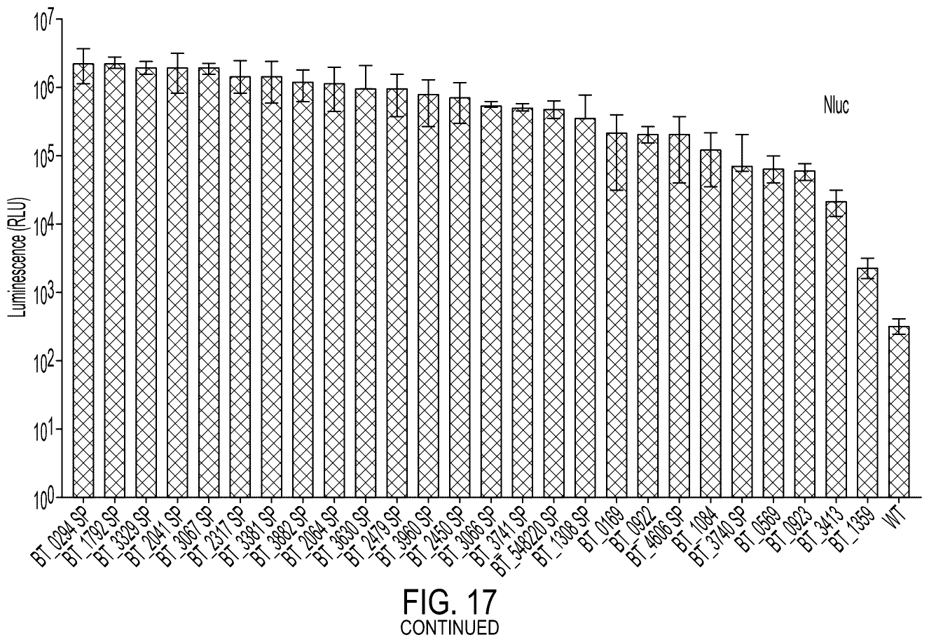

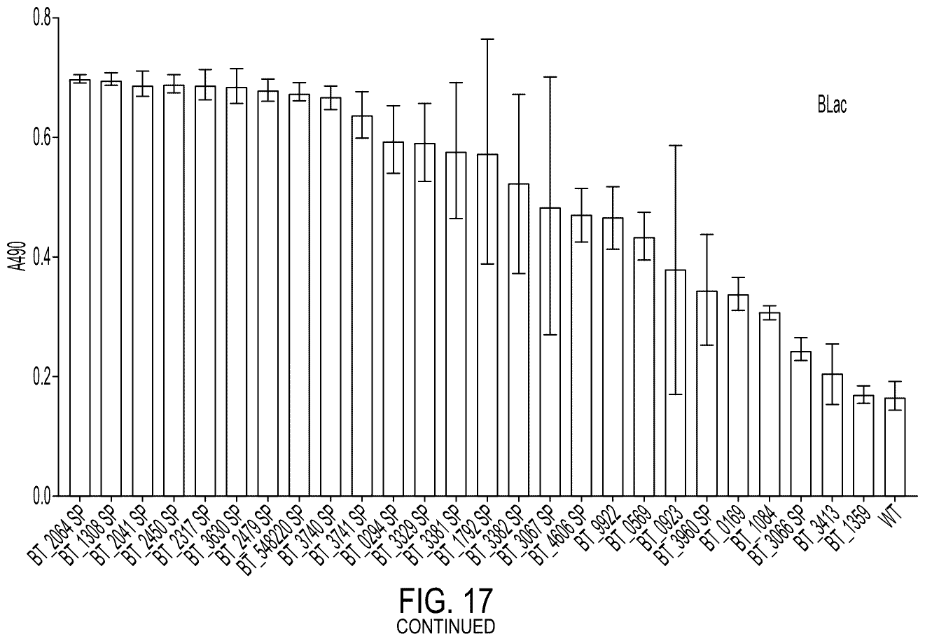

FIG. 17 shows activity assays of antibody fragments and reporter proteins secreted from B. theta. The activities of antibody fragments (sdAb-TcdA, sdAb-TNFα, sdAb-EGFR, and scFv-HER2) and reporter proteins (Nluc and BLac) secreted by 26 secretion carriers in B. theta culture supernatants were measured by their respective functional assays (ELISA for antibody fragments; luciferase assay for Nluc; colorimetric enzymatic assay for BLac). The secretion carriers were ranked based on their average readouts of functional assays.

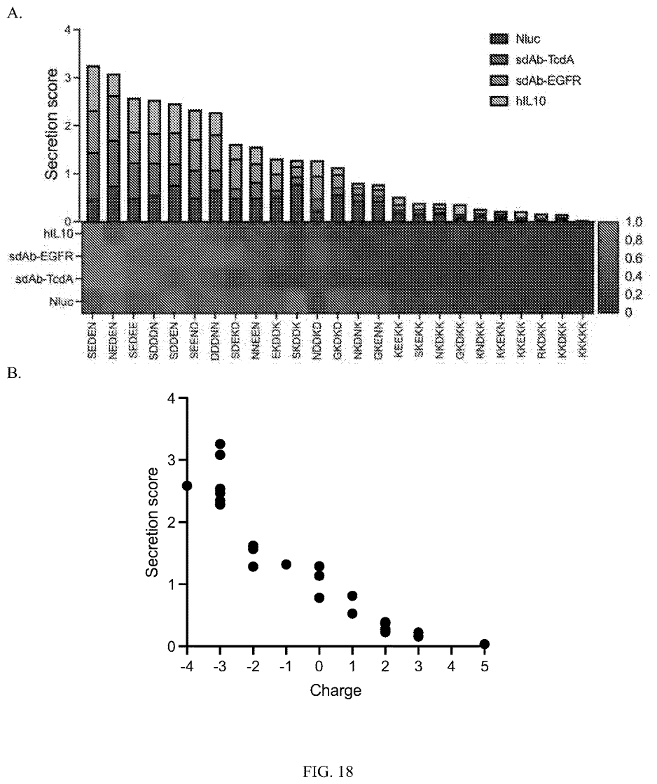

FIG. 18 shows BT_2479 SP-LES variants can fine-tune the secretion efficiency of various cargoes. (A) Direct ELISA of hIL10, sdAb-EGFR, sdAb-TcdA, and Nluc secreted into B. theta culture supernatant by different BT_2479 SP-LES variants. The readouts (A450) were converted to values between zero and one by cargo-wise min-max normalization. Secretion score was calculated by summing up the normalized readouts of four protein cargoes for each BT_2479 SP-LES variant. (B) Correlation between the secretion score of BT_2479 SP-LES variant and its LES net charge.

### DETAILED DESCRIPTION

Methods to enable heterologous protein secretion using both endogenous and exogenous secretion systems in Bacteroides, e.g., B. thetaiotaomicron (“B. theta”) are provided herein. Full-length proteins and lipoprotein signal peptides can be used as secretion carriers to export, e.g., functional antibody fragments, therapeutic proteins, and reporter proteins across multiple Bacteroides species at high titers. To provide a more complete understanding of these secretion tools, sequence features of lipoprotein signal peptides that were able to drive high levels of secretion of heterologous proteins, the post-secretion extracellular fate of different types of secretion carriers, and the cargo size limit of the lipoprotein signal peptides were characterized. To further increase the titers of secreted heterologous proteins and enable flexible control of the system, a strong, self-contained, inducible expression system was developed. Finally, the activity of the secretion carriers was characterized in vivo by observing the production and secretion of reporter proteins from engineered Bacteroides strains in the mouse gut. This toolkit expands the potential therapeutic impact of stably colonizing commensal bacterial strains, enabling them to deliver protein-based therapeutics from within the gut over long periods of time, which can support more effective treatment strategies for chronic gastrointestinal disease.

Provided herein are a suite of full-length proteins and lipoprotein SPs derived from native B. theta secretory proteins that can deliver functional antibody fragments, therapeutic proteins, and reporter proteins into the extracellular space. These secretion carriers are broadly functional across multiple Bacteroides species. Certain amino acid compositions of lipoprotein SPs can drive high-level secretion. The most effective SPs contain the following components: 1) a positively charged N-terminal region, 2) a central hydrophobic region with a minimum length requirement, and 3) a lipid export sequence (LES) that is enriched in both uncharged polar and negatively charged amino acids. The post-secretion fate of protein cargo exported via full-length fusion partners and lipoprotein SPs, occur by both OMV-dependent and OMV-independent secretion. By selecting specific secretion carriers secreted proteins can be directed to specific target destinations: freely soluble in the extracellular space; bound to the external surface of OMVs; or held within the OMV lumen. The molecular toolkit presented herein provides an accessible framework for generating living therapeutic and diagnostic machines from highly relevant human commensal Bacteroides species.

### Polypeptides

A polypeptide is a polymer where amide bonds covalently link three or more amino acids. A polypeptide can be post-translationally modified. A purified polypeptide is a polypeptide preparation that is substantially free of cellular material, other types of poly peptides, chemical precursors, chemicals used in synthesis of the polypeptide, or combinations thereof. A polypeptide preparation that is substantially free of cellular material, culture medium, chemical precursors, chemicals used in synthesis of the polypeptide has less than about 30%, 20%, 10%, 5%, 1% or less of other polypeptides, culture medium, chemical precursors, and/or other chemicals used in synthesis. Therefore, a purified polypeptide is about 70%, 80%, 90%, 95%, 99% or more pure.

The term “polypeptides” can refer to one or more types of polypeptides or a set of polypeptides. “Polypeptides” can also refer to mixtures of two or more different types of polypeptides including, but not limited to, full-length proteins, truncated polypeptides, or polypeptide fragments. The term “polypeptides” or “polypeptide” can each mean “one or more polypeptides.”

In one embodiment, a polypeptide or fragment thereof is non-naturally occurring. That is, a polypeptide or fragment thereof comprises 1, 2, 3, 4, 5, 10, 20, 30, 40, 50, 75 or more non-naturally occurring amino acids. In an embodiment, the non-naturally occurring amino acids can provide a beneficial property such as increased solubility of the polypeptide or increased sensitivity or increased specificity of the polypeptide in assays.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some embodiments the length of a reference sequence aligned for comparison purposes is at least 50, 60, 70, or 80% of the length of the comparison sequence, and in some embodiments is at least 90% or 100%. In an embodiment, the two sequences are the same length.

Ranges of desired degrees of sequence identity are approximately 80% to 100% and integer values in between. Percent identities between a disclosed sequence and a claimed sequence can be at least 80%, at least 83%, at least 85%, at least 90%, at least 95%, at least 98%, at least 99%, at least 99.5%, or at least 99.9%. In general, an exact match indicates 100% identity over the length of the reference sequence.

Polypeptides that are sufficiently similar to polypeptides described herein (e.g., SEQ ID NO:1-27, 123-125, 139-147, 150-179) can be used herein. Polypeptides that are about 90, 91, 92, 93, 94 95, 96, 97, 98, 99, 99.5% or more identical to polypeptides described herein can also be used herein.

A polypeptide variant differs by about, for example, 1, 2, 3, 4, 5, 6, 7, 8, 9, 10, 20, 30, 40, 50, 60, 70, 80, 90, or more amino acid residues (e.g., amino acid additions, substitutions, or deletions) from a peptide shown SEQ ID NOs:1-27, 123-125, 139-147, 150-179 or a fragment thereof. Where this comparison requires alignment, the sequences are aligned for maximum homology. The site of variation can occur anywhere in the polypeptide. In one embodiment, a variant has about 70%, 75%, 80%, 85%, 90%, 95%, 96%, 97%, 98%, 99% sequence identity to the original polypeptide.

In some aspects, a polypeptide comprises, consists essentially of, or consists of an amino acid sequence at least 65%, 70%, 75%, 80%, 85%, 90%, 95%, 96%, 97%, 98%, 99% or 100% (or any percentage in between) identical to the amino acid sequence put forth in SEQ ID NOs: 1-27, 123-125, 139-147, 150-179, or a fragment thereof. In some aspects, a polypeptide comprises, consists essentially of, or consists of an amino acid sequence at least 65%, 70%, 75%, 80%, 85%, 90%, 95%, 96%, 97%, 98%, 99% or 100% (or any percentage in between) identical to at least one portion of the amino acid sequence put forth in SEQ ID NOs:1-27, 123-125, 139-147, 150-179, or a fragment thereof.

Variant polypeptides can generally be identified by modifying one of the polypeptide sequences described herein and evaluating the properties of the modified polypeptide to determine if it is a biological equivalent. A variant is a biological equivalent if it reacts substantially the same as a polypeptide described herein in an assay such as an immunohistochemical assay, an enzyme-linked immunosorbent assay (ELISA), a turbidimetric immunoassay, a particle-enhanced turbidimetric immunoassay, a particle-enhanced turbidimetric immunoassay, a radioimmuno-assay (RIA), immunoenzyme assay, a western blot assay, or other suitable assay. Other suitable assays include those that test for the biological activity of the heterologous or therapeutic polypeptide or for the delivery of the heterologous polypeptide out of the cell. In other words, a variant is a biological equivalent if it has 90-110% of the activity of the original polypeptide.

Variant polypeptides can have one or more conservative amino acid variations or other minor modifications and retain biological activity, i.e., are biologically functional equivalents to SEQ ID NOs:1-27, 123-125, 139-147, 150-179, or a fragment thereof. Variant polypeptides can have labels, tags, additional Bacteroides amino acids, amino acids unrelated to Bacteroides, amino acids that can be used for purification, amino acids that can be used to increase solubility of the polypeptide, amino acids to improve other characteristics of the polypeptide, or other amino acids. In an embodiment, the additional amino acids are not Bacteroides amino acids.

Methods of introducing a mutation into an amino acid sequence are well known to those skilled in the art. See, e.g., Ausubel (ed.), Current Protocols in Molecular Biology, John Wiley and Sons, Inc. (1994); Maniatis et al., Molecular Cloning: A Laboratory Manual, Cold Spring Harbor laboratory, Cold Spring Harbor, N.Y. (1989). Mutations can also be introduced using commercially available kits such as “QuikChange™ Site-Directed Mutagenesis Kit” (Stratagene). The generation of a functionally active variant polypeptide by replacing an amino acid that does not influence the function of a polypeptide can be accomplished by one skilled in the art. A variant polypeptide can also be chemically synthesized.

Variant polypeptides can have conservative amino acid substitutions at one or more predicted nonessential amino acid residues. A conservative substitution is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. In one embodiment a polypeptide has about 1, 2, 3, 4, 5, 10, 20 or fewer conservative amino acid substitutions.

A polypeptide can be a fusion protein, which can contain other amino acid sequences, such as amino acid linkers, amino acid spacers, signal sequences, TMR stop transfer sequences, transmembrane domains, as well as ligands useful in protein purification, such as glutathione-S-transferase, histidine tag (e.g., about 6, 7, 8, 9, 10, or more His residues), and staphylococcal protein A, or combinations thereof. In an embodiment, a polypeptide comprises one or more epitope tags, such as FLAG (for example, DYKDDDDK; SEQ ID NO:126), HA (YPYDVPDYAC; SEQ ID NO:127), myc (EQKLISEEDLC; SEQ ID NO:128), V5 (GKPIPNPLLGLDST; SEQ ID NO:129), E-tag (GAPVPYPDPLEPR; SEQ ID NO:130), VSV-g (YTDIEMNRLGK; SEQ ID NO:131), 6×His (HHHHHHH; SEQ ID NO:132), and HSV (QPELAPEDPEDC; SEQ ID NO:133). An antibody, such as a monoclonal antibody, can specifically bind to an epitope tag and be used to purify a polypeptide comprising the epitope tag.

A fusion protein can comprise two or more different amino acid sequences operably linked to each other. A fusion protein construct can be synthesized chemically using organic compound synthesis techniques by joining individual polypeptide fragments together in fixed sequence. A fusion protein can also be chemically synthesized. A fusion protein construct can also be expressed by a genetically modified host cell (such as E. coli or Bacteroides) cultured in vitro, which carries an introduced expression vector bearing specified recombinant DNA sequences encoding the amino acids residues in proper sequence. The heterologous polypeptide, e.g., a therapeutic protein can be fused, for example, to the N-terminus or C-terminus of a secretion carrier polypeptide. More than one polypeptide can be present in a fusion protein. Fragments of polypeptides can be present in a fusion protein. A fusion protein can comprise, e.g., one, two, three, four, five, six, seven or more of an n-charged region, an LES region, a hydrophobic region, or a secretion carrier (e.g., SEQ ID NOs:1-27, 123-125, 139-147, 150-179, fragments thereof, or combinations thereof). A fusion protein can further comprise e.g., one, two, three, four, five, six, seven or more of a heterologous protein (e.g., a therapeutic or marker polypeptide). Polypeptides can be in a multimeric form. In other words, a polypeptide can comprise two or more copies (e.g., two, three, four, five, six, seven or more) of a secretion carrier, the components of a secretion carrier, a heterologous polypeptide, fragments thereof, or a combination thereof. A polypeptide can include, e.g., a fusion protein of two, three, four, five, six, seven or more polypeptides having about 70%, 75%, 80%, 85%, 90%, 95%, 96%, 97%, 98%, 99% or more sequence identity to SEQ ID NOs:1-27, 123-125, 139-147, 150-179; or a fusion protein of at least two polypeptides having about 70%, 75%, 80%, 85%, 90%, 95%, 96%, 97%, 98%, 99% sequence identity to SEQ ID NOs: 1-27, 123-125, 139-147, 150-179. A polypeptide can be a fusion protein that can include one or more linkers between the individual proteins making up the fusion protein. Alternatively, no linkers can be present between the individual proteins making up the fusion protein. A fusion polypeptide can contain other amino acid sequences, such as amino acid linkers, amino acid spacers, signal sequences, TMR stop transfer sequences, transmembrane domains, as well as ligands useful in protein purification, such as glutathione-S-transferase, histidine tag, epitope tags, and staphylococcal protein A, or combinations thereof.

Polypeptides can be lyophilized, desiccated, or dried, for example freeze-dried. A lyophilized polypeptide can be obtained by subjecting a preparation of the polypeptides to low temperatures to remove water from the sample. A desiccated polypeptide composition can be obtained by drying out a preparation of the polypeptides by removal of water. A dried polypeptide preparation can refer to a polypeptide preparation that has been air dried (e.g., lyophilized).

### Secretion Carrier

A secretion carrier can comprise a positively charged N-terminal region, a hydrophobic h-region, a cleavage site, and a lipoprotein export sequence. The secretion carrier can be operably linked or fused to a heterologous protein of interest (e.g., a therapeutic protein or a marker protein).

A positively charged N-terminal region can be about 3 to 9 amino acids (e.g., about 2, 3, 4, 5, 6, 7, 8, 9 10, or more amino acids). The positively charged N-terminal region can comprise a charge of greater than or equal to +1 (e.g., +1, +2, +3, +4, +5 or more). Positively charged amino acids include Lys, Arg, and His. Therefore, in an aspect a positively charged N-terminal region comprises 1, 2, 3, 4, 5, 6 or more amino acids selected from Lys, Arg, and His. In another aspect, a positively charged N-terminal region comprises: X₁X₂X₃X₄MKX₅X₆X₇, wherein X₁ is M or absent, X₂ is F or absent, X₃ is Y, M, or absent, X₄ is C, Y, or absent, X₅ is K, L, T, or I, X₆ is N, F, L, P, or K, X₇ is L, Q, or absent (SEQ ID NO:123). In some aspects if X₅ is I, then X₆ and X₇ are present. In an aspect, if X₅ is I, then X₆ is P, and X₇ is Q. In another aspect, a charged region comprises: MX₁X₂X₃X₄ wherein X₁ is R, N, E, I, or T, X₂ is K, N, Y, L, F, or T, X₃ is V, Y, L, E, S, F, L, H, or S, X₄ is K or R (SEQ ID NO:124). In an aspect, a positively charged N-terminal region can be any of those of BT_3741SP, BT_2064SP, BT2479SP, BT_0294SP, BT_3740SP, BT_3067SP, BT_2317SP, BT1359, BT_548220SP, BT_0922, BT_1792, BT_2450, BT_2041SP, BT_3960SP, BT_3382SP, BT_3381SP, BT_3329SP, BT_3630SP, BT_1084, BT_3066SP, BT_3413, BT_1308SP, BT_0569, BT_4606SP, BT_0923, or BT_0169, BT_0525SP. See FIG. 15.

A hydrophobic region can be about 13 to 34 amino acids in length (e.g., about 13, 14, 15, 16, 17, 18, 19, 20, 21, 22, 23, 24, 25, 26, 27, 28, 29, 30, 31, 32, 33, 34, 35, or more amino acids). Hydrophobic amino acids are: P, A, Y, G, I, M, W, V, F, and L. In an aspect, a hydrophobic region comprises 50, 60, 70, 80, 90, 95% or more hydrophobic amino acids. A hydrophobic region can be, for example, WLYACSLAIAFGVLSFVTVS (SEQ ID NO:139), TILLTSIIAIAIVSMLSS (SEQ ID NO:140), IYTLLIAFCAAWSLHS (SEQ ID NO:141), FLSVILFGALMTVSTGTFVS (SEQ ID NO:142), FFYLSALSLGMMCSITA (SEQ ID NO:143), LYTGCLLLMALITGS (SEQ ID NO:144), and MLRIIMILLGALLLTN (SEQ ID NO:145). In an aspect, a hydrophobic region can be any of those of BT_3741 SP, BT_2064SP, BT2479SP, BT_0294SP, BT_3740SP, BT_3067SP, BT_2317SP, BT1359, BT_548220SP, BT_0922, BT_1792, BT_2450, BT_2041SP, BT_3960SP, BT_3382SP, BT_3381SP, BT_3329SP, BT_3630SP, BT_1084, BT_3066SP, BT_3413, BT_1308SP, BT_0569, BT_4606SP, BT_0923, BT_0169, or BT_0525SP. See FIG. 15.

LES sequences can allow for secretion of the fusion protein from the host cell during expression. The polynucleotide sequence encoding the LES sequence can be operably linked to fusion protein DNA sequence, i.e., the two sequences are joined in the correct reading frame and positioned to direct the newly synthesized polypeptide into the secretory pathway of the host cell. Polynucleotide sequences encoding an LES can be positioned 5′ to the DNA sequence encoding a heterologous polypeptide of interest, although they can be positioned elsewhere in the DNA sequence of interest.

Uncharged polar residues in the LES can provide a secretion enhancing effect. The enrichment of uncharged polar residues (S/N/Q/T), specifically at positions +2 and +3 in the LES of effective lipoprotein SPs, may help promote more efficient packing of protein cargo into OMVs, resulting in the enhanced secretion levels. In an aspect, an LES has a S, N, Q, or T at positions +2 or +3.

In an aspect, a lipoprotein export sequence comprises a cleavage site. In an embodiment a lipoprotein export sequence comprises CX₁X₂X₃X₄X₅, wherein X₁ is S, K, N, R, S, E, D, or G, wherein X₂ is D, N, E, or K, wherein X₃ is D or E, wherein X₄ is D, N, E, or K, wherein X₅ is D, N, E, or K (SEQ ID NO:125). In an aspect, an LES comprises a majority of uncharged polar amino acids (e.g., serine (Ser), threonine (Thr), cysteine (Cys), asparagine (Asn), glutamine (Gin), and tyrosine (Tyr)) and negatively charged amino acids (e.g., aspartic acid (Asp) and glutamic acid (Glu)). In an aspect, an LES is about 4, 5, 6, 7, or more amino acids in length and comprises 2, 3, 4, 5, 6 or more amino acids selected from S, N, D, and E. In an aspect, an LES can be any of those of BT_3741 SP, BT_2064SP, BT2479SP, BT_0294SP, BT_3740SP, BT_3067SP, BT_2317SP, BT1359, BT_548220SP, BT_0922, BT_1792, BT_2450, BT_2041SP, BT_3960SP, BT_3382SP, BT_3381SP, BT_3329SP, BT_3630SP, BT_1084, BT_3066SP, BT_3413, BT_1308SP, BT_0569, BT_4606SP, BT_0923, BT_0169, or BT_0525SP. See FIG. 15.

In an aspect, an LES has a net charge of −4, −3, −2, or −1. In an embodiment the more negatively charged an LES region, the greater the secretion of a target polypeptide. See FIG. 18. By selecting an LES with more negative charge (e.g., −4 or −3), one can tune a system to secrete a greater amount of target polypeptide. If a lesser amount of secretion is desired a less negatively charged LES, a neutral charged LES, or even a positively charged LES can be selected (e.g. +1, +2, +3). See FIG. 18.

In an aspect, a secretion carrier comprises BT_3741 SP, BT_2064SP, BT2479SP, BT_0294SP, BT_3740SP, BT_3067SP, BT_2317SP, BT1359, BT_548220SP, BT_0922, BT_1792, BT_2450, BT_2041SP, BT_3960SP, BT_3382SP, BT_3381SP, BT_3329SP, BT_3630SP, BT_1084, BT_3066SP, BT_3413, BT_1308SP, BT_0569, BT_4606SP, BT_0923, BT_0169, or BT_0525SP. See FIG. 15

In an aspect a secretion carrier is a full-length membrane-associated Bacteroides lipoprotein or a truncated membrane-associated Bacteroides lipoprotein (e.g., SEQ ID NO:1-27).

### Promoters

A recombinant polynucleotide described herein can comprise a promoter. The term “promoter” and “promoter sequence” as used herein means a control sequence that is a region of a polynucleotide sequence at which the initiation and rate of transcription of a coding sequence, such as a gene or a transgene, are controlled. Promoters can be constitutive, inducible, repressible, or tissue-specific, for example. Promoters can contain genetic elements at which regulatory proteins and molecules such as RNA polymerase and transcription factors may bind. A promoter can be operably linked to a polynucleotide encoding a secretion carrier.

The term “operably linked” refers to the expression of a polynucleotide that is under the control of a promoter with which it is spatially connected. A promoter can be positioned 5′ (upstream) or 3′ (downstream) of a polynucleotide under its control. A promoter can be positioned 5′(upstream) of a gene under its control. The distance between a promoter and a polynucleotide can be approximately the same as the distance between that promoter and the polynucleotide it controls in the polynucleotide from which the promoter is derived. Variation in the distance between a promoter and a polynucleotide can be accommodated without loss of promoter function.

In some aspects, a promoter sequence can comprise, consist essentially of, or consist of a Bacteroides promoter sequence. A Bacteroides promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO:148.

A promoter can be a promoter derived from Bacteroides. A promoter can be an inducible promoter or a constitutive promoter. A promoter can be P_{BfP1E6}, P_{BfP3E1}, P_{BfP2E2}, P_{BfP2E3}, P_{BfP1 E4}, P_{BfP5E4}, P_{BfP2E5}, or P_{BfP4E5}. In an aspect, a promoter can be any promoter as described in US Pat. Publ. 20220160791, which is incorporated by reference herein.

The sequence of P_{BfP1E6} is:

SEQ ID NO: 148
CAATTGGGCTACCTTTTTTTTGTTTTGTTTGCAATGGTTAATCTA
TTGTTAAAATTTAAAGTTTCACTTGAACTTTCAAATAATGTTCTT
ATATTTGCAGTGTCGAAAGAAACAAAGTAG.

### Ribosome Binding Site (RBS)

A ribosome binding site (RBS) is a sequence within mRNA that is bound by the ribosome when initiating protein translation. An RBS can be located at about between −5 and −11 or at about −8 from a start codon. Most RBS sequences have at least four bases of an AGGAGG core motif.

A polynucleotide described herein can comprise a nucleotide sequence encoding a ribosome binding site (RBS). A sequence encoding an RBS can be operably linked to a promoter and can be positioned between the promoter and the nucleotide sequence encoding a secretion carrier and a therapeutic polypeptide. In some aspects, an RBS is positioned 3′ of a promoter. In some aspects, an RBS is positioned 5′ of the nucleotide sequence encoding a secretion carrier and a therapeutic protein. In some cases, an RBS is positioned 3′ of the promoter and 5′ of the nucleotide sequence encoding a secretion carrier and a therapeutic protein.

Additionally, RBS having about 70, 80, 85, 90, 95, 96, 97, 98, 99% or more sequence identity to the nucleotide sequences set forth in SEQ ID NO:149 can be used. A RBS can be any suitable RBS. An RBS can be any RBS as described in US Pat. Publ. 20220160791 (e.g., RBS1, RBS2, RBS3, RBS4, RBS5, RBS6, RBS7, or RBS8). In an aspect an RBS IS RBS1 (GACTGATCGGCGCGACTCACGCGCCGATCAGTAATG; SEQ ID NO:202), RBS2 (GACTGATCAGGAAGAGTAAAAAATATTAAAATAATG SEQ ID NO:203); RBS3 (GACTGATCTCTGGGGTGAATAAAATTTATAATAATG SEQ ID NO:204); RBS4 (GACTGATCCCCCATTCTATTAAATTTTAGAATAATG SEQ ID NO:205); RBS5 (GACTGATCGGTGTTAGCTTTAAATATTAGAATAATG SEQ ID NO:206); RBS6 (GACTGATCTAGCACTCTTAAAAAAATTAAAATAATG SEQ ID NO:207); RBS7 (GACTGATCGTAATCTTTAAAAAAAATAAAAATAATG); or RBS8: GACTGATCGTCCATCAATTTAAAATTTAAAATAATG SEQ ID NO:149. Other suitable RBSs are disclosed in Mimee et al., Programming a Human Commensal Bacterium, Bacteroides thetaiotaomicron, to Sense and Respond to Stimuli in the Murine Gut Microbiota. Cell Syst. 2015 Jul. 29; 1(1):62-71. One of ordinary skill in the art can select a suitable RBS using the techniques described herein.

### Therapeutic Polypeptides

A polynucleotide encoding a secretion carrier can be fused or operably linked to a polynucleotide encoding any polypeptide, including, for example, marker proteins or therapeutic proteins such as antibodies or specific binding fragments thereof, cytokines, or growth factors. An antibody or specific binding fragment thereof can be a scFv, Fab, Fab′, Fv, F(ab′)₂, a minibody, a diabody, a triabody, a tetrabody, a tandem di-scFv, a tandem tri-scFv, an immunoglobulin single variable domain (ISV), such as, a V_{HH} (including humanized V_{HH}), a camelized V_{H}, a single domain antibody, a domain antibody, or a dAb.

A therapeutic antibody polypeptide can include a V_{L} domain and a V_{H} domain, a V_{H} domain or suitable light, heavy, or light and heavy CDRs from, for example, 3F8, Abagovomab, Abciximab, Abituzumab, Abrezekimab, Abrilumab, Actoxumab, Adalimumab, Adecatumumab, Aducanumab, Afasevikumab, Afelimomab, Alacizumab pegol, Alemtuzumab, Alirocumab, Altumomab pentetate, Amatuximab, Amivantamab, Anatumomab mafenatox, Andecaliximab, Anetumab ravtansine, Anifrolumab, Ansuvimab, Anrukinzumab, Apolizumab, Aprutumab ixadotin, Arcitumomab, Ascrinvacumab, Aselizumab, Atezolizumab, Atidortoxumab, Atinumab, Atoltivimab, Atoltivimab/maftivimab/odesivimab, Atorolimumab, Avelumab, Azintuxizumab vedotin, Bamlanivimab, Bapineuzumab, Basiliximab, Bavituximab, Bebtelovimab, Bectumomab, Begelomab, Belantamab mafodotin, Belimumab, Bemarituzumab, Benralizumab, Berlimatoxumab, Bermekimab, Bersanlimab, Bertilimumab, Besilesomab, Bevacizumab, Bezlotoxumab, Biciromab, Bimagrumab, Bimekizumab, Birtamimab, Bivatuzumab, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Camidanlumab tesirine, Camrelizumab, Canakinumab, Cantuzumab mertansine, Cantuzumab ravtansine, Caplacizumab, Casirivimab, Capromab, Carlumab, Carotuximab, Catumaxomab, Cedelizumab, Cemiplimab, Cergutuzumab amunaleukin, Certolizumab pegol, Cetrelimab, Cetuximab, Cibisatamab, Cilgavimab, Cirmtuzumab, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, Crizanlizumab, Crotedumab, Cusatuzumab, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dezamizumab, Dinutuximab, Dinutuximab beta, Diridavumab, Domagrozumab, Dorlimomab aritox, Dostarlimab, Drozitumab, Duligotuzumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emapalumab, Emibetuzumab, Emicizumab, Enapotamab vedotin, Enavatuzumab, Enfortumab vedotin, Enlimomab pegol, Enoblituzumab, Enokizumab, Enoticumab, Ensituximab, Epcoritamab, Epitumomab cituxetan, Epratuzumab, Eptinezumab, Erenumab, Erlizumab, Ertumaxomab, Etaracizumab, Etesevimab, Etigilimab, Etrolizumab, Evinacumab, Evolocumab, Exbivirumab, Fanolesomab, Faralimomab, Faricimab, Farletuzumab, Fasinumab, Felvizumab, Fezakinumab, Fibatuzumab, Ficlatuzumab, Figitumumab, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, Gancotamab, Ganitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, lanalumab, Ibalizumab, Sintilimab, Ibritumomab tiuxetan, Icrucumab, Idarucizumab, Ifabotuzumab, Igovomab, Iladatuzumab vedotin, Imalumab, Imaprelimab, Imciromab, Imdevimab, Imgatuzumab, Inclacumab, Indatuximab ravtansine, Indusatumab vedotin, Inebilizumab, Infliximab, Intetumumab, Inolimomab, Inotuzumab ozogamicin, Ipilimumab, lomab-B, Iratumumab, Isatuximab, Iscalimab, Istiratumab, Itolizumab, Ixekizumab, Keliximab, Labetuzumab, Lacnotuzumab, Ladiratuzumab vedotin, Lampalizumab, Lanadelumab, Landogrozumab, Laprituximab emtansine, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Loncastuximab tesirine, Losatuxizumab vedotin, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lupartumab, Lupartumab amadotin, Lutikizumab, Maftivimab, Mapatumumab, Margetuximab, Marstacimab, Maslimomab, Mavrilimumab, Matuzumab, Mepolizumab, Metelimumab, Milatuzumab, Minretumomab, Mirikizumab, Mirvetuximab soravtansine, Mitumomab, Modotuximab, Mogamulizumab, Monalizumab, Morolimumab, Mosunetuzumab, Motavizumab, Moxetumomab pasudotox, Muromonab-CD, Nacolomab tafenatox, Namilumab, Naptumomab estafenatox, Naratuximab emtansine, Narnatumab, Natalizumab, Navicixizumab, Navivumab, Naxitamab, Nebacumab, Necitumumab, Nemolizumab, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odesivimab, Odulimomab, Ofatumumab, Olaratumab, Oleclumab, Olendalizumab, Olokizumab, Omalizumab, Omburtamab, Onartuzumab, Ontuxizumab, Onvatilimab, Opicinumab, Oportuzumab monatox, Oregovomab, Orticumab, Otelixizumab, Otilimab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab vedotin, Pintumomab, Placulumab, Prezalumab, Plozalizumab, Pogalizumab, Polatuzumab vedotin, Ponezumab, Porgaviximab, Prasinezumab, Prezalizumab, Priliximab, Pritoxaximab, Pritumumab, Quilizumab, Racotumomab, Radretumab, Rafivirumab, Ralpancizumab, Ramucirumab, Ranevetmab, Ranibizumab, Raxibacumab, Ravagalimab, Ravulizumab, Refanezumab, Regavirumab, Regdanvimab, Relatlimab, Remtolumab, Reslizumab, Rilotumumab, Rinucumab, Risankizumab, Rituximab, Rivabazumab pegol, Robatumumab, Rmab, Roledumab, Romilkimab, Romosozumab, Rontalizumab, Rosmantuzumab, Rovalpituzumab tesirine, Rovelizumab, Rozanolixizumab, Ruplizumab, Sacituzumab govitecan, Samalizumab, Samrotamab vedotin, Sarilumab, Satralizumab, Satumomab pendetide, Secukinumab, Selicrelumab, Seribantumab, Setoxaximab, Setrusumab, Sevirumab, Sibrotuzumab, Sifalimumab, Siltuximab, Simtuzumab, Siplizumab, Sirtratumab vedotin, Sirukumab, Sofituzumab vedotin, Solanezumab, Solitomab, Sonepcizumab, Sontuzumab, Sotrovimab, Spartalizumab, Stamulumab, Sulesomab, Suptavumab, Sutimlimab, Suvizumab, Suvratoxumab, Tabalumab, Tacatuzumab tetraxetan, Tadocizumab, Tafasitamab, Talacotuzumab, Talizumab, Talquetamab, Tamtuvetmab, Tanezumab, Taplitumomab paptox, Tarextumab, Tavolimab, Teclistamab, Tefibazumab, Telimomab aritox, Telisotuzumab, Telisotuzumab vedotin, Tenatumomab, Teneliximab, Teplizumab, Tepoditamab, Teprotumumab, Tesidolumab, Tetulomab, Tezepelumab, Tibulizumab, Tildrakizumab, Tigatuzumab, Timigutuzumab, Timolumab, Tiragolumab, Tiragotumab, Tislelizumab, Tisotumab vedotin, Tixagevimab, Tocilizumab, Tomuzotuximab, Toralizumab, Tosatoxumab, Tositumomab, Tovetumab, Tralokinumab, Trastuzumab, Trastuzumab duocarmazine, Trastuzumab emtansine, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Vunakizumab, Xentuzumab, Zalutumumab, Zanolimumab, Zatuximab, Zenocutuzumab, Ziralimumab, Zolbetuximab, or Zolimomab aritox.

Beneficial cytokines include, for example, Acrp30, AgRP, amphiregulin, angiopoietin-1, AXL, BDNF, bFGF, BLC, BMP-4, BMP-6, b-NGF, BTC, CCL28, Ck beta 8-1, CNTF, CTACK CTAC, Skinkine, Dtk, ENA-78, eotaxin, eotaxin-2, MPIF-2, eotaxin-3, MIP-4-alpha, Fas, Fas/TNFRSF6/Apo-1/CD95, FGF-4, FGF-6, FGF-7, FGF-9, Flt-3 Ligand fms-like tyrosine kinase-3, FKN or FK, GCP-2, GCSF, GDNF Glial, GITR, GITR, GM-CSF, GRO, GRO-α, HCC-4, hematopoietic growth factor, hepatocyte growth factor, 1-309, ICAM-1, ICAM-3, IFN-γ, IGFBP-1, IGFBP-2, IGFBP-3, IGFBP-4, IGFBP-6, IGF-I, IGF-I SR, IL-1α, IL-1β, IL-1, IL-1 R4, ST2, IL-3, IL-4, IL-5, IL-6, IL-8, IL-10, IL-11, IL-12 p40, IL-12p70, IL-13, IL-16, IL-17, I-TAC, alpha chemoattractant, lymphotactin, MCP-1, MCP-2, MCP-3, MCP-4, M-CSF, MDC, MIF, MIG, MIP-1α, MIP-1β, MIP-1δ, MIP-3α, MIP-3β, MSP-a, NAP-2, NT-3, NT-4, osteoprotegerin, oncostatin M, PARC, P1GF, RANTES, SCF, SDF-1, soluble glycoprotein 130, soluble TNF receptor I, soluble TNF receptor II, TARC, TECK, TIMP-1, TIMP-2, TNF-α, TNF-β, thrombopoietin, TRAIL R3, TRAIL R4, and uPAR.

Beneficial growth factors include, for example, transforming growth factors, e.g., transforming growth factor beta 1 and 2 (TGF-β1,2) and TGF-α, Epidermal growth factor (EGF), or keratinocyte growth factor (KGF). vascular endothelial growth factor (VEGF), and platelet-derived growth factor (PDGF).

### Cleavage Sites and Proteases

A polynucleotide encoding a polypeptide such as a therapeutic protein or a marker protein can be fused or operably linked to a secretion carrier, which includes a linker or cleavage site. In some aspects, a polynucleotide encodes a linker or cleavage site positioned between the secretion carrier and the heterologous polypeptide (e.g., a therapeutic or marker polypeptide). In some aspects, a cleavage site can be present within the secretion carrier (e.g., between the hydrophobic region and the LES region). In some aspects, a linker can be a cleavable linker. In some cases, a cleavable linker can be a self-cleaving linker (e.g., a 2A peptide or an intein). In some aspects a cleavable linker or cleavage site can be cleavable by one or more proteases present within the gastrointestinal tract of a subject. Where a therapeutic polypeptide linked to a secretion carrier comprises a cleavable site or cleavable linker that is cleavable by one or more proteases present within the gastrointestinal tract of a subject, the therapeutic polypeptide will be released from the secretion carrier after secretion and when the extracellular environment includes a corresponding protease.

In some aspects, a cleavable linker is cleavable by one or more host cell proteases (e.g., proteases of a Bacteroides cell or proteases of a cell of the host animal's gut) (e.g., an extracellular protease such as a matrix metalloproteinase, or an endopeptidase-2; an intracellular protease such as a cysteine protease or a seine protease; etc.). For example, a polypeptide can be fused to a secretion carrier as disclosed herein such that the fusion protein is incorporated into outer membrane vesicles (OMVs) that are released from the Bacteroides cell and then fuse with a subject's cell, thus delivering the polypeptide of interest into the cytoplasm of a subject's cell. In this case a cleavable linker can be cleavable by a eukaryotic cytoplasmic protease. In another aspect, where a secretion carrier comprises a polypeptide (e.g., a therapeutic polypeptide) fused to a secretion carrier via a linker that is cleavable by one or more host cell proteases (e.g., an extracellular and/or intracellular host cell protease), the polypeptide will be released from the secretion carrier after secretion and when the environment (e.g., subject's cell's cytoplasm) includes an appropriate corresponding protease. In another aspect, a polypeptide can be fused to a secretion carrier such that the fusion protein is excreted from a recombinant Bacteroides cell in a subject's gut, thus delivering the polypeptide into the subject's gut. In this case a cleavable linker can be cleavable by a protease present in the subject's gut.

Any convenient cleavable linker can be used. In an aspect a cleavable linker or cleavage site can be cleaved by a gut or eukaryotic protease such as chymotrypsin-like elastase family member 2A, anionic trypsin-2, chymotrypsin-C, chymotrypsinogen B, elastase 1, elastase 3, trypsin, and chymotrypsin (e.g., chymotrypsin B). Thus, in some cases, a cleavable linker of a secreted fusion protein is cleavable by one or more gut proteases such as a trypsin, a chymotrypsin, and an elastase. In some cases, a cleavable linker of a subject secreted fusion protein is cleavable by one or more gut proteases selected from: chymotrypsin-like elastase family member 2A (cleavage site: Leu (L), Met (M) and Phe (F)), anionic trypsin-2 (cleavage site: Arg (R), Lys (K)), chymotrypsin-C (cleavage site: Leu (L), Tyr (Y), Phe (F), Met (M) Trp (W), Gln (Q), Asn (N)), chymotrypsinogen B (cleavage site: Tyr (Y), Trp (W), Phe (F), Leu (L)), elastase 1 (cleavage site: Ala (A)), and elastase 3 (cleavage site: Ala (A)).

A cleavable linker or cleavage site can have any suitable length. In some cases, a cleavable linker or cleavage site is about 1, 2, 5, 10, 15, or more amino acids in length.

Cleavage sites for gut proteases include, for example: Chymotrypsin A; followed by A; followed by a P or a V; followed by an FYL, or W. Examples of suitable cleavage sites include, trypsin: SGPTGHGR (SEQ ID NO:134), trypsin: SGPTGMAR (SEQ ID NO:135), chymotrypsin: SGPTASPL (SEQ ID NO:136), chymotrypsin B: SGPTTAPF (SEQ ID NO:137), elastase I: SGPTAAPA (SEQ ID NO:138).

### Recombinant Polynucleotides

Recombinant polynucleotides contain less than an entire microbial genome and can be single- or double-stranded nucleic acids. A polynucleotide can be RNA, DNA, cDNA, genomic DNA, chemically synthesized RNA or DNA or combinations thereof. A polynucleotide can comprise, for example, a gene, open reading frame, non-coding region, or regulatory element.

A gene is any polynucleotide molecule that encodes a polypeptide, protein, or fragments thereof, optionally including one or more regulatory elements preceding (5′ non-coding sequences) and following (3′ non-coding sequences) the coding sequence. In one embodiment, a gene does not include regulatory elements preceding and following the coding sequence. A native or wild-type gene refers to a gene as found in nature, optionally with its own regulatory elements preceding and following the coding sequence. A chimeric or recombinant gene refers to any gene that is not a native or wild-type gene, optionally comprising regulatory elements preceding and following the coding sequence, wherein the coding sequences and/or the regulatory elements, in whole or in part, are not found together in nature. Thus, a chimeric gene or recombinant gene comprise regulatory elements and coding sequences that are derived from different sources, or regulatory elements and coding sequences that are derived from the same source but arranged differently than is found in nature. A gene can encompass full-length gene sequences (e.g., as found in nature and/or a gene sequence encoding a full-length polypeptide or protein) and can also encompass partial gene sequences (e.g., a fragment of the gene sequence found in nature and/or a gene sequence encoding a protein or fragment of a polypeptide or protein). A gene can include modified gene sequences (e.g., modified as compared to the sequence found in nature). Thus, a gene is not limited to the natural or full-length gene sequence found in nature.

Polynucleotides can be purified free of other components, such as proteins, lipids and other polynucleotides. For example, the polynucleotide can be 50%, 75%, 90%, 95%, 96%, 97%, 98%, 99%, or 100% purified. A polynucleotide existing among hundreds to millions of other polynucleotide molecules within, for example, cDNA or genomic libraries, or gel slices containing a genomic DNA restriction digest are not to be considered a purified polynucleotide. Polynucleotides can encode the polypeptides described herein (e.g., SEQ ID NO:1-27, 123-125, 139-147, 150-179).

Polynucleotides can comprise additional heterologous nucleotides that do not naturally occur contiguously with the polynucleotides. As used herein the term “heterologous” refers to a combination of elements that are not naturally occurring or that are obtained from different sources.

Polynucleotides can be isolated. An isolated polynucleotide is a naturally-occurring polynucleotide that is not immediately contiguous with one or both of the 5′ and 3′ flanking genomic sequences that it is naturally associated with. An isolated polynucleotide can be, for example, a recombinant DNA molecule of any length, provided that the nucleic acid sequences naturally found immediately flanking the recombinant DNA molecule in a naturally-occurring genome is removed or absent. Isolated polynucleotides also include non-naturally occurring nucleic acid molecules. Polynucleotides can encode full-length polypeptides, polypeptide fragments, and variant or fusion polypeptides.

Degenerate polynucleotide sequences encoding polypeptides described herein, as well as homologous nucleotide sequences that are at least about 80, or about 85, 90, 91, 92, 93, 94, 95, 96, 97, 98, or 99% identical to polynucleotides described herein and the complements thereof are also polynucleotides. Degenerate nucleotide sequences are polynucleotides that encode a polypeptide described herein or fragments thereof, but differ in nucleic acid sequence from the wild-type polynucleotide sequence, due to the degeneracy of the genetic code. Complementary DNA (cDNA) molecules, species homologs, and variants of polynucleotides that encode biologically functional polypeptides also are polynucleotides.

Polynucleotides can be obtained from nucleic acid sequences present in, for example, a yeast or bacteria. Polynucleotides can also be synthesized in the laboratory, for example, using an automatic synthesizer. An amplification method such as PCR can be used to amplify polynucleotides from either genomic DNA or cDNA encoding the polypeptides.

Polynucleotides can comprise non-coding sequences or coding sequences for naturally occurring polypeptides or can encode altered sequences that do not occur in nature.

Unless otherwise indicated, the term polynucleotide or gene includes reference to the specified sequence as well as the complementary sequence thereof.

The expression products of genes or polynucleotides are often proteins, or polypeptides, but in non-protein coding genes such as rRNA genes or tRNA genes, the product is a functional RNA. The process of gene expression is used by all known life forms, i.e., eukaryotes (including multicellular organisms), prokaryotes (bacteria and archaea), and viruses, to generate the macromolecular machinery for life. Several steps in the gene expression process can be modulated, including the transcription, up-regulation, RNA splicing, translation, and post-translational modification of a protein.

A polynucleotide can be a cDNA sequence or a genomic sequence. A “genomic sequence” is a sequence that is present or that can be found in the genome of an organism or a sequence that has been isolated from the genome of an organism. A cDNA polynucleotide can include one or more of the introns of a genomic sequence from which the cDNA sequence is derived. As another example, a cDNA sequence can include all of the introns of the genomic sequence from which the cDNA sequence is derived. Complete or partial intron sequences can be included in a cDNA sequence.

Polynucleotides as set forth in SEQ ID NO:28 through SEQ ID NO:54, a functional fragment thereof; or having at least 95% identity to SEQ ID NO:28 through SEQ ID NO:54, are provided herein. In some embodiments, the isolated polynucleotides have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, and any number or range in between, identity to SEQ ID NO:28 through SEQ ID NO:54 or a functional fragment thereof. A polynucleotide can comprise a promoter, RBS, and encode a secretion carrier, n-charged region, hydrophobic h-region, LES and a heterologous polypeptide.

### Vectors

A vector is a polynucleotide that can be used to introduce polynucleotides or expression cassettes into one or more host cells. Vectors include cloning vectors, expression vectors, shuttle vectors, plasmids, cassettes, and the like. Any suitable vector can be used to deliver polynucleotides or expression cassettes to a population of host cells.

A plasmid is a circular double-stranded DNA construct used as a cloning and/or expression vector. Some plasmids can take the form of an extrachromosomal self-replicating genetic element (episomal plasmid) when introduced into a host cell. Other plasmids integrate into a host cell chromosome when introduced into a host cell. Expression vectors can direct the expression of polynucleotides to which they are operatively linked. Expression vectors can cause host cells to express polynucleotides and/or polypeptides other than those native to the host cells, or in a non-naturally occurring manner in the host cells. Some vectors may result in the integration of one or more polynucleotides (e.g., recombinant polynucleotides) into the genome of a host cell.

Polynucleotides or expression cassettes (e.g., one or more of a promoter, RBS, and polynucleotides encoding a secretion carrier, n-charged region, hydrophobic h-region, LES and/or a heterologous polypeptide) can be cloned into an expression vector optionally comprising expression control elements, including for example, origins of replication, promoters, enhancers, or other regulatory elements that drive expression of the polynucleotides or expression cassettes in host cells. One or more polynucleotides or expression cassettes can be present in the same vector. Alternatively, each polynucleotide or expression cassette can be present in a different vector.

### Cells

Polynucleotides encoding secretion carriers fused or operably linked to a heterologous polypeptide, such as a therapeutic or marker polypeptide can be delivered to a host by any suitable method to generate a recombinant cell that can secrete the heterologous polypeptide. A cell can be, for example, a Bacteroides cell such as a B. thetaiotaomicron, B. ovatus, B. fragilis, B. vulgatus, B. distasonis, or B. uniformis cell. Other Bacteroides cells can be used such as B. acidifaciens, B. barnesiaes, B. caccae, B. caecicola, B. caecigallinarum, B. cellulosilyticus, B. cellulosolvens, B. clarus, B. coagulans, B. coprocola, B. coprophilus, B. coprosuis, B. dorei, B. eggerthii, B. gracilis, B. faecichinchillae, B. faecis, B. finegoldii, B. fluxus, B. galacturonicus, B. gallinaceum, B. gallinarum, B. goldsteinii, B. graminisolvens, B. helcogene, B. intestinalis, B. luti, B. massiliensis, B. nordii, B. oleiciplenus, B. oris, B. paurosaccharolyticus, B. plebeius, B. polypragmatus, B. propionicifaciens, B. putredinis, B. pyogenes, B. reticulotermitis, B. rodentium, B. salanitronis, B. salyersiae, B. sartorii, B. sedimenti, B. stercoris, B. suis, B. tectus, or B. xylanisolvens.

In an aspect, a cell can secrete about 0.01, 0.1, 1.0, 10, 50, 100 μg/mL or more. of a heterologous polypeptide, such as a therapeutic or marker polypeptide into cell culture media. In an aspect, a cell can secrete about 0.01, 0.1, 1.0, 5, 10 mg/mL or more. of a heterologous polypeptide, such as a therapeutic or marker polypeptide into cell culture media.

In an aspect, a heterologous protein can have a molecular weight of about 10, 20, 30, 40, 50, 60, 65, 68, 70, 75, 85, 88, 90 kDa or more.

### Methods of Treatment

Methods of treatment include administering a recombinant cell comprising a secretion carrier operably linked or fused to a polypeptide, such as a therapeutic polypeptide to a subject (e.g., a human, a non-human animal, or a mammal). The subject can have an intestinal disorder such as inflammatory bowel disease (IBD) or Crohn's disease. The cell can be administered orally, intrarectally, or by any other suitable method.

In some aspects, a recombinant cell as described herein can be used to deliver a protein to another cell, e.g., a eukaryotic cell. In some aspects, a recombinant cell as described herein can be used to deliver a heterologous protein to another cell in vitro or in vivo. In some aspects, a heterologous protein can be delivered to an immune cell in vitro or in vivo. For example, a heterologous protein can be delivered to a B cell, a dendritic cell, a granulocyte, a megakaryocyte, a monocytes/macrophage, a natural killer cell, a platelet, a red blood cell, a T cell or a thymocyte. In some aspects, an immune cell is an intestinal mucosal immune cell. An intestinal mucosal immune cell is a component of the mucosal immune system at the gastrointestinal barrier, which contains small foci of lymphocytes and plasma cells that are scattered widely throughout the lamina propria of the gut wall.

To deliver a recombinant protein to another cell, a Bacteroides OMV can interact with the cell, e.g., the immune cell. Recombinant proteins can be delivered to a cell by being displayed on the surface of a Bacteroides OMV, which is recognized by a receptor on the surface of a cell, e.g., an immune cell, receiving the recombinant protein. In some aspects, the Bacteroides OMV undergoes lysis and releases the recombinant protein to the vicinity of the cell receiving the fusion protein. In some aspects, an Bacteroides OMV undergoes membrane fusion with the cell receiving the fusion protein. In some aspects, a Bacteroides OMV is internalized as a whole entity by the cell receiving the fusion protein via endocytosis. Polynucleotides, polypeptides, vectors, and cells described herein can be for use in a method of treating the human or animal body by therapy. For example, intestinal disorders such as inflammatory bowel disease (IBD) or Crohn's disease can be treated.

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### EXAMPLES

### Example 1 Materials and Methods

### Bacterial Strains and Culture

Bacteroides thetaiotaomicron VPI-5482, Bacteroides fragilis NCTC 9343, Bacteroides ovatus ATCC 8483, and Bacteroides vulgatus ATCC 8482 were acquired from ATCC. Bacteroides species were anaerobically cultured at 37° C. in TYG medium, BHIS medium (Brain Heart Infusion Supplemented with 1 μg/ml menadione, 0.5 mg/ml cysteine, 0.2 mM histidine, 1.9 mM hematin) or on BHI agar with 10% horse blood (BHIB). E. coli strains were aerobically cultured in LB medium at 37° C. E. coli DH5a was used for plasmid maintenance and E. coli RK231 (107) was used to achieve plasmid transfer in Bacteroides strains via tri-parental mating. Antibiotics were used when required at the following concentrations: ampicillin 100 μg/mL, kanamycin 50 μg/mL, gentamicin 20-200 μg/mL, and erythromycin 10-25 μg/mL.

### Molecular Cloning

Q5 high-fidelity DNA polymerase (New England Biolabs) was used for PCR amplification of DNA fragments for cloning. All primers were synthesized by Integrated DNA Technologies (IDT). All plasmid construction was done by Gibson Assembly (HiFi DNA Assembly Master Mix, New England Biolabs) and validated by colony PCR and sequencing. Plasmids were stored in E. coli DH5a for maintenance and conjugation. All endogenous secretion carriers were cloned from the genome of B. theta. The hlyA, hlyB, hlyD of UPEC T1SS were cloned from pVDL9.3 (Addgene #168299) and pEHlyA5 (Addgene #168298) plasmids. The csgG of E. coli K-12 T8SS was cloned from the genome of E. coli DH5a. The sequences of the N-terminal 22 residues of CsgA, SusB signal peptide, and BT_3769 signal peptide were introduced at the N-terminus of sdAb-TcdA directly through primers. The toxin A fragment (TcdAf; amino acid residues 2460-2710) was amplified from the C. difficle genome by PCR and cloned into the 2Bc-T plasmid (Addgene #37236). The sequences of PBfP1E6, sdAb-TcdA, VHH3, and EGFP were synthesized by IDT. The sequences of Nluc and 7D12 were cloned from plasmids pNBU2_erm-TetR-P1T_DP-GH023-NanoLuc (Addgene #117728) and pTrcHIS-wt7D12 (Addgene #125268). The anti-HER2 scFv was constructed from trastuzumab as previously described (71).

### Conjugation and Selection

To introduce plasmids into Bacteroides strains, they were first used to transform E. coli DH55α to generate plasmid donor E. coli strains. Overnight cultures of E. coli DH5a (plasmid donor), E. coli RK231 (helper strain), and Bacteroides (plasmid recipient) were combined in a 1:1:1 ratio of volume. The mixed liquid cultures were pelleted by centrifugation at 10,000×g for 1 min, resuspended in 30 μL LB medium, spotted onto on BHIB plates, and incubated aerobically for 24 hr at 37° C. The mating spots were scraped off the plates and streaked onto BHIB plates supplemented with selective antibiotics (200 μg/mL gentamicin and 25 μg/mL erythromycin), and incubated anaerobically for 2-3 days at 37° C. to allow selective growth of transconjugant Bacteroides clones.

### Recombinant Protein Expression and Purification

The HER2 extracellular domain was purified as previously described (71). For sdAb-TcdA and toxin A fragment purification, an overnight culture of E. coli BL21(DE3) harboring pET24b(+)-sdAb-TcdA-3×FLAG-6×His or 2Bc-T-TcdAf plasmids was grown overnight at 37° C. with shaking, then diluted 50-fold in 50 mL terrific broth with 50 μg/ml kanamycin. When culture OD₆₀₀ reached 0.6, IPTG was added to a final concentration of 0.1 mM to induce protein expression. After overnight induction of cultures at 25° C. with shaking, the cells were harvested and sonicated in lysis buffer (20 mM sodium phosphate, 0.5 M NaCl, 40 mM imidazole, 1% Triton X100, 0.1 mM PMSF pH 7.4). The soluble fractions of cell lysates were passed through a Ni-NTA chromatography column, and the sdAb-TcdA-3×FLAG-6×His recombinant proteins were eluted with elution buffer (20 mM sodium phosphate, 0.5 M NaCl, and 500 mM imidazole). The concentration of purified proteins was calculated from A280.

### Quantification of Protein Secretion Levels by Dot Blot Analysis

Bacteroides strains were first streaked on BHIB plate with antibiotics (200 μg/mL gentamicin and 25 μg/mL erythromycin). Colonies were inoculated into TYG or BHIS media with 12.5 μg/mL erythromycin (100 ng/mL aTc was additionally supplemented when using aTc-inducible promoters). Bacteroides strains harboring plasmids with constitutive promoters were grown to stationary phase while those with aTc-inducible promoters were grown to early log phase. The culture supernatants were separated from bacterial cells by centrifugation at 10,000×g for one minute and filtered through 0.22 μm syringe filters. For dot blot analysis, 10-30 μL of supernatant was directly spotted onto a PVDF membrane. For western blot analysis, 10 μL of supernatant was subjected to SDS-PAGE for protein separation then transferred to a PVDF membrane. The membrane was then blocked with 5% milk in PBST (phosphate-buffered saline with 0.1% Tween® 20 (polysorbate)) at room temperature for 1 hr, then incubated with anti-FLAG M2 monoclonal antibody (Sigma-Aldrich, 1:2000 dilution in 5% milk) at 4° C. overnight. After washing three times with PBS-T, the membrane was incubated with goat anti-mouse IgG secondary antibody conjugated with horse radish peroxidase (HRP) (Jackson Immuno Research, 1:5000 dilution in 5% milk) at room temperature for 1 hr. Signal was detected using SuperSigna™ West Dura Extended Duration Substrate (Thermo Scientific #34075) on a GelDoc imaging system.

### Measurement of Activities of Secreted Antibody Fragments and Reporters in Culture Supernatants

“Activity” is defined as antigen binding for antibody fragments and enzymatic or fluorescent activity for reporter enzymes. The activities of all antibody fragments were measured by ELISA as follows: 96-well immunoplates were coated with purified antigens (2 μg/ml) at 4° C. overnight. After washing with 0.1% PBS-T, microplates were blocked with 5% milk/0.1% PBS-T for 1 hr at room temperature (RT). Filtered culture supernatants were added to wells and incubated for 1 hr at RT. The microplates were washed again, and anti-FLAG M2 antibody (Sigma-Aldrich, 1:2000 dilution in 5% milk/0.1% PBS-T) was added and incubated for 1 hr at RT. Following PBS-T washing, goat anti-mouse IgG secondary antibody conjugated with HRP (Jackson Immuno Research, 1:5000 dilution in 5% milk/0.1% PBS-T) were added and incubated for 1 hr at RT. After washing with PBS-T, o-phenylenediamine (OPD) substrate solution was added and allowed to react for 30 min at RT. The absorbance at 450 nm (A450) was measured using a BioTek Synergy HT multimode microplate reader. A standard curve of sdAb-TcdA was generated using purified recombinant sdAb-TcdA-3×FLAG-6×His. For NanoLuc, the Nano-Glo luciferase assay (Promega) was performed as follows: 10 μL filtered culture supernatant was mixed with 15 μL PBS, followed by mixing with 25 μL NanoLuc reaction buffer supplemented with substrates at a ratio of 1:50. Luminescence was measured on the microplate reader using an integration time of 1 s and gain of 100. For β-lactamase, the β-lactamase activity assay kit (Sigma-Aldrich) was used following the manufacturer's protocol. Briefly, 10 μL filtered culture supernatant was mixed with 40 μL PBS, followed by mixing with 50 μL P-Lactamase assay buffer supplemented with substrates at a ratio of 1:25. After incubation for 5 min, the absorbance at 490 nm (A490) was measured using the microplate reader.

### Isolations of Supernatant, Pellet, and OMV Fractions

B. theta colonies were inoculated into TYG with 12.5 μg/mL erythromycin and anaerobically grown to late log phase or stationary phase. Cell pellets from 1 mL of liquid culture were collected by centrifugation at 10000×g for 1 min to obtain “total supernatant” fractions. Pellets were washed once with PBS to obtain “cell pellet” fractions. The “total supernatant” fractions were further centrifuged at 7000×g for 5 min then filtered through 0.22 μm syringe filters to obtain cell-free supernatants. OMVs were extracted using the ExoBacteria™ OMV Isolation Kit (System Biosciences) according to the manufacturer's protocol. Flow-through fractions from the OMV-binding columns were then centrifuged at 100,000×g for 3 hr to remove the remaining unbound OMV, and the supernatants were collected as OMV-free supernatants.

### Animal Experiments

All animal experiments were performed using protocols approved by the University of Illinois Institutional Animal Care and Use Committee. C57BL/6 specific-pathogen-free (SPF) mice (6-8 weeks old; sex-balanced) were pre-treated with an antibiotic cocktail in their drinking water (1 g/L metronidazole, 1 g/L neomycin, 0.5 g/L vancomycin, and 1 g/L ampicillin, and 20 g/L Kool-Aid Drink Mix) for 7 days followed by a 2-day washout period with plain tap water. Mice were then divided into four groups (2 males and 2 females per group) and administered 200 μL of the following treatments by oral gavage (1) PBS, (2) B. theta WT, (3) B. theta constitutively expressing Nluc, or (4) B. theta constitutively expressing BT_0294 SP-Nluc. All bacterial samples contained 1*10⁹ CFU bacterial cells. Mice were weighed and the fecal samples were collected for 60 days. To quantify colonization of the engineered B. theta strains, fecal samples was homogenized in PBS, serially diluted in 96-well plates, and plated on selective BHIB plates to calculate CFU. Because our WT strain did not contain a plasmid with a selectable marker, we could not quantify colonization levels of that strain using this approach. For measuring the presence of secreted Nluc in the fecal pellets, the homogenized fecal solutions were centrifuged at 12000×g for 2 min to pellet bacterial cells, then 25 μL of supernatant was used in the Nano-Glo luciferase assay described above.

### Statistical Analysis

All experiments were performed in duplicate or triplicate. Significance was tested by unpaired two-tailed Welch's t test in Excel. The values are presented as the mean of replicates ±standard deviation. *p<0.05, **p<0.01, ***p<0.001

### Example 2 P_{BfP1E6}-RBS8 Promoter/RBS Drives Strong and Reproducible Protein Secretion in B. theta

To establish a set of secretion tools for members of the Bacteroides genus, we selected Bacteroides thetaiotaomicron (B. theta) as the starting point based on its prevalence and abundance in the human gut. To ensure robust and reproducible results, we first sought to establish a framework for evaluation of protein expression and secretion across diverse samples. We identified a core set of three native B. theta proteins that are highly secreted, each with a different N-terminal signal sequence: BT_2472 (Sec SP), BT_3382 (lipoprotein SP), and BT_3769 (no SP identified; secretion mechanism unknown). To identify optimal genetic parts for reproducible and detectable protein secretion, we tested each protein using three different promoter/ribosome-binding site (RBS) pairs (FIG. 1A). For strong, constitutive expression, we used a Bacteroides fragilis (B. fragilis) phage promoter paired with either its original ribosome-binding site (RBS_{phage}) or a modified version with the highest observed activity amongst reported variants (RBS8). For more precisely controlled protein expression, we used a tightly regulated anhydrotetracycline (aTc)-inducible promoter (P1T_{DP}-GH023).

The P1T_{DP} promoter sequence is shown below:

SEQ ID NO: 196
TTTGCACCCGCTTTCCAAGAGAAGAAAGCCTTGTTAAATTGACTT
AGTGTAAAAGCGCAGTACTGCTTGACCATAAGAACAAAAAAATCT
CTATCACTGATAGGGATAAAGTTTGGAAGATAAAGCTAAAAGTTC
TTATCTTTGCAGTCTCCCTATCAGTGATAGAGA.

The P1T_{DP}-GH023 (promoter+RBS) sequence (without tetR expression cassette) is shown below:

SEQ ID NO: 197
TTTTGCACCCGCTTTCCAAGAGAAGAAAGCCTTGTTAAATTGACT
TAGTGTAAAAGCGCAGTACTGCTTGACCATAAGAACAAAAAAATC
TCTATCACTGATAGGGATAAAGTTTGGAAGATAAAGCTAAAAGTT
CTTATCTTTGCAGTCTCCCTATCAGTGATAGAGACGAAATAAAGA
CATATAAAAGAAAAGACAC.

The sequence of P2-A21-tetR-P1TDP-GH023 sequence (with tetR expression cassette) is shown below:

SEQ ID NO: 198
CTTAACGGCTGACATGGGAATTCCCCTCCACCGCGGTGGCTTAAG
ACCCACTTTCACATTTAAGTTGTTTTTCTAATCCGCATATGATCA
ATTCAAGGCCGAATAAGAAGGCTGGCTCTGCACCTTGGTGATCAA
ATAATTCGATAGCTTGTCGTAATAATGGCGGCATACTATCAGTAG
TAGGTGTTTCCCTTTCTTCTTTAGCGACTTGATGCTCTTGATCTT
CCAATACGCAACCTAAAGTAAAATGCCCCACAGCGCTGAGTGCAT
ATAATGCATTCTCTAGTGAAAAACCTTGTTGGCATAAAAAGGCTA
ATTGATTTTCGAGAGTTTCATACTGTTTTTCTGTAGGCCGTGTAC
CTAAATGTACTTTTGCTCCATCGCGATGACTTAGTAAAGCACATC
TAAAACTTTTAGCGTTATTACGTAAAAAATCTTGCCAGCTTTCCC
CTTCTAAAGGGCAAAAGTGAGTATGGTGCCTATCTAACATCTCAA
TGGCTAAGGCGTCGAGCAAAGCCCGCTTATTTTTTACATGCCAAT
ACAATGTAGGCTGCTCTACACCTAGCTTCTGGGCGAGTTTACGGG
TTGTTAAACCTTCGATTCCGACCTCATTAAGCAGCTCTAATGCGC
TGTTAATCACTTTACTTTTATCTAATCTAGACATATTCGTTTAAT
ATCATAAATAATTTATTTTATTTTAAAATGCGCGGGTGCAAAGGT
AAGAGGTTTTATTTTAACTACCAAATGTTTTCGGAAGTTTTTTCG
CTTTTCTTTTTCTATCGTTTCTCAGACTCTCTTAGCGAAAGGGAA
AGAAGGTAAAGAAGAAAAACAAAACGCCTTTTCTTTTTTTGCACC
CGCTTTCCAAGAGAAGAAAGCCTTGTTAAATTGACTTAGTGTAAA
AGCGCAGTACTGCTTGACCATAAGAACAAAAAAATCTCTATCACT
GATAGGGATAAAGTTTGGAAGATAAAGCTAAAAGTTCTTATCTTT
GCAGTCTCCCTATCAGTGATAGAGACGAAATAAAGACATATAAAA
GAAAAGACAC.

To evaluate the performance of the different constructs and to determine the best timepoint for measuring extracellular protein accumulation in future studies, we expressed each protein from each expression plasmid and monitored bacterial growth and secretion in B. theta liquid culture for 48 hours (FIG. 1B). Despite similar growth kinetics between samples, we only observed high levels of secretion from BT_3382, and only with the strong, constitutive phage promoter/RBS pairs. BT_3769 only achieved levels of secretion above background when expression was driven by P_{BfP1E6}-RBS8, and even then the signal was more than two-fold lower than that measured for BT_3382 with the same promoter/RBS. Interestingly, P1T_{DP}-GH023 did not drive high enough protein expression to result in detectable levels of secretion for any of the three proteins, even after increasing the concentration of aTC from 100 ng/mL to 200 ng/mL (FIG. 1B). Similarly, none of the three expression constructs produced detectable levels of secreted BT_2472 in the media (FIG. 1B). While the cause is unclear, it is possible that the C-terminal domain of this protein is involved in secretion and was compromised by the 3×FLAG tag that was included for immunodetection.

For both BT_3382 and BT_3769, we observed peak extracellular protein accumulation when B. theta cultures grew to late log phase (16-20 hr, OD₆₀₀ 0.6-0.8). Beyond this point however, the amount of BT_3769 in the culture media rapidly dropped to undetectable levels within 8 hours, whereas the level of BT_3382 only dropped by ~15% when measured 48 hr later (FIG. 1B). We suspect that the persistently high levels of BT_3382 are due to higher protein stability rather than continued production and secretion by B. theta during stationery growth as BT_3382 can be enriched in OMVs, which may lead to higher thermostability.

Based on these results, we decided to 1) use PBfP1E6-RBS8 as our baseline promoter/RBS pair to drive expression of all constructs moving forward since it resulted in the highest observed secretion levels in this preliminary screen, and 2) collect all supernatant samples between late log and stationary phase of growth to ensure consistent detection of secreted products within the predicted window of protein stability for all samples.

### Example 3: Identification of Secretion Carrier Candidates from B. theta Endogenous Machinery and E. coli Exogenous Machinery

To continue developing our toolkit, we next sought to identify signal peptides, full-length proteins, or protein domains to serve as “secretion carriers” that promote extracellular export of heterologous proteins from B. theta. Typical approaches either utilize endogenous secretion machinery with homology to known systems or introduce exogenous secretion systems/tags from other bacterial strains. Because most previously characterized secretion systems in Gram-negative bacteria are either incomplete or not conserved in the B. theta genome, the endogenous secretion systems of B. theta are still poorly understood. To circumvent this limitation, we identified three secretion strategies—leaky outer membrane (OM), fusion partner, and outer membrane vesicle (OMV)—that are generally applicable for most Gram-negative bacteria (FIG. 2A) and searched for endogenous candidates within each of these categories in the B. theta genome. To ensure thorough investigation of all potential solutions for promoting secretion of heterologous proteins in B. theta, we also selected two exogenous secretion systems from Escherichia coli (E. coli)—T1 SS and T8SS (FIG. 2A), described below, and transferred these into B. theta for testing as well.

The leaky OM mechanism relies on transport of proteins into the periplasm followed by secretion into the extracellular space through natural OM leakage. We selected two B. theta leaky OM candidates for our screen: SusB, a periplasmic protein with a Sec SP of the well-studied B. theta starch utilization system (Sus), and BT_3769, which also has a Sec SP and is highly secreted. To identify native B. theta proteins that could be used as secretion carriers for the fusion partner strategy, in which heterologous cargo are fused to full-length native secretory proteins for co-transportation out of the cell, we evaluated data from a study that quantified the abundance of B. theta proteins in four separate fractions of liquid culture: inner membrane (IM), OM, OMV pellets (OMVp), and OMV-free supernatants (SUP) (45). We compared the level of each protein in the secreted (OMVp, SUP) vs unsecreted (IM+0M) fractions, revealing a list of OMVp-enriched secretory proteins, SUP-enriched secretory proteins, or proteins highly secreted in both fractions (FIG. 15). From this list, we identified 33 candidates with high abundance in the OMVp, SUP, or both fractions for use as fusion partners.

Finally, we used a similar approach to identify native B. theta proteins secreted via OMVs through a mechanism that also utilizes the Sec pathway but includes a conserved motif called the lipoprotein export signal (LES), a five-residue sequence that immediately follows the lipoprotein SP cleavage site (+2~+6) in many native OMV-enriched lipoproteins. We hypothesized that putative lipoprotein SPs with identifiable LES sequences would be able to secrete the heterologous proteins via the OMV pathway. We first identified 23 lipoproteins enriched in either the OMVp or the combined (OMVp+SUP) fractions of B. theta liquid culture (FIG. 15). We then expanded our search to explore lipoprotein SPs in B. theta with sequence similarity to those from other Bacteroides species. From this we identified BT_p548220, which has a lipoprotein SP with 31.7% homology at the amino acid level to a highly secreted B. fragilis protein, pBF9343.20c.

To ensure the highest chance of success for the exogenous secretion machinery approach, we selected two systems that both have a small genetic size, few components, and simple regulation: the hemolysin system (T1 SS) of uropathogenic E. coli (UPEC) (50) and the curli system (T8SS) from E. coli K-12 (51), which have both been used successfully for heterologous protein secretion in non-native hosts (52,53). The hemolysin system contains HlyB, HlyD, and TolC, which form the secretion channel, and HlyA, which is the cognate secreted product used to drive co-transport of protein cargo via fusion to its C-terminal domain (HlyAc) (FIG. 2A). To generate this system, we inserted the HlyB and HlyD genes into the same plasmid as the heterologous cargo (described below) under the control of a strong native B. theta constitutive promoter P_{BT1311} paired with the B. theta GH022 RBS (22) in a polycistronic format (FIG. 2B). We did not include TolC in this construct because it is conserved in most bacterial species (54) and several putative homologs exist in B. theta (BT_0671, BT_0857, and BT_2253). The curli secretion system is even simpler than the hemolysin system, consisting of only a single transport protein (CsgG) and an N-terminal fusion of the first 22 amino acids of the cognate secreted product (CsgA-N22) to the cargo protein (53) (FIG. 2A, Table S2). We generated this construct using the same format as for the hemolysin system (FIG. 2B).

To evaluate the sixty-one secretion carrier candidates identified above, we next selected a protein to serve as our standard secretion cargo for a large-scale screen. Because our goal is to enable therapeutic implementation of Bacteroides species in their natural gut environment, we selected a clinically relevant single domain antibody (sdAb) that targets Toxin A (TcdA) from Clostridioides difficile (55), a prominent and challenging gastrointestinal pathogen (56). Compared to full-length antibodies, the small size and structural simplicity of sdAbs allows them to be more easily expressed by bacteria and results in higher thermal and proteolytic stability in the harsh gut environment. For strategies using endogenous B. theta secretion carriers (leaky OM, fusion partner, OMV), we fused sdAb-TcdA to the C-terminus of each candidate SP or full-length carrier protein and included a C-terminal 3×FLAG tag for detection (FIG. 2B). We built the curli CsgA-N22-sdAb fusion the same way and inserted it into the PBT1311-GH022 CsgG construct described above (FIG. 2B). For the hemolysin construct, we fused HlyAc domain at the C-terminus of the sdAb, included the 3×FLAG tag at N-terminus of the construct, and inserted it into the PBT1311-GH022 HlyB-HlyD polycistronic plasmid (FIG. 2B).

### Example 4: Native B. theta Secretion Carriers Enable High-Level Extracellular Export of sdAb-TcdA

Of the sixty-one secretion carriers we identified and fused to the sdAb-TcdA, all constructs were successfully cloned and conjugated into B. theta except for BT_3434, which appeared to be lethal in E. coli DH5α. To determine the secretion efficiency of each of the other sixty secretion carriers, we grew B. theta conjugant cultures to late-log phase and measured the abundance of sdAb-TcdA in supernatant by dot blot (FIG. 2C). Twenty-six (43%) of the sixty secretion carriers produced visible signal and were thus considered “effective.” However, no candidate from the leaky OM (Sec SP) nor the exogenous secretion system approaches were represented in this group. These results are not entirely unexpected, as the leaky OM approach usually requires membrane-disrupting methods for full efficacy (57,58), and the E. coli T1 SS and T8SS may require unknown accessory proteins or regulators (51,59,60) that are not conserved in B. theta. Of the twenty-six effective secretion carriers, seven (27% of effective candidates, 11% of total) are full-length fusion partner proteins (six with Sec SPs and one with a lipoprotein SP) and nineteen (73% of effective candidates, 31% of total) are OMV-enriched lipoprotein SPs. The seven effective fusion partners represent only 20% of the thirty-five total fusion partners tested. The relatively low success rate of this class of secretion carrier could be improved with optimization of the fusion, e.g., truncation mutants, alternate orientations (N-, C-, or in-frame fusions), etc. (61-63). Conversely, the efficient secretion of sdAb-TcdA observed for 79% of the lipoprotein SPs (19/24) supports our hypothesis that lipoprotein SPs with LES sequences may be able to drive OMV-mediated secretion of heterologous proteins. Interestingly, previous studies in B. theta have reported that the LES region is composed of negatively charged amino acids in (45,64,65), yet we observed an enrichment for uncharged polar amino acids (N/Q/S/T) (FIG. 16) in the LES of effective lipoprotein SP sequences. These results suggest that both charged and uncharged polar amino acids in the LES region may play a role in packing lipoproteins into OMVs.

### Example 5: A Positively Charged Region and a Length-Restricted Hydrophobic Region are Provide for Effective Heterologous Protein Secretion by Lipoprotein SPs

Most of the effective secretion carriers we identified were lipoprotein SPs derived from the endogenous B. theta OMV export category, however, five B. theta lipoprotein SPs that we tested did not effectively mediate secretion of sdAb-TcdA: BT_1488, BT_1896, BT_3147, BT_3148, and BT_3383 (FIG. 2B). To determine if effective lipoprotein SPs harbor other unique features in addition to the LES, we further analyzed the amino acid sequences of our lipoprotein SP collection. These SPs are composed of a positively charged N-terminal region (n), a central hydrophobic region (h), a cysteine residue after the cleavage site (+1), and an LES motif (+2~+6) (45,64,66). Interestingly, we found that the backbones (n- and h-regions) of the five ineffective lipoprotein SPs are either very short (~10 residues) or very long (~40 residues), compared to the backbones of effective lipoprotein SPs (16-34 residues) (FIG. 3A). All five ineffective lipoprotein SPs lack a positively charged N-terminal—region and BT_3383 SP also has no clear h-region (FIG. 3B). These results suggest that, in addition to the LES, the presence of positively charged residues in the n-region (i.e., N-terminal region), as well as a minimum combined length of the n- and h-regions may be factors that drive lipoprotein SP-mediated OMV secretion in B. theta.

To test this hypothesis, we swapped the N-terminal charged and hydrophobic central regions of the five ineffective SPs with those from an effective SPs to see if we could improve their secretion efficiency through rational design. We chose the SP from BT_3630 as our standard based on its layout of charged and hydrophobic regions, which is broadly representative of the collection of lipoprotein SPs that we identified as effective (FIG. 3C). For each of the five ineffective lipoprotein SP sequences (with one exception, noted below), we made the following three SP variants and fused them to the sdAb-TcdA gene: 1) added two N-terminal lysines immediately after the start codon to introduce the positively charged region (SP-N), 2) replaced the hydrophobic region with the one from BT_3630 SP (SP-H), or 3) introduced both modifications (SP-NH) (FIG. 3C). The only variant that was not generated was the SP-N version of the BT_3383 SP; because it does not have an obvious h-region we concluded that addition of an n-region would not be sufficient to rescue secretion activity.

We observed enhanced secretion of the sdAb-TcdA only for SPs that had both an added N-terminal charged domain and a swapped hydrophobic region (SP-NH variants) (FIG. 3C), suggesting that both regions are necessary, but neither is sufficient to drive the high-level secretion in B. theta. Notably, the increase in sdAb secretion measured for the BT_3148 SP-NH variant was substantially lower than for the other SP-NH variants (FIG. 3C). Inspection of the LES sequence revealed positively charged amino acids in the +3 and +6 positions, which is consistent with previous findings that interspersed positively charged amino acids may offset the LES-mediated OMV secretion (45). These results begin to define the requirements for the presence and placement of charged and hydrophobic residues in B. theta lipoprotein SP sequences that can derive heterologous protein secretion.

### Example 6: B. theta Secretion Carriers Mediate Export of Multiple Types of Functional Protein Cargo

Toward our goal of establishing a flexible toolbox to enable efficient secretion of diverse heterologous protein cargo, we next tested the ability of the twenty-six effective secretion carriers (FIG. 2B) to secrete six additional proteins with therapeutic and/or diagnostic functions. Three of the six proteins are disease-targeting antibody fragments, including an sdAb targeting tumor necrosis factor alpha (sdAb-TNFα) (67), an antigen associated with chronic conditions such as inflammatory bowel disease (IBD) (68); another sdAb targeting epidermal growth factor receptor (sdAb-EGFR) (69), commonly overexpressed in colon cancer (70); and a single-chain variable fragment targeting human epidermal growth factor receptor-2 (scFv-HER2) (71), mainly known for its role in breast cancer, but also implicated in colon cancer (72). This set of proteins allows us to evaluate secretion efficiency across diverse antibody fragment formats while still focusing on targets relevant to gastrointestinal delivery by engineered living therapeutics. We selected the other three proteins to provide a diverse set of reporter functions: NanoLuc (Nluc) (73), enhanced green fluorescent protein (EGFP) (74), and β-lactamase (BLac) (75), which yield quantifiable outputs of luminescence, fluorescence, or colorimetric signal, respectively.

Each of these six cargo proteins were fused to each of the twenty-six secretion carriers, resulting in one hundred and fifty-six new carrier-cargo pairs. With the exception of EGFP, all cargo were effectively secreted from B. theta and accumulated at varying levels in culture supernatants (FIG. 4A). We suspect that the exceptionally low levels of EGFP detected in culture media may be due to rapid folding of this protein, which has been reported to stall the translocon secretory machinery (76). For the other cargo, we observed considerable variability in secretion efficacy both between different secretion carriers as well as amongst cargo secreted by the same carrier. In contrast to EGFP, we detected secreted Nluc at high levels across the majority of secretion carriers, suggesting that Nluc can be broadly used as a highly sensitive reporter for measuring secretion efficiency. Compared to the other proteins tested, Nluc has a higher solubility and a more acidic isoelectric point (Table 1), both of which have been reported to enhance protein secretion (77,78) and hence might account for its high-level secretion.

TABLE 1

| | Predicted | |
|---|---|---|
| | scaled | |
| | solubility | pl |
| sdAb-TcdA | 0.503 | 9.06 |
| sdAb-TNFα | 0.429 | 5.9 |
| sdAb-EGFR | 0.516 | 9.08 |
| scFv-HER2 | 0.457 | 9.3 |
| EGFP | 0.607 | 5.76 |
| Nluc | 0.581 | 5.12 |
| BLac | 0.454 | 5.53 |

Interestingly, although all sdAbs share similar structural framework (79), the three sdAbs tested here were not secreted at consistent levels by the same secretion carriers. It has been previously reported that such cargo-specific interactions with signal peptides can indirectly impact secretion by influencing other cellular processes such as protein biosynthesis, folding kinetics, and structural stability (80), which could explain some of the variability that we observed.

Finally, to verify that the secreted protein products were properly folded and not otherwise functionally disrupted by fusion to the secretion carriers, we performed functional assays to measure the antigen binding or enzymatic activity of each of the secreted cargo proteins in B. theta culture supernatants. Because the readouts of these functional assays are not equivalent across cargo (FIG. 17), we scaled the output values to a normalized range of zero to one, corresponding to the lowest and highest readout for each cargo across the twenty-six secretion carriers (FIG. 4B, lower panel). We then created a “secretion score” for each carrier by summing the normalized functional outputs for all six cargo proteins for each secretion carrier (FIG. 4B, upper panel). These results revealed several lipoprotein SPs that are broadly effective for secreting diverse cargo with BT_3630 SP and BT_3067 SP emerging as the most consistent and robust broadly active secretion carriers.

### Example 7: B. theta Secretion Carriers Function Across Multiple Bacteroides Species

Toward the goal of developing universal secretion tools for the Bacteroides genus, we next sought to evaluate the B. theta-derived secretion carriers in other Bacteroides species. We selected the ten carriers with the highest secretion scores (FIG. 4B) and measured the level of secretion (FIG. 5A) and functionality (FIG. 5B) of each of the six cargo proteins when expressed in three different Bacteroides species: B. fragilis, B. ovatus, and B. vulgatus. We were unable to generate B. fragilis transconjugants for six of the carrier-cargo pairs, which may be due to low conjugation efficiency in this species (21) or lethal intracellular aggregation of protein cargo (81). For all other Bacteroides transconjugants, the results mirrored those observed in B. theta (FIG. 4); secretion efficiency varies not only between cargo but also between species. Among the Bacteroides species tested, B. ovatus generally demonstrated the highest secretion levels for any given carrier-cargo pair, which may be due to the closer phylogenetic relationship between B. theta and B. ovatus (20). As we observed with B. theta, Nluc generally had the highest secretion levels among six cargoes across all three Bacteroides species. In contrast, efficient secretion of sdAb-EGFR and scFv-HER2 appears to be restricted to only a few selected secretion carriers.

After validating the heterologous protein secretion capacity of the B. theta-derived secretion carriers across four Bacteroides species, we next sought to quantify secretion titers in these species. For these measurements, we selected five of our seven cargo proteins: four antibody fragments and Nluc. As noted above, EGFP was not secreted (FIG. 4A) and, while BLac is highly secreted, it reaches saturation in the enzymatic assay too quickly to be suitable for quantification (FIG. 5A, 5B), thus we excluded these two reporters from this assessment. For each of the other five cargo, we identified the two secretion carriers that yielded the highest functional protein levels of each cargo in each species (FIG. 5B) and selected these cargo-carrier-species combinations for our analysis. Cultures were grown to late-log phase and the level of protein in each culture supernatant was quantified by comparison to standard curves of known concentrations of purified proteins (FIG. 5C). For the subset of secretion carriers tested, we observed secretion titers within a relatively narrow range across all four Bacteroides species for sdAb-TcdA (145-320 ng/mL) and sdAb-TNFα (65-150 ng/mL). Conversely, the secretion levels of sdAb-EGFR (180-12000 ng/mL), scFv-HER2 (10-250 ng/mL), and Nluc (33000-158000 ng/mL) were much more variable between secretion carriers, Bacteroides species, or both.

### Example 8: Modified Inducible Expression System Yields Enhanced Protein Secretion

Having successfully established an approach to enable heterologous protein secretion from B. theta and other Bacteroides species, we next sought to engineer additional layers of flexibility, control, and enhancement using an inducible gene expression system. In our initial studies, we observed that the aTc-inducible P2-A21-tetR-P1T_{DP}-GH023 expression cassette resulted in much lower secretion than P_{BfP1E6}-RBS8 (FIG. 1B), presumably due to lower expression. To generate an inducible system capable of achieving much higher expression levels, and thus much higher secretion levels, we introduced two modifications aimed at enhancing activity. First, we replaced the GH023 RBS with the A21 RBS (FIG. 6A), which is the strongest RBS tested amongst a collection of Bacteroides RBS sequences when paired with the P1T_{DP} promoter (20). Because the original construct already contained an A21 RBS sequence (FIG. 1A), we replaced the TetR-driving P2-A21 promoter/RBS with P_{BT1311} and its native RBS (FIG. 6A) to avoid issues such as unwanted homologous recombination events between identical RBS sequences (82). The P_{BT1311} sequence with its native RBS sequence is shown below:

(SEQ ID NO: 199)
TGATCTGGAAGAAGCAATGAAAGCTGCTGTTAAGTCTCCGAATCA
GGTATTGTTCCTGACAGGTGTATTCCCATCCGGTAAACGCGGATA
CTTTGCAGTTGATCTGACTCAGGAATAAATTATAAATTAAGGTAA
GAAGATTGTAGGATAAGCTAATGAAATAGAAAAAGGATGCCGTCA
CACAACTTGTCGGCATTCTTTTTTGTTTTATTAGTTGAAAATATA
GTGAAAAAGTTGCCTAAATATGTATGTTAACAAATTATTTGTCGT
AACTTTGCACTCCAAATCTGTTTTTAAAGA.

The A21 RBS sequence is shown below:

(SEQ ID NO: 200)
CGCATTTTAAAATAAAATAAATTATTTATGATATTAAACGAAT.

The P1T_{DP}-A21 (promoter+RBS) sequence is shown below:

(SEQ ID NO: 201)
TTTGCACCCGCTTTCCAAGAGAAGAAAGCCTTGTTAAATTGACTT
AGTGTAAAAGCGCAGTACTGCTTGACCATAAGAACAAAAAAATCT
CTATCACTGATAGGGATAAAGTTTGGAAGATAAAGCTAAAAGTTC
TTATCTTTGCAGTCTCCCTATCAGTGATAGAGACGCATTTTAAAA
TAAAATAAATTATTTATGATATTAAACGAAT.

To measure the activity of our enhanced inducible expression system, we fused Nluc with the high-efficiency secretion carrier BT_3630 SP and generated two expression/secretion constructs: one driven by the aTc-inducible P1T_{DP}-A21 promoter, and one driven by the high-activity constitutive P_{BfP1E6}-RBS8 as both a positive control and reference point for high-level expression (FIG. 6B, top). To quantify secretion, we induced freshly diluted overnight cultures with aTc and performed bioluminescence measurements from 8 to 48 hr post-induction. Induction of Nluc expression from the enhanced P1T_{DP}-A21 promoter resulted in secretion levels up to 2300-fold higher than uninduced controls, and over 3-fold higher than the P_{BfP1E6}-RBS8 constitutively expressed control (FIG. 6B, top). Because the B. theta cultures demonstrated a period of initial slow growth following a 1:100 dilution of the overnight cultures into fresh induction medium, we repeated the experiment using cultures diluted at 1:10. These samples achieved late-log phase growth after only 12 hr, compared to nearly 30 hr for the samples diluted at 1:100, but reached nearly the same maximal secretion levels (FIG. 6B, bottom), which suggests that the dilution factor does not affect the performance of this promoter. To verify the portability of this modified promoter, we repeated the experiments in all four different Bacteroides species and observed that the enhanced inducible expression system yields similar levels of secreted Nluc, and similar fold-induction levels compared to uninduced samples, across all four species tested (FIG. 6C). Interestingly, for B. fragilis and B. vulgatus, this promoter gave rise to slightly lower secretion levels in induced cultures and slightly higher apparent expression leakage in uninduced cultures, resulting in overall lower fold-induction levels for these two species. This difference, similar to what we observed for overall secretion carrier performance across Bacteroides species (FIG. 5), may also be linked to their evolutionary distance from the other two species.

### Example 9: Different Secretion Carriers Mediate Distinct Post-Secretion Extracellular Fate of Protein Cargo

Toward our goal of reproducibly delivering therapeutic proteins into specific physiological niches such as the gut lumen, we next sought to investigate the post-secretion extracellular fate of heterologous proteins exported using our platform. Because we expect OMV associated proteins to have fundamentally different characteristics than freely soluble proteins, such as thermostability, protease resistance, bioavailability, and dissemination to other body sites (83), precise determination of the extracellular destination mediated by different secretion carriers is required to fully understand and optimize our platform. Based on the high secretion levels and high sensitivity observed in earlier experiments, we selected Nluc as the secretion cargo for these studies. From our collection of twenty-six effective secretion carriers (FIG. 2B), we selected four candidates with diverse structures and high efficiency of Nluc secretion (FIG. 4B) across fusion partner and OMV categories for further investigation: BT_0169 and BT_0569 (full-length fusion partners with Sec SPs); BT_0922 (full-length fusion partner with lipoprotein SP); BT_3630 SP (lipoprotein SP).

To determine the extracellular fate of Nluc when secreted by these four carriers, we grew late-log phase liquid cultures of B. theta expressing each carrier-Nluc fusion, separated the cell pellets from the total supernatants, then further separated the supernatants into the soluble and OMV fractions. Following concentration the OMV fraction by twenty-fold, we measured the Nluc protein abundance (FIG. 7A) and luminescence activity (FIG. 7B) in each fraction. Consistent with the differences in carrier-specific Nluc secretion efficiency observed in earlier experiments (FIG. 4), western analysis revealed that the majority of the BT_0169-Nluc and BT_0569-Nluc was retained in the cell pellets, whereas BT_0922-Nluc and BT_3630 SP-Nluc were mostly secreted, although with different abundances in different fractions. We observed that BT_0922-Nluc appeared to have been cleaved, showing a faint band at its expected molecular weight of ~60 kDa in the cell pellet fraction. A smaller product was detected at ~23 kDa in all other BT_0922-Nluc culture fractions. In luciferase assay, we found that soluble fraction of BT_0169-Nluc still account for ~80% luminescence of total supernatant after the OMVs were separated (FIG. 7B), suggesting that BT_0169-Nluc was mainly secreted in soluble form. For BT_0569-Nluc, BT_0922-Nluc, and BT_3630 SP-Nluc, they were secreted evenly in soluble form and OMV because their soluble fraction account for ~50% of total supernatant after OMVs were separated. However, when we observed the luminescence of 20-fold concentrated OMV fractions, BT_0569 produced significantly higher proportion of OMV-associated Nluc compared to BT_0169, BT_0922, and BT_3630 SP. Remarkably, BT_3630 SP-Nluc showing in both soluble and OMV fractions suggests that lipoprotein SPs secrete the heterologous proteins through not only OMV but also OMV-independent pathways.

Because the luminescence activity assay to quantify secreted proteins in the OMV fraction cannot differentiate between surface-anchored and intra-vesicular Nluc (FIG. 7D), we next investigated the OMV-specific localization of two carrier-Nluc fusions to further refine our ability to precisely implement therapeutic protein delivery with our engineered platform. We selected BT_0569 (fusion partner with Sec SP) and BT_3630 SP (lipoprotein SP) as the secretion carriers for this study, based on the prediction that they would send cargo to the OMV lumen or surface, respectively. We performed a proteinase K accessibility assay on total OMV fractions isolated from liquid cultures of B. theta expressing BT_0569-Nluc and BT_3630 SP-Nluc (FIG. 7C). BT_0569-Nluc was highly resistant to degradation at both early (5 min) and late (30 min) timepoints across nearly all proteinase K concentrations tested, whereas BT_3630 SP-Nluc was much more sensitive to degradation at the higher proteinase K concentrations and over time. These results agree with our prediction that a Sec SP (BT_0569) and a lipoprotein SP (BT_3630) would mediate packaging of protein cargo in the lumen or onto the surface of OMVs, respectively. Together these results suggest that the post-secretion fate (soluble, OMV surface, or OMV lumen) of protein cargo can be controlled by selection of secretion carriers (FIG. 7D), which will allow more refined customization of Bacteroides-based in situ delivery systems for specific applications.

### Example 10: Probing the Size Limit of Lipoprotein SP-Mediated Protein Secretion

To fully explore the capacity of B. theta for in situ delivery of protein-based therapeutics, we next wanted to determine if there is a limit on the size of the protein cargo that can be secreted by lipoprotein SPs with high secretion scores (FIG. 4B). We therefore selected BT_3630 SP as our representative SP and fused it to seven different cargo proteins—three endogenous B. theta proteins and four heterologous proteins—selected to cover a broad range of molecular weight: Nluc (25 kDa), cellulase (Cel; 41 kDa), BT_3686 (53 kDa), chitinase (ChiA; 68 kDa), BT_3703 (SusB; 88 kDa), β-galactosidase (LacZ; 122 kDa), and BT_3169 (148 kDa) (FIG. 7E). All constructs were fused with a C-terminal 3×FLAG tag to enable immunodetection. To minimize the metabolic burden of high-level protein expression, we used the aTc-inducible P1T_{DP}-A21 promoter instead of the constitutive P_{BfP1E6}-RBS8. Following growth in liquid culture to late log phase, we analyzed the culture supernatants for the presence of each secreted protein and only observed extracellular accumulation of the four smallest cargo. The next largest protein cargo, SusB, was clearly observed in the pellet but not in the supernatant, suggesting an apparent molecular weight cutoff between 68 and 88 kDa (FIG. 7F). While it is possible that the observed low secretion levels of the two largest constructs are a result of low expression levels, the results observed for SusB suggest the existence of a potential protein size limit for lipoprotein SP-mediated secretion.

### Example 11: Secretion Carriers Mediate In Situ Delivery of Heterologous Proteins from B. theta in the Mouse Gut

Finally, to validate the in vivo functionality of our in vitro-characterized B. theta secretion carriers, we next investigated their performance in the gastrointestinal tract of mice. Following pre-treatment with an antibiotic cocktail, we inoculated C57Bl/6 mice with: B. theta constitutively expressing Nluc with no secretion carrier (intracellular), B. theta constitutively expressing Nluc fused with BT_0294 SP (secreted; highest efficiency in secreting Nluc (FIGS. 17 and 5C)), wild-type (WT) B. theta (no expression control), or PBS (no treatment control) (FIG. 8A). We monitored general health (mouse weight), B. theta colonization (colony forming units [CFU] in feces), and Nluc activity (luminescence) for two months (FIG. 8). We observed no difference in weight between any group, suggesting that our engineered strains had no obvious adverse effects on mouse health (FIG. 8B). Both the intracellular and secreted Nluc strains engrafted and persisted at identical levels (FIG. 8C), demonstrating robust, long-term colonization despite competition with the native (antibiotic treated) mouse microbiota. Because we performed these studies in antibiotic-treated conventional mice rather than germ-free, we used selective plating to isolate and quantify fecal CFU of our strains, which harbor erythromycin-resistance markers. Thus, the CFU of the WT strain, which has no erythromycin-resistance marker, could not be quantified using this approach (FIG. 8C). Luminescence activity for both intracellular and secreted Nluc was readily detectable in the feces over the entire experimental time course, indicating that the secreted cargo was not only continuously present but also functional. The presence of Nluc in the fecal pellets of the mice colonized with the non-secreting (intracellularly expressing) B. theta strain suggests that some amount of cell lysis occurred in vivo in the mouse intestine or ex vivo during sample processing; however, the luminescence values measurements for fecal samples from mice colonized with B. theta expressing BT_0294 SP-Nluc (secreted) were around ten-fold higher than the intracellular Nluc values throughout the experimental timecourse. Despite stable B. theta colonization (FIG. 8C), we observed a slow decrease in luminescence activity over time for both the intracellular and secreted Nluc variants (FIG. 8D). This is not entirely unexpected, as the expression constructs were not genome-integrated, and no antibiotic selection pressure was applied to maintain the plasmids in the B. theta cells.

### Example 12

To further investigate the factors determining the secretion efficiency of lipoprotein secretion carriers, four (BT_0294SP; BT_3630SP; BT_3740; and BT_3741 SP) lipoprotein secretion proteins with diverse n-, h-, and LES regions were selected for domain shuffling. See FIG. 9. 64 chimera were generated with an Nluc reporter. The n-regions were MRNLK (SEQ ID NO:150) MMKK (SEQ ID NO:151), MNYSCRK (SEQ ID NO:152), and MK. The h-regions were: WLYACSLAIAFGVLSFVTVS (SEQ ID NO:139), GILFVLTAAFLAS (SEQ ID NO:146), TIVPIIIGTLLSGA (SEQ ID NO:147), and MLRIIMILLGALLLTN (SEQ ID NO:145). The LES regions were CHDDDD (SEQ ID NO:154), CQQEEN (SEQ ID NO:155), CSNDEP (SEQ ID NO:156), and CSGDFE (SEQ ID NO:157). Nluc secretion efficiency was then tested.

The results are shown in FIG. 10. the luminescence readouts of all 64 chimera lipoprotien SPs by n-, h-, or LES domains were grouped, revealing LES domain is the major determinant of cargo secretion efficiency of lipoprotein SPs. That is, as long as a secretion carrier meets the basic requirement (n-region has at least +1 charge and the h-region is longer than 13aa), then the secretion efficiency of that secretion carrier is determined by the amino acid composition of LES.

An optimal lipoprotein SP backbone for building LES library was investigated. Some backbones and LES of secretion carriers might have synergistic effects on secretion efficiency. In order to find a lipoprotein SP backbone that can most faithfully reflect the secretion efficiency of LES, 7 lipoprotein SPs were chosen as candidates. Their n-/h- (backbone) domain were swapped with each other. See FIG. 11. The backbones (n-region underlined were MRNLKWLYACSLAIAFGVLSFVTVS (SEQ ID NO:163), MMKKTILLTSIIAIAIVSMLSS (SEQ ID NO:164), MKLRIYTLLIAFCAAWSLHS (SEQ ID NO:165), MNKKFLSVILFGALMTVSTGTFVS (SEQ ID NO:166), MKKFFYLSALSLGMMCSITA (SEQ ID NO:167), MRKEKLYTGCLLLMALITGS (SEQ ID NO:168), and MKMLRIIMILLGALLLTN (SEQ ID NO:169). The LES regions were CHDDDD (SEQ ID NO:154), CDSEKD (SEQ ID NO:158), CDNDDD (SEQ ID NO:159), CKDYDD (SEQ ID NO:160), CSDDDT (SEQ ID NO:161), CSEEEN (SEQ ID NO:162), and CSGDFE (SEQ ID NO:157). See FIG. 12. The secretion efficiency of different LES was found to be closest to their average secretion efficiency when fusing to the backbone of BT_2479 SP. The BT_2479 SP backbone can most faithfully reflect the average secretion efficiency of different LESs. Therefore, BT2479 SP backbone was selected for building an LES library. See FIG. 13A.

Based on the consensus amino acid pattern of LES of high-/low-secretion native lipoprotein SPs of B. theta, we hypothesized that if there are more uncharged/negatively charged residues in LES domain (+2~+6), then the secretion efficiency of that lipoprotein SP would be higher. To create LES library that can fine-tune the secretion efficiency of lipoprotein SPs, we tried to include both negatively, positively charged residues and uncharged residues for each position to build up the LES library. About 7,680 colonies was handpicked into eighty 96-well plate for screening. Luciferase assays were performed to measure the Nluc levels in supernatants. Colonies that generated a broad range of luminescence were selected for sequencing. The sequencing results were in line with the hypothesis. Overall, we found more S/N/D/E in high efficiency secretion carriers and more G/K in low-efficiency secretion carriers. See FIG. 14.

Additionally, 25 BT_2479 SP-LES variants with 4 protein cargoes (sdAb-TcdA, sdAb-EGFR, Nluc, and human I110 (hIL10)). The secretion levels of the variants were examined. After doing the cargo-wise scaling for the readouts, the secretion score was calculated for each BT_2479 SP-LES variant, which can be a toolkit for fine-tuning the secretion efficiency of lipoprotein SPs. A strong correlation between the net charge of LES and the secretion score was identified. See FIG. 18.

The secretion carriers developed herein enable enhanced heterologous protein secretion across multiple Bacteroides species. By establishing a toolbox enabling the secretion of biotherapeutic proteins from permanently colonizing Bacteroides strains, we provide a means to utilize the living therapeutics platform for a broader range of diseases, including chronic conditions that require continuous treatment. Our additional characterization of the secretion carriers that we identified also provides a means for downstream users to select or engineer secretion carriers that are best suited for their particular goals and applications. Beyond therapeutic applications, Bacteroides species are prominent and abundant representative members of the gut microbiota (14); the secretion tools described here could be useful for studying interspecies interactions and microbiota-host crosstalk in the gut.

| | Three | One |
|---|---|---|
| | letter | letter |
| Amino acid | code | code |
| alanine | ala | A |
| arginine | arg | R |
| asparagine | asn | N |
| aspartic acid | asp | D |
| asparagine or aspartic acid | asx | B |
| cysteine | cys | C |
| glutamic acid | glu | E |
| glutamine | gln | Q |
| glutamine or glutamic acid | glx | Z |
| glycine | gly | G |
| histidine | his | H |
| isoleucine | ile | I |
| leucine | leu | L |
| lysine | lys | K |
| methionine | met | M |
| phenylalanine | phe | F |
| proline | pro | P |
| serine | ser | S |
| threonine | thr | T |
| tryptophan | trp | W |
| tyrosine | tyr | Y |
| valine | val | V |

### REFERENCES

1. Kelly V W, Liang B K, Sirk S J. Living Therapeutics: The Next Frontier of Precision Medicine. ACS Synth Biol. 2020 Dec. 18; 9(12):3184-201.
2. Pedrolli D B, Ribeiro N v., Squizato P N, de Jesus V N, Cozetto D A, Tuma R B, et al. Engineering Microbial Living Therapeutics: The Synthetic Biology Toolbox. Trends Biotechnol. 2019 January; 37(1):100-15.
3. Isabella V M, Ha B N, Castillo M J, Lubkowicz D J, Rowe S E, Millet Y A, et al. Development of a synthetic live bacterial therapeutic for the human metabolic disease phenylketonuria. Nat Biotechnol. 2018 Oct. 1; 36(9):857-64.
4. Chen Z, Guo L, Zhang Y, L. Walzem R, Pendergast J S, Printz R L, et al. Incorporation of therapeutically modified bacteria into gut microbiota inhibits obesity. Journal of Clinical Investigation. 2014 Aug. 1; 124(8):3391-406.
5. Somabhai C A, Raghuvanshi R, Nareshkumar G. Genetically Engineered Escherichia coli Nissle 1917 Synbiotics Reduce Metabolic Effects Induced by Chronic Consumption of Dietary Fructose. PLoS One. 2016 Oct. 19; 11(10):e0164860.
6. Charbonneau M R, Isabella V M, Li N, Kurtz C B. Developing a new class of engineered live bacterial therapeutics to treat human diseases. Nat Commun. 2020 Dec. 8; 11(1):1738.
7. Steidler L, Hans W, Schotte L, Neirynck S, Obermeier F, Falk W, et al. Treatment of Murine Colitis by Lactococcus lactis Secreting Interleukin-10. Science (1979). 2000 Aug. 25; 289(5483):1352-5.
8. Ho C L, Tan H Q, Chua K J, Kang A, Lim K H, Ling K L, et al. Engineered commensal microbes for diet-mediated colorectal-cancer chemoprevention. Nat Biomed Eng. 2018 Jan. 10; 2(1):27-37.
9. Zhang Y, Zhang Y, Xia L, Zhang X, Ding X, Yan F, et al. Escherichia coli Nissle 1917 Targets and Restrains Mouse B16 Melanoma and 4T1 Breast Tumors through Expression of Azurin Protein. Appl Environ Microbiol. 2012 November; 78(21):7603-10.
10. Palmer J D, Piattelli E, McCormick B A, Silby M W, Brigham C J, Bucci V. Engineered Probiotic for the Inhibition of Salmonella via Tetrathionate-Induced Production of Microcin H47. ACS Infect Dis. 2018 Jan. 12; 4(1):39-45.
11. Hwang I Y, Koh E, Wong A, March J C, Bentley W E, Lee Y S, et al. Engineered probiotic Escherichia coli can eliminate and prevent Pseudomonas aeruginosa gut infection in animal models. Nat Commun. 2017 Apr. 11; 8(1):15028.
12. The Human Microbiome Project Consortium. Structure, function and diversity of the healthy human microbiome. Nature. 2012 Jun. 13; 486(7402):207-14.
13. Nishijima S, Suda W, Oshima K, Kim S W, Hirose Y, Morita H, et al. The gut microbiome of healthy Japanese and its microbial and functional uniqueness. DNA Research. 2016 April; 23(2):125-33.
14. Wexler A G, Goodman A L. An insider's perspective: Bacteroides as a window into the microbiome. Nat Microbiol. 2017 May 25; 2(5):17026.
15. Faith J J, Guruge J L, Charbonneau M, Subramanian S, Seedorf H, Goodman A L, et al. The Long-Term Stability of the Human Gut Microbiota. Science (1979). 2013 Jul. 5; 341(6141).
16. Horn N, Carvalho A L, Overweg K, Wegmann U, Carding S R, Stentz R. A Novel Tightly Regulated Gene Expression System for the Human Intestinal Symbiont Bacteroides thetaiotaomicron. Front Microbiol. 2016 Jul. 13; 7.
17. Taketani M, Zhang J, Zhang S, Triassi A J, Huang Y J, Griffith L G, et al. Genetic circuit design automation for the gut resident species Bacteroides thetaiotaomicron. Nat Biotechnol. 2020 Aug. 1; 38(8):962-9.
18. Zheng L, Tan Y, Hu Y, Shen J, Qu Z, Chen X, et al. CRISPR/Cas-Based Genome Editing for Human Gut Commensal Bacteroides Species. ACS Synth Biol. 2022 Jan. 21; 11(1):464-72.
19. Garcia-Bayona L, Comstock L E. Streamlined Genetic Manipulation of Diverse Bacteroides and Parabacteroides Isolates from the Human Gut Microbiota. mBio. 2019 Aug. 27; 10(4).
20. Lim B, Zimmermann M, Barry N A, Goodman A L. Engineered Regulatory Systems Modulate Gene Expression of Human Commensals in the Gut. Cell. 2017 April; 169(3):547-558.e15.
21. Whitaker W R, Shepherd E S, Sonnenburg J L. Tunable Expression Tools Enable Single-Cell Strain Distinction in the Gut Microbiome. Cell. 2017 April; 169(3):538-546.e12.
22. Mimee M, Tucker A C, Voigt C A, Lu T K. Programming a Human Commensal Bacterium, Bacteroides thetaiotaomicron, to Sense and Respond to Stimuli in the Murine Gut Microbiota. Cell Syst. 2015 July; 1(1):62-71.
23. Hamady Z Z R, Scott N, Farrar M D, Lodge J P A, Holland K T, Whitehead T, et al. Xylan-regulated delivery of human keratinocyte growth factor-2 to the inflamed colon by the human anaerobic commensal bacterium Bacteroides ovatus. Gut. 2010 Apr. 1; 59(4):461-9.
24. Jones D R, Smith M B, McLean R, Grondin J M, Amundsen C R, Inglis G D, et al. Engineering dual-glycan responsive expression systems for tunable production of heterologous proteins in Bacteroides thetaiotaomicron. Sci Rep. 2019 Dec. 22; 9(1):17400.
25. Wegmann U, Carvalho A L, Stocks M, Carding S R. Use of genetically modified bacteria for drug delivery in humans: Revisiting the safety aspect. Sci Rep. 2017 May 23; 7(1):2294.
26. Shepherd E S, DeLoache W C, Pruss K M, Whitaker W R, Sonnenburg J L. An exclusive metabolic niche enables strain engraftment in the gut microbiota. Nature. 2018 May 9; 557(7705):434-8.
27. Riglar D T, Silver P A. Engineering bacteria for diagnostic and therapeutic applications. Nat Rev Microbiol. 2018 Apr. 5; 16(4):214-25.
28. Sieow B F L, Wun K S, Yong W P, Hwang I Y, Chang M W. Tweak to Treat: Reprograming Bacteria for Cancer Treatment. Trends Cancer. 2021 May; 7(5):447-64.
29. Anne J, Economou A, Bernaerts K. Protein Secretion in Gram-Positive Bacteria: From Multiple Pathways to Biotechnology. In 2016. p. 267-308.
30. Schneewind O, Missiakas D M. Protein secretion and surface display in Gram-positive bacteria. Philosophical Transactions of the Royal Society B: Biological Sciences. 2012 Apr. 19; 367(1592):1123-39.
31. Costa T R D, Felisberto-Rodrigues C, Meir A, Prevost M S, Redzej A, Trokter M, et al. Secretion systems in Gram-negative bacteria: structural and mechanistic insights. Nat Rev Microbiol. 2015 Jun. 15; 13(6):343-59.
32. Trivedi A, Gosai J, Nakane D, Shrivastava A. Design Principles of the Rotary Type 9 Secretion System. Front Microbiol. 2022 May 10; 13.
33. Eom G T, Lee S H, Oh Y H, Choi J E, Park S J, Song J K. Efficient extracellular production of type I secretion pathway-dependent Pseudomonas fluorescens lipase in recombinant Escherichia coli by heterologous ABC protein exporters. Biotechnol Lett. 2014 Oct. 15; 36(10):2037-42.
34. Lynch J P, González-Prieto C, Reeves A Z, Bae S, Powale U, Godbole N P, et al. Engineered Escherichia coli for the in situ secretion of therapeutic nanobodies in the gut. Cell Host Microbe. 2023 April; 31(4):634-649.e8.
35. Bartra S S, Jackson M W, Ross J A, Plano G V. Calcium-Regulated Type III Secretion of Yop Proteins by an Escherichia coli hha Mutant Carrying a Yersinia pestis pCD1 Virulence Plasmid. Infect Immun. 2006 February; 74(2):1381-6.
36. Akeda Y, Kimura T, Yamasaki A, Kodama T, Iida T, Honda T, et al. Functional cloning of Vibrio parahaemolyticus type III secretion system 1 in Escherichia coli K-12 strain as a molecular syringe. Biochem Biophys Res Commun. 2012 October; 427(2):242-7.
37. Zafar H, Saier M H. Comparative genomics of transport proteins in seven Bacteroides species. PLoS One. 2018 Dec. 5; 13(12):e0208151.
38. Abby S S, Cury J, Guglielmini J, Neron B, Touchon M, Rocha E P C. Identification of protein secretion systems in bacterial genomes. Sci Rep. 2016 Mar. 16; 6(1):23080.
39. Farrar M D, Whitehead T R, Lan J, Dilger P, Thorpe R, Holland K T, et al. Engineering of the gut commensal bacterium Bacteroides ovatus to produce and secrete biologically active murine interleukin-2 in response to xylan. J Appl Microbiol. 2005 May; 98(5):1191-7.
40. Hamady Z Z R, Scott N, Farrar M D, Lodge J P A, Holland K T, Whitehead T, et al. Xylan-regulated delivery of human keratinocyte growth factor-2 to the inflamed colon by the human anaerobic commensal bacterium Bacteroides ovatus. Gut. 2010 Apr. 1; 59(4):461-9.
41. Hamady Z Z R, Scott N, Farrar M D, Wadhwa M, Dilger P, Whitehead T R, et al. Treatment of colitis with a commensal gut bacterium engineered to secrete human tgf-β1 under the control of dietary xylan. Inflamm Bowel Dis. 2011 September; 17(9):1925-35.
42. Carvalho A L, Fonseca S, Miquel-Clopés A, Cross K, Kok K S, Wegmann U, et al. Bioengineering commensal bacteria-derived outer membrane vesicles for delivery of biologics to the gastrointestinal and respiratory tract. J Extracell Vesicles. 2019 Dec. 1; 8(1):1632100.
43. FLORENTINE R J, KUAN-T A LUT, K M M. ENGINEERED BACTEROIDES OUTER MEMBRANE VESICLES [Internet]. WO: MASSACHUSETTS INST TECHNOLOGY; 2017. Available from: https://lens.org/089-151-059-319-997
44. Burdette L A, Leach S A, Wong H T, Tullman-Ercek D. Developing Gram-negative bacteria for the secretion of heterologous proteins. Microb Cell Fact. 2018 Dec. 20; 17(1):196.
45. Valguarnera E, Scott N E, Azimzadeh P, Feldman M F. Surface Exposure and Packing of Lipoproteins into Outer Membrane Vesicles Are Coupled Processes in Bacteroides. mSphere. 2018 Dec. 26; 3(6).
46. Li R, Liu Q. Engineered Bacterial Outer Membrane Vesicles as Multifunctional Delivery Platforms. Front Mater. 2020 Jul. 10; 7.
47. Zingl F G, Leitner D R, Thapa H B, Schild S. Outer membrane vesicles as versatile tools for therapeutic approaches. microLife. 2021 Jun. 11; 2.
48. Foley M H, Cockburn D W, Koropatkin N M. The Sus operon: a model system for starch uptake by the human gut Bacteroidetes. Cellular and Molecular Life Sciences. 2016 Jul. 2; 73(14):2603-17.
49. Wilson M M, Anderson D E, Bernstein H D. Analysis of the Outer Membrane Proteome and Secretome of Bacteroides fragilis Reveals a Multiplicity of Secretion Mechanisms. PLoS One. 2015 Feb. 6; 10(2):e0117732.
50. Thomas S, Holland I B, Schmitt L. The Type 1 secretion pathway—The hemolysin system and beyond. Biochimica et Biophysica Acta (BBA)—Molecular Cell Research. 2014 August; 1843(8):1629-41.
51. Bhoite S, van Gerven N, Chapman M R, Remaut H. Curli Biogenesis: Bacterial Amyloid Assembly by the Type VIII Secretion Pathway. EcoSal Plus. 2019 Feb. 6; 8(2).
52. Garmory H S, Titball R W, Griffin K F, Hahn U, Böhm R, Beyer W. Salmonella enterica Serovar Typhimurium Expressing a Chromosomally Integrated Copy of the Bacillus anthracis Protective Antigen Gene Protects Mice against an Anthrax Spore Challenge. Infect Immun. 2003 July; 71(7):3831-6.
53. Goosens V J, Walker K T, Aragon S M, Singh A, Senthivel V R, Dekker L, et al. Komagataeibacter Tool Kit (KTK): A Modular Cloning System for Multigene Constructs and Programmed Protein Secretion from Cellulose Producing Bacteria. ACS Synth Biol. 2021 Dec. 17; 10(12):3422-34.
54. Su L, Chen S, Yi L, Woodard R W, Chen J, Wu J. Extracellular overexpression of recombinant Thermobifida fusca cutinase by alpha-hemolysin secretion system in E. coli BL21(DE3). Microb Cell Fact. 2012 Dec. 12; 11(1):8.
55. Hussack G, Arbabi-Ghahroudi M, van Faassen H, Songer J G, Ng K K S, MacKenzie R, et al. Neutralization of Clostridium difficile Toxin A with Single-domain Antibodies Targeting the Cell Receptor Binding Domain. Journal of Biological Chemistry. 2011 March; 286(11):8961-76.
56. Song J H, Kim Y S. Recurrent Clostridium difficile Infection: Risk Factors, Treatment, and Prevention. Gut Liver. 2019 Jan. 15; 13(1):16-24.
57. Wurm D J, Slouka C, Bosilj T, Herwig C, Spadiut O. How to trigger periplasmic release in recombinant Escherichia coli: A comparative analysis. Eng Life Sci. 2017 February; 17(2):215-22.
58. Chen Z, Cao J, Xie L, Li X, Yu Z, Tong W. Construction of leaky strains and extracellular production of exogenous proteins in recombinant Escherichia coli. Microb Biotechnol. 2014 Jul. 30; 7(4):360-70.
59. Khambhati K, Patel J, Saxena V, A P, Jain N. Gene Regulation of Biofilm-Associated Functional Amyloids. Pathogens. 2021 Apr. 19; 10(4):490.
60. Alav I, Kobylka J, Kuth M S, Pos K M, Picard M, Blair J M A, et al. Structure, Assembly, and Function of Tripartite Efflux and Type 1 Secretion Systems in Gram-Negative Bacteria. Chem Rev. 2021 May 12; 121(9):5479-596.
61. Sevastsyanovich Y R, Leyton D L, Wells T J, Wardius C A, Tveen-Jensen K, Morris F C, et al. A generalised module for the selective extracellular accumulation of recombinant proteins. Microb Cell Fact. 2012 Dec. 28; 11(1):69.
62. Hersch S J, Lam L, Dong T G. Engineered Type Six Secretion Systems Deliver Active Exogenous Effectors and Cre Recombinase. mBio. 2021 Aug. 31; 12(4).
63. Jarchau T, Chakraborty T, Garcia F, Goebel W. Selection for transport competence of C-terminal polypeptides derived from Escherichia coli hemolysin: the shortest peptide capable of autonomous HlyB/HlyD-dependent secretion comprises the C-terminal 62 amino acids of HlyA. Mol Gen Genet. 1994 January; 245(1):53-60.
64. Sartorio M G, Pardue E J, Scott N E, Feldman M F. Human gut bacteria tailor extracellular vesicle cargo for the breakdown of diet- and host-derived glycans. Proceedings of the National Academy of Sciences. 2023 Jul. 4; 120(27).
65. Sartorio M G, Pardue E J, Scott N E, Feldman M F. Human gut bacteria tailor extracellular vesicle cargo for the breakdown of diet- and host-derived glycans. Proceedings of the National Academy of Sciences. 2023 Jul. 4; 120(27).
66. Lauber F, Cornelis G R, Renzi F. Identification of a New Lipoprotein Export Signal in Gram-Negative Bacteria. mBio. 2016 Nov. 2; 7(5).
67. Beirnaert E, Desmyter A, Spinelli S, Lauwereys M, Aarden L, Dreier T, et al. Bivalent Llama Single-Domain Antibody Fragments against Tumor Necrosis Factor Have Picomolar Potencies due to Intramolecular Interactions. Front Immunol. 2017 Jul. 31; 8.
68. Gareb B, Otten A T, Frijlink H W, Dijkstra G, Kosterink J G W. Review: Local Tumor Necrosis Factor-a Inhibition in Inflammatory Bowel Disease. Pharmaceutics. 2020 Jun. 11; 12(6):539.
69. Roovers R C, Vosjan M J W D, Laeremans T, el Khoulati R, de Bruin R C G, Ferguson K M, et al. A biparatopic anti-EGFR nanobody efficiently inhibits solid tumour growth. Int J Cancer. 2011 Oct. 15; 129(8):2013-24.
70. Krasinskas A M. EGFR Signaling in Colorectal Carcinoma. Patholog Res Int. 2011 Feb. 14; 2011:1-6.
71. Kelly V W, Sirk S J. Short FcRn-Binding Peptides Enable Salvage and Transcytosis of scFv Antibody Fragments. ACS Chem Biol. 2022 Feb. 18; 17(2):404-13.
72. Iqbal N, Iqbal N. Human Epidermal Growth Factor Receptor 2 (HER2) in Cancers: Overexpression and Therapeutic Implications. Mol Biol Int. 2014 Sep. 7; 2014:1-9.
73. Hall M P, Unch J, Binkowski B F, Valley M P, Butler B L, Wood M G, et al. Engineered Luciferase Reporter from a Deep Sea Shrimp Utilizing a Novel Imidazopyrazinone Substrate. ACS Chem Biol. 2012 Nov. 16; 7(11):1848-57.
74. Heim R, Tsien R Y. Engineering green fluorescent protein for improved brightness, longer wavelengths and fluorescence resonance energy transfer. Current Biology. 1996 February; 6(2):178-82.
75. Mueller K E, Fields K A. Application of P-Lactamase Reporter Fusions as an Indicator of Effector Protein Secretion during Infections with the Obligate Intracellular Pathogen Chlamydia trachomatis. PLoS One. 2015 Aug. 10; 10(8):e0135295.
76. Lenders M H H, Weidtkamp-Peters S, Kleinschrodt D, Jaeger K E, Smits S H J, Schmitt L. Directionality of substrate translocation of the hemolysin A Type I secretion system. Sci Rep. 2015 Jul. 27; 5:12470.
77. Cheng C M, Tzou S C, Zhuang Y H, Huang C C, Kao C H, Liao K W, et al. Functional Production of a Soluble and Secreted Single-Chain Antibody by a Bacterial Secretion System. PLoS One. 2014 May 13; 9(5):e97367.
78. Byun H, Park J, Kim S C, Ahn J H. A lower isoelectric point increases signal sequence-mediated secretion of recombinant proteins through a bacterial ABC transporter. Journal of Biological Chemistry. 2017 December; 292(48):19782-91.
79. Muyldermans S. Nanobodies: Natural Single-Domain Antibodies. Annu Rev Biochem. 2013 Jun. 2; 82(1):775-97.
80. Freudl R. Signal peptides for recombinant protein secretion in bacterial expression systems. Microb Cell Fact. 2018 Mar. 29; 17(1):52.
81. González-Montalbán N, Carrió M M, Cuatrecasas S, Aris A, Villaverde A. Bacterial inclusion bodies are cytotoxic in vivo in absence of functional chaperones DnaK or GroEL. J Biotechnol. 2005 September; 118(4):406-12.
82. Chen Y J, Liu P, Nielsen A A K, Brophy J A N, Clancy K, Peterson T, et al. Characterization of 582 natural and synthetic terminators and quantification of their design constraints. Nat Methods. 2013 Jul. 2; 10(7):659-64.
83. Kulp A, Kuehn M J. Biological Functions and Biogenesis of Secreted Bacterial Outer Membrane Vesicles. Annu Rev Microbiol. 2010 Oct. 13; 64(1):163-84.
84. Bonnington K E, Kuehn M J. Protein selection and export via outer membrane vesicles. Biochimica et Biophysica Acta (BBA)—Molecular Cell Research. 2014 August; 1843(8):1612-9.
85. Fiocca R, Necchi V, Sommi P, Ricci V, Telford J, Cover T L, et al. Release of Helicobacter pylori vacuolating cytotoxin by both a specific secretion pathway and budding of outer membrane vesicles. Uptake of released toxin and vesicles by gastric epithelium. J Pathol. 1999 June; 188(2):220-6.
86. Balsalobre C, Silvan J M, Berglund S, Mizunoe Y, Uhlin B E, Wai S N. Release of the type I secreted alpha-haemolysin via outer membrane vesicles from Escherichia coli. Mol Microbiol. 2006 January; 59(1):99-112.
87. Wurm D J, Marschall L, Sagmeister P, Herwig C, Spadiut O. Simple monitoring of cell leakiness and viability in Escherichia coli bioprocesses-A case study. Eng Life Sci. 2017 June; 17(6):598-604.
88. Bao R M, Yang H M, Yu C M, Zhang W F, Tang J B. An efficient protocol to enhance the extracellular production of recombinant protein from Escherichia coli by the synergistic effects of sucrose, glycine, and Triton X-100. Protein Expr Purif. 2016 October; 126:9-15.
89. Yang H, Lu X, Hu J, Chen Y, Shen W, Liu L. Boosting Secretion of Extracellular Protein by Escherichia coli via Cell Wall Perturbation. Appl Environ Microbiol. 2018 Oct. 15; 84(20).
90. Coyne M J, Roelofs K G, Comstock L E. Type VI secretion systems of human gut Bacteroidales segregate into three genetic architectures, two of which are contained on mobile genetic elements. BMC Genomics. 2016 Dec. 15; 17(1):58.
91. Wettstadt S, Filloux A. Manipulating the type VI secretion system spike to shuttle passenger proteins. PLoS One. 2020 Feb. 26; 15(2):e0228941.
92. Chatzidaki-Livanis M, Geva-Zatorsky N, Comstock L E. Bacteroides fragilis type VI secretion systems use novel effector and immunity proteins to antagonize human gut Bacteroidales species. Proceedings of the National Academy of Sciences. 2016 Mar. 29; 113(13):3627-32.
93. Engelhart M J, Glowacki R W P, Till J M, Harding C V., Martens E C, Ahern P P. The NQR Complex Regulates the Immunomodulatory Function of Bacteroides thetaiotaomicron. The Journal of Immunology. 2023 Sep. 1; 211(5):767-81.
94. Pardue E J, Sartorio M G, Jana B, Scott N E, Beatty W, Ortiz-Marquez J C, et al. Dual Membrane-spanning Anti-Sigma Factors Regulate Vesiculation in Gut Bacteroidota. bioRxiv. 2023 Jul. 13;
95. Lubkowicz D, Horvath N G, James M J, Cantarella P, Renaud L, Bergeron C G, et al. An engineered bacterial therapeutic lowers urinary oxalate in preclinical models and in silico simulations of enteric hyperoxaluria. Mol Syst Biol. 2022 Mar. 7; 18(3).
96. Kurtz C B, Millet Y A, Puurunen M K, Perreault M, Charbonneau M R, Isabella V M, et al. An engineered E. coli Nissle improves hyperammonemia and survival in mice and shows dose-dependent exposure in healthy humans. Sci Transl Med. 2019 Jan. 16; 11(475).
97. Li J, Zhang W, Wang C, Yu Q, Dai R, Pei X. Lactococcus lactis expressing food-grade β-galactosidase alleviates lactose intolerance symptoms in post-weaning Balb/c mice. Appl Microbiol Biotechnol. 2012 Dec. 8; 96(6):1499-506.
98. Cao H, Zhou T, Tang H, Feng P, Ali G, Liu P, et al. Genetically encoded probiotic EcN 1917 alleviates alcohol-induced acute liver injury and restore gut microbiota homeostasis. J Funct Foods. 2021 October; 85:104661.
99. Wegmann U, Carvalho A L, Stocks M, Carding S R. Use of genetically modified bacteria for drug delivery in humans: Revisiting the safety aspect. Sci Rep. 2017 May 23; 7(1):2294.
100. Wo J, Lv Z Y, Sun J N, Tang H, Qi N, Ye B C. Engineering probiotic-derived outer membrane vesicles as functional vaccine carriers to enhance immunity against SARS-CoV-2. iScience. 2023 January; 26(1):105772.
101. Surendran N, Barnard D, Hellman J, Michel L V, Bettinger J, Pichichero M E, et al. Dual orientation of the outer membrane lipoprotein Pal in Escherichia coli. Microbiology (NY). 2015 Jun. 1; 161(6):1251-9.
102. Dean S N, Thakur M, Spangler J R, Smith A D, Garin S P, Walper S A, et al. Different Strategies Affect Enzyme Packaging into Bacterial Outer Membrane Vesicles. Bioengineering. 2023 May 11; 10(5):583.
103. Wan X, Ho T Y H, Wang B. Engineering Prokaryote Synthetic Biology Biosensors. In: Handbook of Cell Biosensors. Cham: Springer International Publishing; 2022. p. 283-318.
104. Koch M, Pandi A, Borkowski O, Batista A C, Faulon J L. Custom-made transcriptional biosensors for metabolic engineering. Curr Opin Biotechnol. 2019 October; 59:78-84.
105. Chang H J, Mayonove P, Zavala A, de Visch A, Minard P, Cohen-Gonsaud M, et al. A Modular Receptor Platform To Expand the Sensing Repertoire of Bacteria. ACS Synth Biol. 2018 Jan. 19; 7(1):166-75.
106. Lahav-Mankovski N, Prasad P K, Oppenheimer-Low N, Raviv G, Dadosh T, Unger T, et al. Decorating bacteria with self-assembled synthetic receptors. Nat Commun. 2020 Dec. 10; 11(1):1299.
107. Guiney D G, Hasegawa P, Davis C E. Plasmid transfer from Escherichia coli to Bacteroides fragilis: differential expression of antibiotic resistance phenotypes. Proceedings of the National Academy of Sciences. 1984 November; 81(22):7203-6.

We claim:

1. A recombinant polynucleotide comprising a promoter, a ribosome binding site, a sequence encoding a secretion carrier, and a sequence encoding a heterologous protein.

2. The recombinant polynucleotide of claim 1, wherein the promoter is a Bacteroides promoter.

3. The recombinant polynucleotide of claim 2, wherein the promoter is an inducible promoter.

4. The recombinant polynucleotide of claim 2, wherein the promoter is a constitutive promoter.

5. The recombinant polynucleotide of claim 6, wherein the ribosome binding site is derived from BT1311, or is RBS8 or A21 RBS.

6. The recombinant polynucleotide of claim 1, wherein the secretion carrier is a truncated membrane-associated Bacteroides lipoprotein.

7. The recombinant polynucleotide of claim 1, wherein the secretion carrier is a full-length membrane-associated Bacteroides lipoprotein.

8. The recombinant polynucleotide of claim 1, wherein the recombinant polynucleotide encodes a secretion carrier comprising a positively charged region of about 3 to 9 amino acids, a hydrophobic region of about 13-34 amino acids, and a lipoprotein secretion sequence.

9. The recombinant polynucleotide of claim 1, wherein the charged region comprises a polypeptide as set forth in SEQ ID NO:123 or SEQ ID NO:124, and the lipoprotein secretion sequence comprises a polypeptide as set forth in SEQ ID NO:125.

10. The recombinant polynucleotide of claim 1, wherein the heterologous protein is a therapeutic protein that is an antibody or specific binding fragment thereof, a cytokine, or a growth factor.

11. The recombinant polynucleotide of claim 10, wherein the antibody or specific binding fragment thereof is a scFv, Fab, Fab′, Fv, F(ab′)₂, a minibody, a diabody, a triabody, a tetrabody, a tandem di-scFv, a tandem tri-scFv, an immunoglobulin single variable domain (ISV), a V_{HH}, a humanized V_{HH}, a camelized V_{H}, a single domain antibody, a domain antibody, or a dAb.

12. The recombinant polynucleotide of claim 1, further comprising a linker or cleavage site positioned between or within the secretion carrier and the heterologous protein.

13. A recombinant polypeptide comprising:
(i) a secretion carrier comprising a positively charged region of about 3 to about 9 amino acids, a hydrophobic region of about 13 to 34 amino acids, and a lipoprotein export sequence; and
(ii) a heterologous polypeptide.

14. The recombinant polypeptide of claim 13, wherein the secretion carrier is a truncated membrane-associated Bacteroides lipoprotein.

15. The recombinant polypeptide of claim 13, wherein the secretion carrier is a full-length membrane-associated Bacteroides lipoprotein.

16. The recombinant polypeptide of claim 13, wherein the heterologous protein is a therapeutic protein comprising an antibody or specific binding fragment thereof, a cytokine, or a growth factor.

17. The recombinant polypeptide of claim 13, wherein the positively charged region is set forth in SEQ ID NO:123 or SEQ ID NO:124, and the lipoprotein secretion sequence is set forth in SEQ ID NO:125.

18. The recombinant polypeptide of claim 16, wherein the antibody or specific binding fragment thereof is a scFv, Fab, Fab′, Fv, F(ab′)₂, a minibody, a diabody, a triabody, a tetrabody, a tandem di-scFv, a tandem tri-scFv, an immunoglobulin single variable domain (ISV), a V_{HH}, a humanized V_{HH}, a camelized V_{H}, a single domain antibody, a domain antibody, or a dAb.

19. A vector comprising the polynucleotide of claim 1.

20. A recombinant cell comprising the polynucleotide of claim 1.

21. The recombinant cell of claim 20, wherein the cell is a Bacteroides cell.

22. The recombinant cell of claim 21, wherein the Bacteroides cell is a B. thetaiotaomicron, B. ovatus, B. fragilis, B. vulgatus, B. distasonis or B. uniformis cell.

23. A method of exporting a heterologous polypeptide from a cell comprising delivering the recombinant polynucleotide of claim 1 to the cell.

24. The method of claim 23, wherein the heterologous polypeptide is freely soluble in an extracellular space of the cell; bound to an external surface of an outer membrane vesicle (OMV); or held within an OMV lumen.

25. A method of treatment comprising administering the recombinant cell of claim 19 to a subject.

26. The method of claim 25, wherein the subject has an intestinal disorder.

27. The method of claim 26, wherein the intestinal disorder is inflammatory bowel disease (IBD) or Crohn's disease.

28. The method of claim 25, wherein the recombinant cell is administered orally or intrarectally.


SEQUENCE LISTING (235 sequences)

SEQ ID NO: 1 - protein, 351 aa, synthetic construct
MKKNLLLLLCLFCLVNVCHAKAKDTFTVFQLNLWHGCTKVPNGDQGIIDVLDQMDADVVFLCEIRDGKQFIPHVIEELEKRGKHYYGETFDLAIGVLSKFKPDSWTKCCIVPGDEGRAMVKMVATIEGQPVSFYSCHLDYRHYQCYMPRGYNGTTWKKMDKPITDEEEVLKANRQSFRDETIRAFIQEVQSDIQQGRPIIMGGDFNEPSHLDWQADTKDLWDHNGAIIHWDCSMMLSKAGFKDAYREKFPNTVRYPGFTFPAGNKLAEEAKLEKLAWAPEADERDRIDFIYYYPLESMLSMKDSKLVGPSETVVRGKITENDSKDKILTPSCIWPSDHKGNLATFKIRKKR

SEQ ID NO: 2 - protein, 47 aa, synthetic construct
MRNLKWLYACSLAIAFGVLSFVTVSCHDDDDEPKQEPGEVIETPAPV

SEQ ID NO: 3 - protein, 39 aa, synthetic construct
MMKKTILLTSIIAIAIVSMLSSCDSEKDLYDPSYQTANP

SEQ ID NO: 4 - protein, 236 aa, synthetic construct
MYMKKKRLGQWMLLAIVCLSFSVGETYAQKHEFANYKRYATENAALAQPVKKEKRVVFMGNSITEGWVRTHPDFFKTNGYIGRGISGQTSYQFLLRFREDVINLSPALVVINAGTNDVAENTGAYNEDYTFGNIASMAELAKANKIKVILTSVLPAAEFPWRREIKDAPQKIQSLNARIEAYAKANKIPFVNYYQPMVVGENKALNPQYTKDGVHPTGEGYDIMEALIKQAIEKAL

SEQ ID NO: 5 - protein, 293 aa, synthetic construct
MKLRIYTLLIAFCAAWSLHSCDNDDDESIAVPTPLQEAFSTRYPNVKNVKWETKAGYYVADFYDGYEASAWFTTDGNWHMTETDIPYTALPDPVKSAFEASDYKTWKRDDVDKLERQGIETVYVIEVENQNQEVDLYYSADGVLIKSIADTDDHENEHLPTTQLPAVMKTFIDGKYAGARIVEVDVEDDKNDWDFGFTEVDIIHFDSGLNRNVSKEVLFDKGREWYSTSWEVRRNELPTAVTNAISTEYAGYQMDDAEYFEMATETSYYQIELEGNNSPDIDIKVTADGTILK

SEQ ID NO: 6 - protein, 145 aa, synthetic construct
MKKLVFLLVCLFTLQTVARADDDKPIQVTQMPQLAQQFIKQHFSDSKVALAKMESDFLYKSYEVIFTNGNKVEFDKKGNWEEVDCKHTSVPVAIIPAAIQKYVTTNYPDAKVLKIERDKKDYEVKLSNRTELKFDLKFNLIDIDN

SEQ ID NO: 7 - protein, 199 aa, synthetic construct
MKKNILFIAWVALFSLFASNSQAQSLKDLLNKDNISKVVNAITGTPETIDMTGTWTYSGSAVEFESDNLLMKAGGAAAATMAENKLNEQLSKVGIKEGQMSFTFNADSTFTSTVGKKKLSGTYSYNASTKQVDLKYLKLLNLHAKVNCTSNSMDLLFNSDKLLKLMTFLGSKTNSTALKTVSSLAENYDGMMLGFGLKK

SEQ ID NO: 8 - protein, 43 aa, synthetic construct
MMKKIKILSLLVCLIALFTACHDEEDKTNYGSRTVLVYIAGDN

SEQ ID NO: 9 - protein, 568 aa, synthetic construct
MKKLLILPLALFLLVQSGLAQTPKWVEKAKRAVFSVITYDKNDKMLNTGNGFFVSEDGLALSDYSLFKGAERAVIVTAEGKQMPVSLILGADDMYDVIKFRVAITEKKVPSLVVATTAPAAGADAWMLPYSTQKSIACVSGKVKDVSKIAGEYHYYTLSMQMKDKMVSCPVMNAEGQVFGISQKSSGVDTVTTCYAAGAAFAMSQKISALSLGDVALKNIGIRKGLPEAEDQALVYLFMASTQMSADEYEKLLDDFIRQFPSSTDGYIRRANYYVAKGKDDQSYFDKAVADFNQALKVAAKKDDVYYNIAKLIYGYQLSKPETTYKDWTYDTALKNLRQAMAIDPLPVYTQLEGDILFAQQDYAGALAAYEKVNASNLASAASFFSAAKTKELLKADAKEVLALMDSCIARCPQPVTANFAPYLLERAQIYMNNDQARNAMLDYDAYYKAVNGQVNDLFYYYREQAALKARQYQRALDDIVKAVELSPKDLTYRAEHAVVNLRVGRYEESMKILNEILKDEPKYGEAYRLLGLCQIQLKKTDEACGNFNKAKELGDPNVDELIKKYCK

SEQ ID NO: 10 - protein, 47 aa, synthetic construct
MNKKFLSVILFGALMTVSTGTFVSCKDYDDDINNLQEQIDGQKNDLN

SEQ ID NO: 11 - protein, 42 aa, synthetic construct
MKTKTLSYTPILFCILILWSACKDDADAPLRFYDSKYEVPMG

SEQ ID NO: 12 - protein, 41 aa, synthetic construct
MKKNFLMWSFAMALLTGTLCTSCDETEGAVAPEETQSVQKG

SEQ ID NO: 13 - protein, 45 aa, synthetic construct
MFYCMKKNLLFAAMAVTALASCSNDDVVDVNNGGGISFRASLDRA

SEQ ID NO: 14 - protein, 37 aa, synthetic construct
MRKVFILLFISLALLSCQKEENNRKTEYQVLTSKDAS

SEQ ID NO: 15 - protein, 42 aa, synthetic construct
MKKFFYLSALSLGMMCSITACSDDDTTTIDAKNLDYTAENAS

SEQ ID NO: 16 - protein, 45 aa, synthetic construct
MIFHRIFRFTYFLIFCIWGLQSCTKDDLIETPADTDSETTENPYG

SEQ ID NO: 17 - protein, 41 aa, synthetic construct
MRKEKLYTGCLLLMALITGSCSEEENPEVRPATKPAEPYTS

SEQ ID NO: 18 - protein, 41 aa, synthetic construct
MNYFKSIMLTAFALFALAACDTDDLRDDVDNLKDRVESLEA

SEQ ID NO: 19 - protein, 60 aa, synthetic construct
MELLKSHFFKMRKAYIESFIILLFLSCCPFIVSSCHEEEKEEIPESPFDEEDIQHEQDLN

SEQ ID NO: 20 - protein, 56 aa, synthetic construct
MENLKVFFTRRPERLLGRCGMLFLMAFFVMNLSSCNDDFIEDDGGEVIPPEDIVIP

SEQ ID NO: 21 - protein, 485 aa, synthetic construct
MTTSRLYASKKFMGALLLMMGFVLPANAQFLRTSYFMEGTHYRMQLNPALAPTSGYFNIPAIGSLNVSASSNSLGTQDIIDIIDNSDGFYNNQKFMNRLSNDNRLNVNVNTDILSFGWYKGKNFWSVNVGARVDLGAQIPKSMFSFLHDIDQDGFSWNNSKFDIGKEELNINAYTEVGIGYARAINDRLSVGGKFKVLLGMGNLNLKVDGMNVDANLPLNINDITDVNQIRDYHAKMKVNARLESSFKGMDLVENTSDPDPRKHYIDDFDFNGFGIAGYGGAIDLGASYKILDNLTVSASVLDLGFIKWSKGSTSVATAKGSMDYDGKDYALTPEGLEDFQRDAQDFMNRVEGGDVLNYEMLQLQKEDVVESRTTSLHSTVVIGAEYELLDKWLAIGALSTTRFTKPKTQTEITLSANIRPKSWLNAAISYSMIQSAGKSFGLAVKLGPLFIGTDYMFFGKSTKTVNGFVGISVPLGGKKSSKEG

SEQ ID NO: 22 - protein, 39 aa, synthetic construct
MMKKGILFVLTAAFLASCQQEENEGVASVDRVTITPIIT

SEQ ID NO: 23 - protein, 48 aa, synthetic construct
MNYSCRKTIVPIIIGTLLSGACSNDEPTGGKGHQQTYSVVLKGITVAG

SEQ ID NO: 24 - protein, 42 aa, synthetic construct
MKMLRIIMILLGALLLTNCSGDFEQETGIVPSHSGQVSFLFG

SEQ ID NO: 25 - protein, 53 aa, synthetic construct
MKIPQIRCSDVNAVKRIIFLLVLLIPFCLAGCSSDDDEGDNGGEGPNEDAPVS

SEQ ID NO: 26 - protein, 44 aa, synthetic construct
MKTFRFIYQFVLLLMCTTACNEYDDSELWDRVNSLDDRVTSIEN

SEQ ID NO: 27 - protein, 41 aa, synthetic construct
MRKYLYLSAVCVCMALCFVGCSKDDDEPGGKGAMYEVTIEQ

SEQ ID NO: 28 - DNA, 1053 bp, synthetic construct
  source 1..1053: mol_type = other DNA
atgaaaaagaacttactattattactttgcttgttttgtctggtcaatgtttgccatgcaaaagcgaaagacacttttaccgttttccaattaaacctgtggcatggatgtactaaagtacccaacggagatcaaggcattattgatgtactggatcagatggatgcggatgtcgtttttctgtgcgaaatcagagacggaaaacagtttatccctcatgtgatcgaagagctggagaagagaggaaaacattactatggtgaaacgtttgatcttgcaatcggtgttttgagtaaattcaagccggacagttggaccaaatgctgcatcgtacctggtgacgaaggtcgtgctatggtgaaaatggttgcaacaatcgaaggccaacccgtatctttctattcttgtcatttggactaccggcattatcaatgctatatgccacgtggatataatggtactacctggaagaaaatggataagccgataacggatgaagaagaagtcttgaaagcaaacagacagtctttccgggatgaaaccatccgtgcttttattcaggaagtacaatctgacattcaacaaggccgcccgataatcatgggaggagattttaatgagccctctcatttagactggcaggcagatacaaaggatttgtgggatcataatggggctatcattcattgggactgctcaatgatgctgagcaaagcaggattcaaagatgcttatcgtgaaaaatttcccaatacagttcgatatcccggttttacattccctgccggaaacaaactcgcagaagaggctaaactggaaaagttagcttgggcgcctgaggcagacgaacgtgaccggattgatttcatttattattatccattggagtcgatgctttccatgaaggatagcaagttggtaggtccctccgaaacagttgtccggggaaaaataacagaaaacgattctaaggataagattctgacgccttcttgtatttggcccagcgaccataaagggaatcttgctacatttaagattcggaagaaacgg

SEQ ID NO: 29 - DNA, 141 bp, synthetic construct
  source 1..141: mol_type = other DNA
atgagaaatttaaaatggttgtatgcatgctctttggcgatagccttcggtgtgttgtcttttgtaacagtttcctgtcatgatgatgacgatgaaccaaagcaagaaccaggtgaagtgattgaaactcccgctccggtt

SEQ ID NO: 30 - DNA, 117 bp, synthetic construct
  source 1..117: mol_type = other DNA
atgatgaaaaaaacaattctgcttacatctattattgcgatagcaatagtaagcatgttatccagttgcgtagattccgagaaggatctatatgatccgtcttatcagacagccaat

SEQ ID NO: 31 - DNA, 708 bp, synthetic construct
  source 1..708: mol_type = other DNA
atgtatatgaaaaagaaaagactgggtcaatggatgttgttggccatcgtttgtttgagtttctcagtaggagagacctatgcacagaaacatgagttcgctaattacaagcgttatgcgacagagaatgcagcacttgcacaacctgtaaagaaggaaaaaagagtggtctttatgggcaattccattacggaaggatgggtgaggacacatcctgatttcttcaaaacaaatggatatatcggtcgtggtatcagtggccagacgtcctatcagtttttacttcgttttagggaagatgtgatcaatctttctccggcattggttgtcattaatgcaggaacaaatgatgtagccgaaaatacaggtgcttataatgaagattatacattcggaaatattgcatctatggcagaactggcgaaggcaaataaaatcaaagtgattctgacttcagtattgccggctgctgaatttccgtggagacgggagattaaagatgctcctcaaaagattcagtcattgaatgcccgcatagaagcttacgccaaagctaataaaattccgtttgtgaattattatcagccaatggtggtgggcgagaataaggccttgaatcctcaatatacgaaagatggggtacatcctacaggagaaggatatgatatcatggaagcactcattaaacaggctattgaaaaagcgctt

SEQ ID NO: 32 - DNA, 879 bp, synthetic construct
  source 1..879: mol_type = other DNA
atgaaactcagaatttatacactactgatagcattctgtgcagcatggagcctgcacagctgcgacaacgatgacgatgaatcaattgctgttcctaccccattgcaagaagcattctcgaccaggtatcctaacgtcaagaacgtaaagtgggaaacaaaggcaggatattatgtagcggacttctatgatggatatgaggcatccgcatggttcacaacggatggcaactggcatatgactgaaacggatatcccgtacaccgcattgcccgatccggtaaagagtgcattcgaagcaagtgactataagacatggaaaagagacgatgtggataaactcgagcggcaaggcatcgaaacagtctacgtcattgaggtggaaaaccaaaatcaggaagtcgatctgtattattctgccgacggagtgctgattaaaagcattgcagacacagacgatcatgaaaacgagcaccttccgaccactcaactgcccgccgtcatgaagactttcattgatgggaaatatgcaggagcaagaatcgtggaagtagacgtggaagatgataaaaacgactgggatttcggatttacggaagtagatatcatccatttcgacagtggcctgaaccgcaacgtttccaaagaagtcctgtttgataaaggcagagagtggtattctacctcatgggaggtacggagaaacgaactgcccacagcagtgaccaatgccatcagcaccgagtatgcaggatatcagatggatgatgccgaatactttgagatggcaacagaaacatcctattatcagattgaactggaaggaaataattctccggatatagatattaaagtaaccgcagacggtacgattctcaaa

SEQ ID NO: 33 - DNA, 435 bp, synthetic construct
  source 1..435: mol_type = other DNA
atgaaaaagttagtattcttattagtatgcttgttcactttacagacagtagcacgtgcagatgacgataaacctatccaggtgactcaaatgccacaattagcacaacagttcatcaaacagcatttctctgacagcaaagtggcactggcaaaaatggaaagtgacttcctttacaaaagctatgaagtgattttcacgaatggtaataaagtggaatttgacaagaagggaaactgggaggaagtagattgcaagcacacctccgtacctgttgccattattcctgccgccattcagaaatatgtaacaactaattatccggatgcaaaagtgctgaagatagaacgtgataagaaagattacgaagtaaagctatccaaccgtacagaactgaaatttgacctgaagtttaatctgattgatattgataat

SEQ ID NO: 34 - DNA, 597 bp, synthetic construct
  source 1..597: mol_type = other DNA
atgaaaaagaacattttatttattgcatgggttgcattattctcactctttgcaagcaacagtcaggcacaatcattaaaggatctgctcaacaaagacaacatctccaaagtagtaaatgccatcaccgggacacccgaaacgatagacatgacaggtacatggacttacagcggttcggcagtagaatttgaatcggacaatctgctgatgaaagcaggaggagccgcagccgccacgatggcagaaaacaaattgaacgagcaattgagtaaagtgggcatcaaagaaggacaaatgagcttcacgttcaacgccgacagtactttcaccagcaccgtcggaaaaaagaaactgagcggcacttattcgtacaatgcttccaccaagcaggtagacttaaagtacttgaaactgctgaatctgcatgcgaaagtgaactgcacttccaactctatggatttattgttcaactccgacaagctgcttaaattaatgacattcctcggcagcaagacaaacagcacagcactgaaaactgtcagctcactggctgaaaactacgacgggatgatgctgggattcgggttgaagaaa

SEQ ID NO: 35 - DNA, 129 bp, synthetic construct
  source 1..129: mol_type = other DNA
atgatgaaaaagattaaaatattatcccttttggtgtgtttgattgctctatttacggcttgtcatgatgaagaggataaaactaattatggttcgcgtactgtgttagtttatattgcgggtgacaac

SEQ ID NO: 36 - DNA, 1704 bp, synthetic construct
  source 1..1704: mol_type = other DNA
atgaagaaacttttaatcttacctttagcgcttttcctcttggtgcagagcggtctggcgcaaaccccgaaatgggtggagaaggcgaaacgtgctgttttctcagtaataacttatgataagaatgacaagatgctgaataccggaaacggatttttcgtatccgaagacggactggctctttctgactacagtcttttcaaaggggctgagcgtgcggtgattgtcacagccgaaggcaagcaaatgccggtcagcctgattttgggagcagatgatatgtacgatgtgatcaagtttcgtgtggccatcaccgagaagaaagtgccgtcactggtagtggcgactactgctccggctgcaggcgctgacgcatggatgcttccttattctacgcagaagagcattgcctgcgtttcgggtaaagtgaaggatgtatcgaagattgccggagaataccattactataccttgagtatgcagatgaaagacaagatggtgagttgtccggtgatgaatgcggaaggccaggtgttcggtatttcacagaagtcctccggtgtggatacggtaacgacttgttatgcggcaggggcagcttttgcaatgtcgcagaagattagtgctctgtctttgggagacgttgctctgaagaatatcggtatccggaaaggattgcccgaagcggaggatcaggcattggtgtatctgtttatggcttccacgcagatgtcggctgatgaatatgagaaattgctggacgatttcattcgccagttccccagcagtaccgatggatatatccgccgtgccaactattacgtagcaaaaggtaaggatgaccagtcttattttgacaaggctgtagccgactttaatcaagccttgaaggtggcggctaaaaaggatgatgtgtattataatatcgccaagctgatatacggttatcagctttcaaaaccggaaacgacttataaagactggacgtatgatacggctttgaaaaatctccgtcaggcgatggctatcgatcctttgcccgtttatacgcagttggaaggagatatactttttgcacagcaggactatgcaggcgctttggccgcttatgagaaggtgaatgccagcaatctggcttcagcagcctcattctttagtgccgccaagacgaaagaattgctgaaggcagatgccaaagaagtgttggcattgatggacagttgcattgctcgttgtccgcagcctgtcacggcaaattttgctccttatctgctggaacgtgcacagatatatatgaacaacgatcaggcaagaaatgcaatgctcgattatgatgcttattataaggctgtgaacggacaggtcaatgaccttttctattattatcgtgagcaggctgcgctgaaagcccgtcaatatcagcgtgcattggatgatatcgtgaaagcggttgaattgagccccaaagatttgacttatcgggcagaacatgcagtggttaatctgcgtgtaggacgttacgaagaatccatgaagatactgaatgaaattctgaaagacgagccgaaatatggggaagcataccggttgttgggcctttgtcagatacagttgaagaagacggacgaagcttgcggaaacttcaataaagcgaaggaactgggtgatccgaatgtggatgaacttataaaaaaatattgtaaa

SEQ ID NO: 37 - DNA, 141 bp, synthetic construct
  source 1..141: mol_type = other DNA
atgaacaaaaagtttttaagtgtgatcctgtttggagccttgatgacggtttctacgggcacttttgtctcttgtaaggattatgatgatgacattaataatctacaggagcaaattgatggtcagaagaatgatctgaac

SEQ ID NO: 38 - DNA, 126 bp, synthetic construct
  source 1..126: mol_type = other DNA
atgaaaactaaaaccctctcttacacccccatcttgttttgcattcttattttatggagtgcctgtaaagacgatgccgatgcccccctgcggttttacgacagcaaatacgaagtgcccatggga

SEQ ID NO: 39 - DNA, 123 bp, synthetic construct
  source 1..123: mol_type = other DNA
atgaagaagaattttttgatgtggagctttgcaatggctctgttgacaggtacgctttgtacctcttgtgatgaaactgagggagccgttgctccggaagaaacgcaatccgtacagaaggga

SEQ ID NO: 40 - DNA, 135 bp, synthetic construct
  source 1..135: mol_type = other DNA
atgttttactgtatgaaaaagaatcttttattcgcagctatggctgtaacagcgttggcctcttgttccaacgatgatgtcgttgacgtgaacaatggcggtggcatttccttccgtgcttctttggacagggcc

SEQ ID NO: 41 - DNA, 111 bp, synthetic construct
  source 1..111: mol_type = other DNA
atgagaaaggtatttattcttttgtttatttctttagcattgttatcctgtcaaaaagaggaaaacaatagaaaaacagagtatcaagtattaacaagtaaggacgcatcc

SEQ ID NO: 42 - DNA, 126 bp, synthetic construct
  source 1..126: mol_type = other DNA
atgaaaaagtttttttatttgtcggctctttcattgggcatgatgtgttccattacggcttgtagtgatgatgacactacgactatagacgctaaaaatcttgactatacggcggagaatgcaagt

SEQ ID NO: 43 - DNA, 135 bp, synthetic construct
  source 1..135: mol_type = other DNA
atgatattccacagaatctttcgtttcacctattttcttattttctgtatatggggactgcaaagctgcaccaaagacgacttaatagaaacgccggcagacacagacagcgaaacaacagagaacccgtatgga

SEQ ID NO: 44 - DNA, 123 bp, synthetic construct
  source 1..123: mol_type = other DNA
atgagaaaagaaaaattatataccggatgtctgttactaatggcattaatcacaggtagttgctcggaagaagaaaacccggaagtccgcccggctacaaaacccgcagagccatatacaagc

SEQ ID NO: 45 - DNA, 123 bp, synthetic construct
  source 1..123: mol_type = other DNA
atgaattactttaaatcaatcatgttaaccgcatttgcgctgtttgcattggcagcgtgcgatacagacgacttgagagacgatgttgacaatctgaaagaccgcgtagagtctttggaagca

SEQ ID NO: 46 - DNA, 180 bp, synthetic construct
  source 1..180: mol_type = other DNA
atggagctattgaaaagtcatttttttaaaatgagaaaggcttatattgagtcgtttattattcttttgtttctgtcctgttgtcctttcattgtttcttcctgtcacgaggaagagaaagaggagatacctgaatctccttttgacgaagaagatattcaacacgaacaagatctgaat

SEQ ID NO: 47 - DNA, 168 bp, synthetic construct
  source 1..168: mol_type = other DNA
atggaaaatttgaaagttttctttacgagaaggcctgaacgcctgctagggcgatgtggcatgttgtttttaatggcattttttgtgatgaacttgtcttcctgtaatgatgactttattgaagatgatgggggagaagttattcctcctgaggatattgttatcccg

SEQ ID NO: 48 - DNA, 1455 bp, synthetic construct
  source 1..1455: mol_type = other DNA
atgacaacaagcagactttatgcatctaagaagttcatgggggctttacttcttatgatgggttttgttttacctgccaatgcgcagtttttacgtacttcatattttatggaaggtactcattatcgtatgcagttgaatccggctttggcaccgactagcggatattttaatattccggcaattggttcgttgaatgtatcggcaagttcaaattctttgggtacacaagatattatagatattattgataatagtgatggtttctataataatcagaaattcatgaatcgtttgtctaatgataaccggttgaatgtcaatgtcaatacggatattctctctttcggatggtataaaggaaagaacttttggtcggttaatgtaggtgcccgtgtagatttaggtgctcagatacctaagtccatgtttagcttcttgcacgatattgatcaagatggtttcagttggaataattcaaaatttgatattggaaaggaagaattgaatataaatgcttatactgaagttggtattggttatgcccgcgctattaatgatcgcttgtcagtaggtggtaaatttaaggtattacttggtatgggtaacttgaatttgaaggtagacggaatgaatgtagatgcgaatttgccactaaacattaacgatattactgatgtcaatcagattcgtgattatcatgctaaaatgaaagtaaatgcgcgtcttgagagctctttcaaaggaatggatttggtggaaaatacgagtgatcctgatcctcggaaacactatattgatgattttgattttaacggatttggcattgcaggttatggtggtgccatcgatttaggggcatcttataagattcttgataatttgacagtgtctgcttctgtgttggatttgggttttatcaagtggtcaaaggggagtacaagtgttgcaacagctaaaggaagtatggattatgatggcaaagactatgcacttactccggaagggttagaggatttccagcgggatgcccaagattttatgaatcgtgtggaaggtggggatgttttgaattacgaaatgctccaactgcaaaaagaagatgttgttgagtctcgtaccacttctcttcattctacagtagtaattggtgctgagtatgaactgttggataaatggttggcgattggtgctctttccactacccgcttcactaagccgaaaactcagactgaaattacactttcggctaatattcgtccgaaaagttggttgaatgcggcaatcagttattctatgattcagagtgctggtaaatcatttggtttggctgtgaaacttgggcctttgtttatcggtacggactatatgttctttggaaaaagtacaaagacagtaaatggatttgttggtatttctgtgccattgggcggaaagaaatccagtaaagaagga

SEQ ID NO: 49 - DNA, 117 bp, synthetic construct
  source 1..117: mol_type = other DNA
atgatgaaaaaggggatattatttgtgctgacagcagcatttctggcatcttgccagcaggaggaaaacgagggagtagcatctgtggaccgggtgaccatcactcctattatcaca

SEQ ID NO: 50 - DNA, 144 bp, synthetic construct
  source 1..144: mol_type = other DNA
atgaattattcttgcagaaaaaccatcgttcccattataataggaacattactttcaggcgcctgctcgaacgatgaaccaaccgggggaaaaggccatcaacaaacttattcagtggtattaaaaggaattacggtagcagga

SEQ ID NO: 51 - DNA, 126 bp, synthetic construct
  source 1..126: mol_type = other DNA
atgaagatgctaagaatcataatgatattactgggcgcattattgcttaccaactgttcgggcgattttgaacaggagaccggcattgttccttcccattccggacaagtgtcgtttttgtttggg

SEQ ID NO: 52 - DNA, 159 bp, synthetic construct
  source 1..159: mol_type = other DNA
atgaagatacctcaaataagatgtagcgatgtgaatgcagtgaagagaatcatttttcttttggtgctgctaattcccttttgtctggcaggatgttcgtccgacgatgatgaaggagataatggaggagaaggtccgaacgaagatgctccggtttct

SEQ ID NO: 53 - DNA, 132 bp, synthetic construct
  source 1..132: mol_type = other DNA
atgaaaacatttagatttatttatcagtttgtgctgttattaatgtgcacaactgcgtgtaacgaatacgatgacagcgaattatgggatagagttaattctttagatgacagagtgacctctattgagaac

SEQ ID NO: 54 - DNA, 123 bp, synthetic construct
  source 1..123: mol_type = other DNA
atgagaaagtatttgtatttgtcagccgtatgcgtgtgcatggcattgtgtttcgtcggttgcagcaaggacgacgacgaaccgggaggaaaaggggcgatgtatgaggtaacgatcgagcag

SEQ ID NO: 55 - protein, 197 aa, synthetic construct
MIKYIMRKIVLSLFALCCFSAVMAQQKTRSLSVELLGAQTIVGINYDSRFDVNSGLGYRVGIGYGYGDNSGWPDQKINGVGVPLELNYLLGKKNSKLELGFGASLGVYQVKETIGYVKDTGWYPGGENTDETSPKTEFYTSSKTQFGYFFFGDIGYRYQRPNGFMFRVGVSPSFNFGDKYGLNKAAFFPYIGFGWSF

SEQ ID NO: 56 - protein, 820 aa, synthetic construct
MMKKTILLTSIIAIAIVSMLSSCVDSEKDLYDPSYQTANPMGDGFAAPDGFDWNMTTTSILNIEIDDELYNQIEILDANPFSTSDYHILAKGVAKKGQAFSQEINYTEGTNYLYIRKTDSRSRVSISTWDVSKNKEFVGSRTTRVAKATIGSYNIPEKYPEETYDTTGAIELTGNTNWNQSNHHLEAGKSYIIKNKFNGEINHTSGYLNGGRFTIFVEGEWTPSQNQIQSADIIILKGGKINTDSFTSFLIADNSILTIQSGGSLIGNNINLAAIGVLLKNFGTISVNSMKDLNTTSILYNAPKATINVTGKSVASWEQSVFTKGAIYNFGELTIQEGALKFNSQDATCYFYNGTEATINTPTFIIGGIGVNDGTVNAQKISNDNGGNPTFTNNCSLYAQNSFEFGGTSGTIIMNKGILAGGVENGTFIAIPSFKCGNSGSTFELNNGSMIKAEIMDIPNVTFKAAGTRSLIKSTKSISTGWTTKFNGNLDIECPEGEFAKGVPANNPNYIMENSVELYIPNGSKTIITSCGELSEIPDPTPDPEDPEFPIEVEDNKDYTYLFEDQWPLYGDYDMNDIVLTIQKRKIYTNKENKVTKFELSIDLSAAGATKSIGAAIMLDNVPANAITQSVEFSDNTLAKNFNLNNNNIESGQDYAVIPLFDDAHKVLGRDRYEQINTVSDYAGNTKPKNISFSITFNNPTISADAFNVNNLNVFIIVDGNRNPRKEIHVAGYQPTKLANTDLFGGNNDNSHHGSKKYYISKENLAWGIMVPSNFKWPLEYVNIKTAYSQFSDWVTSGGTENEKWWNDFDVNKVFQTNKN

SEQ ID NO: 57 - protein, 407 aa, synthetic construct
MRNKTLRWKTIALCSIMFCLPKTPLAGQESFGFIHRLGMEARPQYVFPTNPFLQGENERWKPIQTSFAAHLKYSFKFRPNTCADRIYGGAYQGIGVSLTTFGDKKQLGDPFSFYVFQGARIARFSPRASLNYEWNFGLSAGWKPYDNYYNSYNGAVGSRMNAYINAGVYINWAFSRYFDLIVGGDFTHFSNGNTKFPNAGINTAGAKIGLVYNFNRTEEDLSKSLYQPVTTRFPRHISYDVVLFGSWRRKGVWVGEKQIASPNAYPVAGFNFAPMYNLGYKFRVGASLDGVYDGSANVYREDVIVEYGSSSSKREFLKPGIQHQLALGLSGRAEYVMPYFTIGVGMGANVLSRGDMRGFYQILALKIAVTRSSFLHIGYNLQNFQTPNYLMLGLGFRFHNKYPKVRH

SEQ ID NO: 58 - protein, 245 aa, synthetic construct
MNILRNIILFGWMAIACTAFAQDRNADKVERPNTKKINFGIKAGFNSSMFMVSELKIKDVTIDEVQNNYKIGYFGAIFMRFNIKKHFIQPEASYNVSKGEITFDKLGSQHPAIEPDYASVQSVLHSIDFPILYGYNVVKKGPYGMSIFAGPKLRYLWGKQNEITFTNFDQKGIHEKLYPLNVSVVIGVGVNISRIFFDFRYEQGIGNISKSIVYDNINSDGSTGVSPIIFRRRDSALSFSFGFIL

SEQ ID NO: 59 - protein, 267 aa, synthetic construct
MMLRFSVITYTFGRLTTKKHNKVMKKLIFLFAFCLTVTNIFAQTDPSQLKKEGSDAFNAKNYPVAYAKFSEYLKQTNNQDSAIAYYCGMAADEVKKYAEAVTFFDIAIQKKFNIGNAYARKALALDAMKKTDEYVATLEEGLKVDPDNKTMIKNYGLHYLKAGIAAQKAGKVEEAEECFKKVIPLDHKTYKTNALYSLGVLCYNDGANILKKAAPLANSDADKYAAEKEKADGRFKEAIGYLEEATKVSPEDTKSKTMLSQVQAAMK

SEQ ID NO: 60 - protein, 263 aa, synthetic construct
MRNSKVTSMLVVLMFLLVTGIQAQTVTPSKKYITKELNNVSNFSSISVLGSPDVEYRQSNGSKTTVSIYGSDNLVDLLEVSTVNGVLQVNIKKGVKILSGERRLKVIASSPSLNQVNIKGSADVYLKGAIKGNDLNLNIAGSGDIEAENLQYTNIFALVKGSGDIDLKNVKATTVMSEVNGSGDINIKGSAQKATLTVNGSGDISAEKLAATNVVATVAGSGDIVCYASRQLDARVSGSGDIEYKGSPSVVNKQGKKNSITGK

SEQ ID NO: 61 - protein, 131 aa, synthetic construct
MKKILFLMTLLVVGVSFAFAQTNADIKFDKTTHDFGKFSENSPVVSCVFTFTNIGDAPLVIHQAVASCGCTVPEYTKEPIQPGKKGTIKVTYNGTGKYPGHFKKSITLRTNAKTEMVRLYIEGDMTPKDEK

SEQ ID NO: 62 - protein, 384 aa, synthetic construct
MSRRRFLKMAALTGAAMCVGPTLNKVTAAERVWMGKQQVANNIPAGMAAVRTRRTLGHGNTAFTVSAMGYGCMGLNHNRSQYPSREKEIALVHEAVERGVTLFDTAESYGYHINEKLVGEALKGYTDRVFVSSKFGHKFVNGVQIKTEEDSTPANIRRVCENSLRNLGVETLGMFYQHRIDPNTPIEAVAETCSKLIKEGKILHWGMCEVNVDTIRRAHKICPVTAIQSEYHLMHRMVETNGVLELCEDLGIGFVPYSPLNRGFLGGMINEYTKFDVTNDNRQTLPRFQPEAIRANYRIVEVLNAFGRTRGITPAQIALAWLMNKKPFIVPIPGTTKLSHLEENLRACDIRFTAEEIEELETAVAAIPVVGSRYDALQESKIPK

SEQ ID NO: 63 - protein, 270 aa, synthetic construct
MKVRRNLLIALSLLSLSANAQRIKGSDTVLPVAQQTAERFMNLQPDARVTVTGGGTGVGISALMDNTTDIAMASRPIKFSEKMKTKEAGQEVDEVIVAYDALAVVVHPSNPVKQLTRQQLEDIFRGKITNWKQVGGDDRKIVVYSRETSSGTYEFFKENVLKNKNYMAGSLSMPATGAIIQSVSQTKGAIGYVGLAYVSPRTKTLSVSYDGSHYAAPTVENATNKTYPIVRPLYYYYNVKNKEQVAPLIDFILSPEGQEIIKKSGYIPVK

SEQ ID NO: 64 - protein, 33 aa, synthetic construct
MAIAATLLASCNKDEEETEIQGFKVLEYRPAPG

SEQ ID NO: 65 - protein, 130 aa, synthetic construct
MKKRVLLGMASLLLSVSLLMAQEIPAGVITAFKRGSSQELSKYMGDKVNLVLQGSSANVDKKKATAMMQEFFTENKVNAFDVNHQGKRDESSFVIGTLTTTKGKFRVNCFLKKVQTQYLIHQIRIDKINE

SEQ ID NO: 66 - protein, 272 aa, synthetic construct
MIRIILILFLSFPVALCAQTYQQLSEKAIEYIEMDSLSKAEELLLQALKLEPKNAKNAMLFSNLGLVQRRLGEYDKALESYSFALNFAPLAVPILLDRAAINLEMGNTDRAYTDYCQVLDEDKQNKEALLMRAYIYVLRRDYPAARIDYNRLLEIDPQSYSGRLGLATLEQKEGKFKEALEILNKMITATPEDATLYIARADVEREMKHEDLALVDLEEAIRLNPSSADAYLLRGNIYLTQKKKALAKMDFEKAISLGIPPADLHEQLKQCK

SEQ ID NO: 67 - protein, 32 aa, synthetic construct
MMTGLTLLSCSTENDEFKKELPPTEQPSEPTG

SEQ ID NO: 68 - protein, 304 aa, synthetic construct
MKLQINLCALALLLMIPFTAMAQSKTKAKAKKEVAIQLYSVRDILNKVDNKDGKCDAAYITLLKNLAKMGYTSVEAANYNNGKFYDRTPDQFKKDVESAGLKVLSSHCTRGLSKEELASGDFSSSLQWWDQCIADHKAAGMSYIVAPWMDVPKTLKELDTYCAYYNEIGKRCKQQGMSFGYHNHAHEFQKVEDKVMYDYMIEHTNPEYVFFQMDVYWVVRGQNSPVDYFNKYPGRFKMFHIKDHREIGQSGMVGFDAIFKNAKTAGVKHLVAEIESYSMPVEKSVEVSLDYLLDAPFVKSSYAK

SEQ ID NO: 69 - protein, 107 aa, synthetic construct
MTRSGNTRNGKYMGEDKRRYHTITNILQYNLIPDFFTIFAKYCITFKNMTMEKTFAQISELFDQFSKDASLQVEKGNKAAGTRARKASLELEKLLKRFRKESLEASK

SEQ ID NO: 70 - protein, 483 aa, synthetic construct
MFLAGAFIAVGSLYAQSSDAEWQAGVAKLKETIQTNPAQANEEAEQLIKGKNKKNVELLVAIGEVYLKAGKLPEAQEYAALAKKVNGKSALASVLEGDIAFEQKNAGLASQKYEEAIYFDPSCTEAYLRYADIYKSANAALAIEKLEQLKAQEPSNTAVDKKLAEIYYLKNDFSKAAEAYSRFAMGPTATEEDLVKYAFALFLNHDFEKSLEVANMGLKKNARHAAFNRLAMYNYTDLKRFDEAIKAADAFFTESDKADYSYLDYMYYGHLLEALKKYDEAVGQYEKAIQLDPTKTDLYKNISSAYEQKNDYKKAISAYQKYYTSLDKEHQTPDLQFQFGRLYYGAGTQTDSLTINAEERKQALMAADSVFHSIAEAAPDSYLGNFWRARANSALDPETTLGLAKPFYEEVATLLESKNDPHYNSALIECYSYLGYYYLLAIENPALKAEAMANKEKSKEYWSKILAIDSTNATAKRALDGIK

SEQ ID NO: 71 - protein, 529 aa, synthetic construct
MMMKKIKVYALLVCLLATSAAQAQSFGSAAMRKLQMAEFAISNFYVDKVDEDKLVEEAIIKMLAQLDPHSTYSDAEEVKKMNEPLQGNFEGIGVQFQMIEDTLLVVQPVSNGPSEKVGILAGDRIIAVNDSAIAGVKMSTEDIMKRLRGPKGSKVNLTIVRRGVQDPLVFTVKRDKIPILSLDASYMIQPKIGYIRINRFGATTAEEFKKAMKDLQKQGMKDLILDLQGNGGGYLNAAIDLANEFLGQKELIVYTEGRTAQRSEFFAKGNGEFRDGRLIVLVDEYTASASEIVSGAVQDWDRGIIVGRRSFGKGLVQRPIDLPDGSMIRLTIARYYTPAGRCIQKPYDSSTDYNKDLIDRFNHGELMNADSIHFPDSLKVQTKKLKRTVYGGGGIMPDYFVPIDTTLYTDYHRKLVGKGVIIKFTMKFIEDHRKELADKYKKFDSFNEKFVIDDDMLATLREMGEKEGVKFNEEQYQKALPLIKTQLKALIARDLWDMNEYFRVMNTTNESVQKALEIFRSGEYQKKLK

SEQ ID NO: 72 - protein, 34 aa, synthetic construct
MLGIAAMLASCSQNEELFTDKGNNTVQQMTFTLA

SEQ ID NO: 73 - protein, 30 aa, synthetic construct
MLVMFVWLTACNRDPHEGERGLTVAIDNTQ

SEQ ID NO: 74 - protein, 193 aa, synthetic construct
MKTITNLIFIALLFVTASLSAQEHGPVLGGSLGTLHEKVSVVSSNFEGYKLHYHIGYAYRQYWGNRKFAMDGIALFGSQGTDYQAFNSDSPKYSVNGKYLSIGTVFSYECFSNFRLGVGADVAWYVNRLGASTYNSKKNAIDIPIVARVSYTLKWLELQLSYKQGTCNLMNTSGVGKVTSRNIQFSVFMPIFK

SEQ ID NO: 75 - protein, 173 aa, synthetic construct
MSLLAGISFISCGNSSRAKVESEAAQTGEDFKSFLDKFTSSAAFQYTRVKFPLKTPVTLLADDGETEKTFPFTKEKWPLLDSETLKEERITQEEGGVYVSKFTLNEPAHKVFEAGYEESEIDLRVEFELLPDGKWYVVDCYTGWYGYDLPIAELKQTIQHVQEENAAFKELHP

SEQ ID NO: 76 - protein, 60 aa, synthetic construct
MDTEYVTLKNLEVLDKWVKTSRNQYKGTIRRSVWLSEAGTCSPSYEDDDLQDQAAGFAYG

SEQ ID NO: 77 - protein, 143 aa, synthetic construct
MKRIVFWMVALLLMSGVAMAQGNRQGGRQQMDPKTRAERMTERMVKEYSLNEDQKQQLQDVNLTWVQKMAANQGGRSKDNKAAKMTKEEREKKMAEMKKSREDYDAQLKKIMTKEQYDSYVKKQAEREKQMKEGRQNRQKRQG

SEQ ID NO: 78 - protein, 30 aa, synthetic construct
MKRIVFWMVALLLMSGVAMAQGNRQGGRQQ

SEQ ID NO: 79 - protein, 180 aa, synthetic construct
MKSFILMVITMVCAISLEAQNKSFYDFNVTTIDGKEFPLSSLKGKKVLVVNVASKCGLTPQYAKLQELYDKYKDKNFVIIGFPANNFMGQEPGSNEEIAQFCSLKYDVTFPMMAKISVKGKNMSPLYQWLTEKKLNGKEDAPVQWNFQKFMIDENGNWVGFVAPKESPLSEKIVTWIEQE

SEQ ID NO: 80 - protein, 294 aa, synthetic construct
MKSFIGAVIFICVALSANAQLLWKVSGNGLSSPSYIMGTHHLAPLSVKDGITGLQKAMDETQQVYGELKMSDIQSPATIQKMQKMMMIESDTTLTTLLSPEDYETANKFCKENLMMDLNMAPKIKPAFLLNNIAVVAYIKHIGNYNPQEQLDTYFQTQATQKGKKVDGLETPDFQFNLLYNGISLQRQAQLLMCTLNNIDKEVESLKRLTDAYMKQDLETMLKINEERKGNQCDALPTEEDALIYDRNKTWAQKLPAIMKAAPTFVAVGALHLPGNKGLLSLLKKQGYTVEAVK

SEQ ID NO: 81 - protein, 155 aa, synthetic construct
MKKLIALVVLLCGFMPVLWAADGCDQHLSREDFRNKQKAFIIEQAGLTKEEAAKFFPVYFELQEKKKKLNDESWSLMRQGKDDKTTEAQYEEIVAKVCDNRIAADRLDKSYLDRFKKILSNKKIFLVQRAEMRFHREMLKGMNRKDGGNDPNRKK

SEQ ID NO: 82 - protein, 354 aa, synthetic construct
MKKLIIATGLLMATSAYAQTEVLTGVTRGKDYGVVYALPKTQIEIEIKANKVNYKPGEFSKYADRYLRLTNVSAEPEEYWELNSVKVKPVGVPNSEATYFVKLKDKTVAPLMELTEEGLIKSINVPYSSNNAKNAANNAVAPQRKANPRDFLTEEILMASSTAKMAELVAKEIYNIRESKNALLRGQADNMPSDGAQLKIMLDNLNLQEEAMTEMFSGVRNKEEKTFTVRLTPDKEFDNEVAFRFSKKLGIVANNDLAGTPFYISLKDLKTVKIPQEDGKKKKEMEGIAYNVPGQAMVTLTDGKKKLYEGEIPVTQFGIIEYLAPVLFNKNSTIKVYFDPVTGGLLKVDREESK

SEQ ID NO: 83 - protein, 475 aa, synthetic construct
MKRKYILFIICSFFLGGVSAQTLEQARTLFTKGDYEQAKPVFQKYAKSQPSNGNYSYWYGVCCLKTGEPEEAVKYLETAVKRRVAGGQLYLGQAYNDTYRFEDAVNCFEEYIADLSKRKRSTEEAEALLEKSKADLRMLKGVEDVCIIDSFVVDKANFLSAYKISEESGKLFTFNEFFQTEGDHPGTVYETEIGNKIYYSEKGERGNLDIFSKNKLLNEWSNGRPLPGSINESGNANYPFVLSDGVTIYYATDGEGLGGYDIFVTRYNTNTDSYLTPENVGMPFNSPYNDYMYVIDEYNNLGWFASDRFQPEEKVCIYVFIPNSSKQTYNYEAMDPQQMIRLAQIHSLKETWKDENTVTEALQRLKEAINHKPQERRVVDFEFVIDDSTTYYQLSDFKSPKAKLLFQSYRQLEKDYLQQEDKLNGLRQQYASANQQGKEKLAPAILDLEKRILQMNEELDALGVSVRNAEKTKSK

SEQ ID NO: 84 - protein, 188 aa, synthetic construct
MLGGDFMSSEVSIGVNLKNKFIPTIEFGMGGTDTWNETGIHYKSKTAPFFRIGVDYNTMAKKKEKNSYLYVGLRYAFSSFKYDVSTLPVDDPIWGGSIGNPSLEDDYWGGSVPFSHLGMKGSMQWFELVVGVKVRIYKNFNMGWSVRMKYKTNASTNEYANPWYVPGYGKFKSNNMGITYSLIYKLPL

SEQ ID NO: 85 - protein, 168 aa, synthetic construct
MRKIAVLSLFLCLVAVCRAQYIGSQYMNVKLGYIVDESQVQGTFGFGKVYKAFKVGANLNYRNLNRDLVKANTVTVGPELAYYALKGRKFSLLGIAAGTIGYQKAKAKSDLVYLEKSKAFVYGYEVGIRPEMLLSPKVALFAEYRFEMLFNSILRNNNHVGLGCVIYL

SEQ ID NO: 86 - protein, 27 aa, synthetic construct
MKKRKILSLIAFLCISFIANAQQKLTS

SEQ ID NO: 87 - protein, 237 aa, synthetic construct
ASTPGGAPVPYPDPLEPAGENSLAKNVLSGGKGNDKLYGSEGADLLDGGEGNDLLKGGYGNDIYRYLSGYGHHIIDDEGGKDDKLSLADIDFRDVAFKREGNDLIMYKAEGNVLSIGHKNGITFKNWFEKESDDLSNHQIEQIFDKDGRVITPDSLKKAFEYQQSNNKVSYVYGHDASTYGSQDNLNPLINEISKIISAAGNFDVKEERSAASLLQLSGNASDFSYGRNSITLTASA

SEQ ID NO: 88 - protein, 22 aa, synthetic construct
MKLLKVAAIAAIVFSGSALAGV

SEQ ID NO: 89 - DNA, 591 bp, synthetic construct
  source 1..591: mol_type = other DNA
atgataaaatatataatgagaaagattgtattatcactatttgcattatgctgtttttcggcagtaatggcacaacagaagacacgcagtctgtctgtagaattacttggtgcccaaactattgtgggcatcaactacgacagcagattcgatgtaaactcaggtttaggctaccgtgtgggcattggctatggctatggagacaattctggttggcccgaccagaaaatcaatggcgttggtgttccgttggagctaaattatctgctcggaaagaagaatagcaagttggagctgggtttcggtgcgagtcttggtgtatatcaggtaaaggaaacaattggatacgtaaaagatacaggatggtatccaggtggggagaatacagacgaaacttctcctaaaactgaattttacacttcatcaaaaactcaattcggttatttcttcttcggcgacatcggctaccgctatcagcgccccaacggcttcatgtttcgtgtaggcgtttcgccgtcgttcaactttggcgacaagtacggactcaacaaggctgctttctttccgtacataggcttcggttggtcgttc

SEQ ID NO: 90 - DNA, 2460 bp, synthetic construct
  source 1..2460: mol_type = other DNA
atgatgaaaaaaacaattctgcttacatctattattgcgatagcaatagtaagcatgttatccagttgcgtagattccgagaaggatctatatgatccgtcttatcagacagccaatccaatgggagacggttttgctgcaccggatggatttgactggaatatgactacaactagtattctaaacatagaaattgatgatgaactttacaaccaaatagaaatattggacgcaaatccattttcaacgtctgattaccatatacttgccaaaggagttgccaagaaaggtcaagctttttcacaagaaataaattatacagaaggaactaattacctatatattcgtaagacagactctagaagcagagtgtctattagtacatgggatgtatctaaaaataaagaatttgtaggttcaaggactacaagagttgcaaaagcgaccatcggaagctataatattccagaaaaatatcccgaagaaacttatgataccacaggagctattgaacttacaggtaatactaattggaatcaatcaaatcatcatttggaagcaggcaaaagctatatcataaaaaacaaatttaatggtgaaatcaaccataccagtggatatttgaatggcggaagatttaccatatttgtagaaggtgaatggacaccctcgcaaaaccaaatacaaagtgcagatattattattttgaagggtggaaaaatcaacaccgattcattcacgagttttcttattgctgacaactcaatattaacgattcaatcaggcggaagtcttattggcaataacatcaatcttgctgccataggagtattactgaaaaattttggaacaattagtgtcaatagtatgaaggacttgaatacgacgtctattttatacaatgctcctaaagccacaattaatgttacaggaaaatctgttgcttcttgggaacagtccgtatttaccaaaggagctatttataactttggagaattgactattcaagaaggtgcattaaagtttaattctcaagatgcaacatgctatttttataatggaactgaagcaacaattaatactccaactttcattataggtgggattggtgtaaatgatggaactgttaatgctcaaaaaataagtaatgataatggcggtaaccctacttttactaataattgcagtctttatgcccaaaattctttcgaattcggaggaacaagcggaactatcataatgaataaaggtatattggctggtggcgtagaaaatggtacttttatagctattccatcttttaaatgcggtaatagcggaagcacttttgaactgaataatggttctatgataaaagcagagataatggatattcctaatgtcacttttaaagctgccggaactcgttcattaataaaatccacaaaaagcatatctactggttggacaactaagttcaatggtaatttggatattgaatgcccggaaggggaattcgccaaaggggtacctgccaataaccctaattacattatggaaaattcagtagaattatatatcccgaatggttccaaaacaatcattacttcatgtggtgaactttcagaaatacctgaccctacccctgacccggaagatcctgaattccctattgaagtggaagacaacaaagattatacttacctcttcgaagaccaatggccactatatggagattatgatatgaatgatatcgttctgaccattcagaaaaggaaaatatatactaataaagagaataaagtaacaaaattcgagttaagtattgacttatccgcagccggagcaaccaaaagcatcggagcagctattatgctggacaatgtacccgccaatgccattacacagtcagttgaattcagtgacaatactcttgccaagaatttcaatctaaataataacaatatcgaaagtggtcaggattatgctgtgattcctctgtttgatgatgctcataaagtattgggaagagatcggtatgagcagattaatactgtctcagactatgcaggcaataccaaacctaagaatatcagtttctccattaccttcaataatccgacaatatcagcagatgcttttaatgtcaacaatttgaatgtctttatcatcgtagacggcaacagaaacccacgtaaggaaatacatgttgcaggctatcaaccaaccaaactcgcaaacaccgatctgtttggcggaaacaacgataacagtcatcatggcagcaaaaaatattatatcagtaaagagaatctggcatggggtatcatggttccttccaatttcaaatggcctttagagtatgtaaatatcaaaactgcttattcacaattcagcgactgggtaacgagcggtggtacagaaaatgaaaaatggtggaatgattttgatgtaaacaaggtattccaaacaaataaaaac

SEQ ID NO: 91 - DNA, 1221 bp, synthetic construct
  source 1..1221: mol_type = other DNA
atgagaaacaagaccctgagatggaaaaccatcgctctttgttccatcatgttttgcctgccgaagactcctttggcagggcaggagtcttttggttttatccatcgtttgggaatggaagcccgtccgcaatatgtatttcctaccaaccccttcctgcaaggagagaacgaacgctggaaaccgattcagacatcgtttgccgcccacttgaaatactcgttcaagtttcgccccaatacttgtgccgaccgtatttacggtggagcttatcagggtatcggtgtgtcccttaccacctttggtgataaaaaacagttgggcgatcctttctcattctatgtgtttcaaggagcacgaattgcccgtttctctccccgtgcctcgctcaattatgagtggaatttcggtttgtccgccggatggaagccatacgacaattattataattcctataatggtgcggtcggttcacggatgaatgcttatatcaatgccggagtttacataaactgggcgttttcccgttacttcgatctgattgtcggaggcgatttcacgcacttctccaatggtaacactaaattcccgaatgcgggaatcaatactgccggagccaagatcggactcgtctacaatttcaatcgtacggaagaagacctctcgaaatcactctatcagcctgtcaccacccgttttccccggcatatcagttatgatgtcgtgctgttcgggtcatggcgtcgaaagggagtgtgggtaggagagaagcagatcgcatctcccaatgcgtatcccgttgccggattcaattttgccccgatgtataatctgggttataaatttcgcgtgggagcttcactggacggtgtctatgatggcagtgccaatgtatatagggaagatgtaattgtggaatatgggagcagcagttctaaacgtgagttcctgaaaccgggcattcagcatcaactggccttgggactttcgggacgtgcggaatatgtgatgccttattttaccattggcgtcggtatgggagccaacgtgctcagccgtggcgatatgagaggcttttaccagattcttgcattgaaaatagccgttacccgcagttcctttcttcacatcggatacaatttgcagaacttccagacgcccaactatctgatgctcggactcggtttccgtttccacaataaatatcctaaggtacgtcat

SEQ ID NO: 92 - DNA, 735 bp, synthetic construct
  source 1..735: mol_type = other DNA
atgaatatactgcgcaacataattcttttcggttggatggcgatagcttgcacagctttcgcccaagatcgtaacgcagataaagtggaacgtcccaacacaaagaaaatcaactttggtatcaaagccggatttaattcttccatgtttatggtatccgaattgaaaatcaaagacgtaacgatcgacgaagtacaaaataattataagatcggctattttggcgcgatcttcatgcgctttaatataaagaagcatttcattcagccggaagcatcctataatgtcagcaaaggcgagatcacctttgacaaactcggttcgcaacaccctgccatcgaacccgattatgcttcggtacagtccgtacttcacagtatcgacttccctattctctacggatataacgtagtgaaaaaaggaccatacggaatgtctatctttgccggtccaaaactccggtatctatggggaaagcagaacgaaatcactttcacaaacttcgaccagaaagggattcatgaaaagctgtatccgctcaatgtcagcgtcgtgatcggtgtcggcgtcaatatctcccgcatctttttcgacttccgctacgaacaggggataggaaatatctccaaatccatcgtttatgacaatatcaattcagacggcagcacgggagtcagccctatcattttccgccgccgcgacagcgcattaagtttctcgttcggattcattctt

SEQ ID NO: 93 - DNA, 801 bp, synthetic construct
  source 1..801: mol_type = other DNA
atgatgttgcgattctctgttattacttatacttttggacgattaacaactaaaaaacacaataaagttatgaagaaattgatttttttatttgcattttgccttactgtgactaacattttcgctcaaactgaccctagccaattgaaaaaagaaggaagcgacgcttttaatgccaagaactatcccgtagcttatgctaaattcagtgagtatctgaaacaaacgaataaccaggattctgcaatcgcttattactgtggtatggctgcagatgaagtgaaaaagtatgcagaagctgtgactttctttgatattgctattcaaaagaaattcaatataggtaatgcttatgcccgtaaagcgctggcactggatgctatgaagaagactgacgagtatgttgctaccttggaagaaggtctgaaagtagacccggacaataaaacaatgattaagaactatggtctccactacctgaaagcaggtatcgctgcgcagaaagccggaaaagtggaagaagcagaagaatgctttaagaaagtgattcctttggatcataaaacatataaaaccaatgctttgtatagcctgggcgttttatgctataacgacggtgcgaatatcctgaaaaaggctgctcctctggcaaactctgatgcagacaaatatgctgcagaaaaggaaaaggcagatggcagatttaaagaagctataggttacttggaagaagctaccaaagtttctccggaagatacaaagtctaaaactatgctttctcaagtgcaggcagctatgaag

SEQ ID NO: 94 - DNA, 789 bp, synthetic construct
  source 1..789: mol_type = other DNA
atgagaaattcaaaggttacatctatgttggtagtgctaatgtttctgttggtcacaggaatacaggcgcagaccgttactcccagtaagaaatatatcacaaaagaactgaataacgtaagcaacttcagctcaatcagcgtattaggcagtcctgatgttgagtatcgtcagagtaatggttctaaaacgacagtttctatatacggttcggataatttggtagacttgctggaagtttctacagtaaatggagtattgcaggtgaatatcaaaaagggtgtcaagattcttagtggagagcgtcgtttgaaagtgatagcctcttccccttcattgaatcaggtaaatattaaaggttcggcagatgtatatctgaaaggtgcaatcaaagggaatgacctgaatttgaatatagccggttccggagatatcgaagcggagaatctgcaatatactaatatatttgctttggtgaaaggaagcggggacattgatttgaaaaatgtgaaagcgactactgtgatgtcagaagttaatggttccggtgatatcaatattaaaggttctgcacagaaggcgacactgacagtaaatggttccggagatattagtgctgaaaaactggctgcaactaatgtcgttgctacagttgcaggttcgggtgatatagtatgctacgcttccagacagttggatgcgagagttagtggtagtggtgatattgaatataaaggaagcccttcagttgtaaataagcaggggaagaaaaattcgattaccggtaag

SEQ ID NO: 95 - DNA, 393 bp, synthetic construct
  source 1..393: mol_type = other DNA
atgaaaaagatactttttttaatgaccttgttagtggtaggagtaagtttcgcttttgcccagactaacgctgatatcaagtttgacaaaacaacacacgactttggaaaattctcagaaaacagtccggtagtaagctgcgtattcactttcactaatatcggtgatgctcctttagtaattcatcaggcggtagcgtcttgcggatgcacagtgcccgaatataccaaagaaccgatccagccgggaaagaaaggtacaatcaaagtaacttacaacggaacaggtaaatatccgggacacttcaaaaagtcgattaccctgcgtaccaacgcaaaaaccgaaatggtacgcctctatatcgaaggcgatatgaccccgaaagacgagaag

SEQ ID NO: 96 - DNA, 1152 bp, synthetic construct
  source 1..1152: mol_type = other DNA
atgagccgccggagattccttaaaatggcggcattgacaggagctgccatgtgtgtcgggcctaccttgaacaaggtaacggcagcagaacgggtatggatggggaaacaacaagttgcaaacaacatccctgcaggcatggcggcggtgcgcactcggcgaacattgggacacggaaatacggcattcactgtttctgcaatgggatatggctgtatgggcttgaaccataaccgcagccagtacccgagcagggaaaaagaaatcgccctggtgcatgaagctgttgaacgtggggtgacccttttcgatacagccgaaagctacggctaccacatcaatgaaaaattggtcggcgaagccttgaaaggatataccgaccgggtatttgtgtcatcgaagttcggacataagtttgtaaacggtgtgcagattaaaaccgaagaggacagcactccggccaacatccgccgggtttgtgaaaactcgttgcgcaatctcggagttgaaacattgggcatgttctaccagcaccgcatagacccgaacacaccgattgaagcggtggctgaaacatgcagcaaactcatcaaggaaggtaaaatcctgcattggggcatgtgtgaagtaaacgttgataccatccgccgtgcgcacaagatatgtccggttacggcaatccaaagcgaatatcacttgatgcaccgcatggtagagacgaacggagtgttggagctgtgcgaggatttgggcatcggtttcgttccatacagtccgctgaaccgtggttttttgggcggtatgatcaatgaatataccaagtttgatgttaccaacgacaaccggcagaccttgccacgcttccagcccgaagccatacgcgccaactatcgtattgtggaagttcttaacgctttcggccgtacaagaggcattaccccggcacagatagctttggcctggctgatgaacaagaagccttttatcgtgcctattccgggcaccaccaaactctctcatctagaagagaaccttcgtgcctgcgacatccgtttcacggcagaagagatagaagaactggaaacagccgtagctgccattcctgtggtgggtagccgctacgatgccttgcaggagtctaaaatcccaaaa

SEQ ID NO: 97 - DNA, 810 bp, synthetic construct
  source 1..810: mol_type = other DNA
atgaaagtgagaagaaatttattaatcgccctttcccttctgtctctcagcgccaacgcacagcgcatcaaaggcagcgacactgtattgcctgttgcccaacaaacagcagagcgatttatgaatctgcagccggatgcacgggtcacagtcaccggcggaggaaccggtgtcggtatctctgcactgatggacaatacgacggatatcgccatggcttcccgtccaatcaaattcagcgaaaagatgaaaaccaaggaagccggacaagaggtggacgaagtcatcgtagcctacgatgcccttgccgtagtggtgcatccttccaatccggtgaaacagctcacccgccagcaactggaagacatcttccggggaaaaatcaccaactggaaacaggtaggcggcgacgaccgcaaaatagttgtctactcccgcgaaacctcttcgggtacttatgagttcttcaaggaaaacgtgctgaaaaataaaaattatatggcaggcagcctttccatgcccgccacaggtgccatcatccaatccgtcagccagacgaaaggagctatcggatatgtcggcctggcatacgtatctccgcgcaccaagacgctttccgtatcatatgacggaagccactatgccgccccgacggtggagaacgcaacgaacaaaacttatcccatcgtccgccccctttattactactataatgtgaagaataaagaacaggtagctcctctgattgacttcattctttcacccgaagggcaggagattataaaaaagagcggttatattccagtaaaa

SEQ ID NO: 98 - DNA, 99 bp, synthetic construct
  source 1..99: mol_type = other DNA
atggctatagcagccactcttttggcttcctgcaacaaagatgaggaagaaacggaaattcaaggattcaaagtgctggaatatcgtccggcaccaggg

SEQ ID NO: 99 - DNA, 390 bp, synthetic construct
  source 1..390: mol_type = other DNA
atgaagaaacgagtgcttttaggaatggcctctttgcttctttctgtctcattgctgatggcgcaggagataccggcaggagtgattacggcatttaagcggggaagctcgcaggaactgagtaagtatatgggagacaaggtaaacctcgttctccaaggcagctcggcaaacgttgacaagaagaaagcgacagcaatgatgcaagagtttttcacggaaaacaaggtcaacgcatttgacgtgaatcatcagggaaaacgggatgaatccagttttgtcataggtacgctgacgactacaaaagggaaattccgtgtgaactgcttcctgaaaaaagtgcagactcaatatttaatacaccaaataagaattgacaagatcaatgaa

SEQ ID NO: 100 - DNA, 816 bp, synthetic construct
  source 1..816: mol_type = other DNA
atgataagaattattttaattttatttcttagctttccggttgccctgtgtgcgcagacctatcagcaattatcagaaaaggcaattgaatatatcgaaatggacagtctttccaaagccgaagaactactgcttcaggctttgaaactggagccgaagaatgcaaagaatgctatgcttttttccaatctgggattggtgcagcgtcggttgggtgaatatgacaaggcactcgaatcttattcttttgcgctgaactttgctccattggcagtacctattctgctggaccgagctgcgattaatctggaaatggggaatacagaccgtgcttataccgattattgtcaggtacttgacgaagataaacagaataaagaagcgctgttgatgcgggcatatatctatgtgctccggagggattaccccgcggcacgaatcgattataatcgcttgttggagattgacccgcaaagttatagcggtcggcttgggctggctaccttggaacagaaagaaggaaaattcaaggaagcgctcgaaattcttaataagatgattacggcaactcccgaagacgcgactctttacattgcccgtgcagatgtggaacgggagatgaagcatgaagatctggctttggtcgatttggaggaagctattcgcctgaatccttcttcggcggatgcctatttgttgcgtggcaatatttatctgacacagaaaaagaaagcattggcaaagatggactttgaaaaagctatttcgctgggcatacctcctgctgatttgcacgagcagctgaaacaatgtaaa

SEQ ID NO: 101 - DNA, 96 bp, synthetic construct
  source 1..96: mol_type = other DNA
atgatgacaggacttacattactgagttgcagcactgaaaacgatgaattcaagaaggaactgcctccaacggagcaaccatctgaacccacagga

SEQ ID NO: 102 - DNA, 912 bp, synthetic construct
  source 1..912: mol_type = other DNA
atgaaactccaaataaatctatgtgcgctggctcttcttttaatgattccgtttacggccatggcacagtctaaaacaaaagcgaaagctaaaaaagaagtagcgattcaactctattctgtcagggatatattgaataaggtggacaataaggacggcaaatgtgacgccgcttatattactcttctgaagaacttggcgaaaatgggatataccagtgtggaagccgccaattataataatggaaagttttacgacagaactcccgatcaatttaaaaaagatgttgaatctgccgggctgaaggttttatcctcacattgcacaagaggattgtcgaaagaagaattagcttccggtgatttttcaagttcacttcaatggtgggaccagtgtattgctgatcataaagctgccggtatgagttacattgtagctccttggatggatgtgcctaaaacacttaaggagttggatacatattgtgcttattacaatgaaataggcaaacgttgtaaacagcaggggatgtcgttcggataccataatcatgctcatgaatttcagaaggtagaagataaagtgatgtatgactacatgattgagcataccaatcctgaatatgttttcttccagatggatgtttattgggtagttcgcgggcaaaacagtccggtagattattttaataaataccccggtcgttttaaaatgttccatatcaaggatcacagagaaatcgggcagagcggaatggttggtttcgacgctattttcaagaatgcaaagacggctggtgtgaagcatcttgttgcagagatagaaagctacagcatgcccgtagagaaaagtgtcgaggtaagtctggattatttgctggatgctccttttgttaaaagctcatacgctaaa

SEQ ID NO: 103 - DNA, 321 bp, synthetic construct
  source 1..321: mol_type = other DNA
atgacacgatcaggaaatactcggaatgggaaatatatgggagaggataaaaggagatatcatactataacgaatatattacaatataatctgatccctgatttttttactatatttgcaaagtattgtataacatttaaaaatatgaccatggaaaaaacatttgcacaaatcagtgaattgtttgatcaattttcaaaagatgccagcctccaggtagaaaaaggaaacaaagctgccggaacccgtgcccgcaaagcatcactcgaacttgaaaaactcctcaaacgtttcagaaaagagtcactcgaagcctcgaag

SEQ ID NO: 104 - DNA, 1449 bp, synthetic construct
  source 1..1449: mol_type = other DNA
atgtttttagctggagcgtttatagcagttgggtcgctctatgcgcaatcatctgatgccgaatggcaggcaggagttgcaaaattgaaagaaaccattcagacgaatccggctcaggccaatgaggaagctgaacagttgataaaaggaaagaataaaaagaatgtggaacttctggtggctatcggcgaggtatatctgaaagccggaaaacttccggaagcacaggagtatgctgctcttgccaaaaaggtgaatggcaaatcggctcttgcctccgtactggagggagacatcgcctttgagcagaaaaacgccggtctggcatctcagaaatatgaagaagctatctatttcgatccgagttgcacagaagcatatctgagatatgcggatatttataaatcagccaatgctgctctggccattgaaaaactggaacagctgaaagcccaggaaccgtccaacacagccgtagataagaaacttgccgaaatctattatttgaagaatgattttagtaaagctgccgaagcttattcgcgttttgcgatgggaccgacagcgacagaagaggatttagtgaaatatgcatttgccctcttcctgaaccatgacttcgagaagtcacttgaagtggcgaatatgggattgaaaaagaatgcgcgtcacgcggcgttcaatcgcctggcaatgtataattatacagacctgaagcgctttgatgaagcaataaaagcggcggatgctttttttaccgaatcggacaaggctgattattcatatcttgactatatgtattacgggcatttgctcgaagctctgaagaaatatgatgaagctgtaggacaatatgagaaagccatacaattagatccgacaaagacggacttatacaaaaatatctcttctgcctatgaacagaagaatgattataaaaaagcaattagtgcttaccagaaatattatacatcccttgataaagaacatcagactccggatttacaattccagtttggcagactttattatggtgcaggtacgcagactgattcgctgacaatcaatgctgaagagcgcaagcaggctttgatggccgctgattctgttttccattccatcgcagaagcagcaccggacagctacctgggtaatttttggagggcacgtgccaattccgcactggaccccgaaacgactcttggactggcgaagcctttctatgaggaggtagccaccctgctggaaagcaagaatgatcctcattacaattcagctttgattgagtgctacagttatcttggatattactacttattggctattgaaaatccggcactgaaagcggaagcaatggcgaataaagaaaaatctaaggagtattggagcaaaattctggctatagactccactaatgctactgctaaaagggcattggatggcatcaaa

SEQ ID NO: 105 - DNA, 1587 bp, synthetic construct
  source 1..1587: mol_type = other DNA
atgatgatgaagaagattaaagtatacgccctcctggtttgtctgttggcaacgtctgctgcacaggcccaaagtttcggctctgccgccatgcgcaaactccaaatggcggaattcgcaatttccaacttctacgtagacaaagtagatgaagacaaactggtagaagaagctatcatcaagatgctggcacagctcgacccgcactccacttactcggatgccgaagaagtgaagaagatgaacgaaccgcttcaaggcaacttcgaaggcatcggcgtacagttccagatgatcgaggacaccctgctcgtcgtacaacccgtcagcaacggcccctccgagaaagtgggcatccttgccggcgaccgcatcattgccgtcaacgactccgccatcgcaggagtaaaaatgagcacggaagatatcatgaagcgcctgcgtggcccgaaaggttcgaaagtcaacctgaccatcgtccgccgtggagtacaagacccgctcgtgttcaccgtgaaaagagataagatacctatcctgagtctcgacgcttcctacatgattcagccaaagatcggatacatccgcatcaaccgctttggcgcaaccaccgccgaagaatttaagaaagcaatgaaagacctgcaaaagcaaggcatgaaagacctcatcctcgacctgcaaggcaacggcggaggatatctgaacgccgccatcgaccttgccaacgaattcctcggacagaaggaactgattgtttacaccgaaggccggaccgcacagcgcagcgagttcttcgccaaaggaaacggagagttccgtgacggccgcctcatcgtactggtagacgaatacaccgcttccgccagcgaaatcgtgagcggcgcagtgcaggattgggacagaggtatcatcgtaggccgacgttctttcggcaaaggactggtgcaacgtcctatcgacctgccggacggctccatgatccgactgaccattgcacggtactacacccctgccggtcgttgcatccaaaagccttatgacagctcgaccgattacaataaagacctgatagaccgcttcaaccacggagaactgatgaatgccgacagcatccacttccccgactcattgaaagtgcagaccaagaaactgaaacgtaccgtctacggcggaggtggcatcatgccggactacttcgttccgattgatacgacactctacacggactatcaccggaaactggtcggcaaaggagtcatcatcaaattcacaatgaaattcatcgaagaccaccgcaaggaactggccgataaatataagaagttcgacagcttcaacgaaaagtttgttattgacgacgatatgctcgccaccctccgcgaaatgggtgagaaagaaggggtgaagtttaacgaagagcagtatcagaaggcacttccgctgatcaagacacagctcaaagcactgatagcccgtgacctctgggatatgaacgaatatttccgcgtcatgaatacaacgaacgaaagcgtgcagaaagcattggaaatcttccgttcgggagagtatcagaagaagttgaaa

SEQ ID NO: 106 - DNA, 102 bp, synthetic construct
  source 1..102: mol_type = other DNA
atgctgggcatcgcagcgatgctggcatcgtgcagtcagaacgaggagttgtttaccgacaagggaaacaacacagtacaacagatgactttcacactcgca

SEQ ID NO: 107 - DNA, 90 bp, synthetic construct
  source 1..90: mol_type = other DNA
atgctggtcatgttcgtgtggctgacagcctgcaaccgtgacccgcacgagggagagagggggttgaccgtagccatcgacaacacccaa

SEQ ID NO: 108 - DNA, 579 bp, synthetic construct
  source 1..579: mol_type = other DNA
atgaaaacaattactaatttaatatttatagctttgctatttgttacagcatcattatctgcacaggaacatggtcctgttttaggaggctctttaggaacattgcatgaaaaagtgagtgtggtttccagtaattttgagggatataaacttcattatcatattggatatgcttatcggcagtattggggtaatcgtaaatttgcaatggatggcattgctctttttggctcgcaggggactgattatcaagccttcaattcggatagtcctaagtatagtgtaaacggaaaatatctatcaatagggactgtgttcagttatgaatgtttttctaattttagacttggagtcggagcagatgtggcttggtatgtaaatcgattgggtgcatctacttataatagcaaaaaaaatgcgatcgatatacctattgtggcaagagttagttatacgctgaaatggcttgaactacaactatcttataaacaaggtacttgcaatctgatgaatactagtggtgttggaaaagtaacttcacgaaatattcaattttctgtttttatgccaatatttaaa

SEQ ID NO: 109 - DNA, 519 bp, synthetic construct
  source 1..519: mol_type = other DNA
atgtcattgctggcaggtatttcatttatttcctgtggcaatagctccagggcgaaagtagaaagtgaagcagcacagacaggagaagatttcaagtcgtttctggataagtttacttccagtgcggcgtttcagtatactcgtgtcaagtttccgttgaaaacgccggttactttattggcggatgacggggaaactgagaaaacgttcccttttacgaaggaaaagtggccgttgctggacagtgaaacgctgaaagaagaacgtatcactcaggaagagggcggtgtttatgtatctaagtttacgctgaacgaaccggcgcataaggtatttgaggcgggatacgaagaatcggagatcgacttacgtgtggagttcgaattgctgcccgatggaaaatggtatgtagtagactgttataccggctggtatggatatgatttgccgattgcagagctgaaacaaaccattcagcacgtgcaggaagagaatgcggcttttaaagaacttcaccct

SEQ ID NO: 110 - DNA, 180 bp, synthetic construct
  source 1..180: mol_type = other DNA
atggatacggaatatgtgaccttgaaaaatttggaagtgttggataagtgggtgaaaacatcccgaaatcaatataaaggaactatcagacgttctgtatggctttctgaagcgggcacatgttcgccatcttatgaagatgatgatttgcaggatcaggctgccggatttgcttatggt

SEQ ID NO: 111 - DNA, 429 bp, synthetic construct
  source 1..429: mol_type = other DNA
atgaaaagaatcgtattttggatggtagctttgctactgatgagtggtgtggcaatggcacaaggaaatcgccagggtggaagacaacagatggatccgaaaacccgtgcggaacgtatgacagagcgtatggtgaaagaatactctttgaatgaagatcaaaagcaacaattacaggatgtcaaccttacatgggtgcaaaagatggctgctaatcagggcggacgttcgaaagataataaagctgccaagatgactaaggaagagcgggaaaagaagatggcggaaatgaagaagtcccgtgaagattatgatgcccagttgaagaagattatgactaaagaacaatacgatagctatgtgaaaaaacaggctgaacgtgagaaacagatgaaagaagggcgtcagaaccggcaaaaaagacaaggg

SEQ ID NO: 112 - DNA, 90 bp, synthetic construct
  source 1..90: mol_type = other DNA
atgaaaagaatcgtattttggatggtagctttgctactgatgagtggtgtggcaatggcacaaggaaatcgccagggtggaagacaacag

SEQ ID NO: 113 - DNA, 540 bp, synthetic construct
  source 1..540: mol_type = other DNA
atgaagtcattcatcttaatggttataacaatggtatgtgcaatctctctggaagcgcagaataagtctttctacgacttcaatgtgaccacgattgatggtaaggaatttccgctttcttctttgaaaggaaagaaagtgctggtagtaaatgtagcctccaaatgcggactgacgccacagtatgccaaactgcaagagctatatgataaatataaggataagaattttgtaatcatcggctttcctgccaacaactttatgggacaggaaccgggaagcaatgaagagatcgctcagttctgctccttaaagtatgacgttacttttccgatgatggccaagatttccgtcaagggcaaaaacatgtcgccactttaccaatggctgacggaaaagaaactgaacggcaaagaagatgcgcccgtccagtggaattttcagaagtttatgattgacgagaacggaaactgggtcggctttgtggcccccaaagagagtccgctctcggaaaaaatcgttacttggatagagcaggag

SEQ ID NO: 114 - DNA, 882 bp, synthetic construct
  source 1..882: mol_type = other DNA
atgaaatcgtttattggagcagtcatatttatctgcgtagcgttaagcgcaaacgcacagcttttatggaaagtatctggtaacggacttagcagcccttcatacattatggggactcaccatctcgctccgttgagtgtcaaagacggcattaccggactacagaaagcgatggatgaaacacaacaggtatatggagaactgaaaatgtcggatatccagtctccggccaccatccagaagatgcagaaaatgatgatgatagaaagcgatacgactctgactaccctcctctcaccggaagattacgaaaccgctaataaattctgcaaggagaacctgatgatggacctcaacatggcacccaaaatcaaaccggctttcttactgaacaacatagcagtagtggcgtacatcaagcacatcggaaactataatccgcaagaacagctcgatacttatttccagactcaggcaacacaaaaaggaaaaaaagtggatggactggaaactccggatttccagtttaacctgctatacaatggaatttccctgcaacgccaagcccaactgctcatgtgtaccctcaataacatcgacaaagaggtggaaagcctcaaaagactgacagatgcctacatgaaacaagatttggaaaccatgttgaaaatcaacgaagagcgtaaaggaaatcaatgtgatgcacttcccacagaagaagatgccctaatttacgaccgcaataaaacatgggcacagaaactaccggctatcatgaaagccgctcctaccttcgttgcagtcggcgctttgcacctgccgggtaataaaggcttattaagcttactgaaaaaacagggatatacagtagaagcagtaaaa

SEQ ID NO: 115 - DNA, 465 bp, synthetic construct
  source 1..465: mol_type = other DNA
atgaagaaactaatcgctttagtcgttttgttatgtggctttatgcctgttctttgggctgccgatggatgtgaccagcatttgtcccgtgaggatttccggaataaacagaaagcgtttattattgagcaggccggattaaccaaagaagaggctgccaaattcttcccggtttattttgaactgcaggagaagaagaagaagctgaacgatgaatcgtggagtttaatgcgtcaaggtaaagacgacaaaacaacagaagcccagtacgaagaaattgtagcaaaggtatgtgacaaccgtatcgctgccgaccgtcttgataagtcttatctggaccggtttaaaaagatactttccaacaagaagatattccttgttcaacgcgcggagatgcgtttccatcgggaaatgctgaaaggcatgaaccgcaaggacggaggaaatgatccgaacaggaagaaa

SEQ ID NO: 116 - DNA, 1062 bp, synthetic construct
  source 1..1062: mol_type = other DNA
atgaaaaagttaatcatagcaacaggactactaatggcaacttccgcctatgcgcagaccgaagtgctgacaggagtgacgcgaggaaaagactacggagtagtgtacgccctccccaaaacccagatagaaatagaaataaaagcaaacaaagtcaactataagccgggtgaattcagcaaatacgccgaccgctacctgagactgaccaacgtctctgccgaacccgaagaatactgggaactgaacagtgtcaaagtgaaacctgtaggtgtaccaaacagcgaagctacttatttcgtcaaactgaaagacaaaaccgtagctcctcttatggaactgactgaagagggtcttatcaaatcaatcaacgtcccctacagcagtaacaatgcaaagaatgcagcaaacaatgccgttgctccccaacgaaaagccaatccccgcgactttctcacagaagaaattctgatggcaagttcaaccgccaagatggccgaactggtagcaaaggaaatctataatatccgcgaaagtaaaaacgcattattgcgcggacaagcagataatatgccttcggacggcgcacagctcaaaatcatgctcgacaacctgaatcttcaggaagaagccatgaccgagatgttttcgggagtccgcaataaagaagaaaagacatttactgtccgcctcactccggataaggaattcgacaatgaggtagctttccgcttttcaaagaagctgggtatagttgccaacaacgatttggcaggcactccattctacatcagtctgaaagacctgaaaacggtgaagattccgcaagaggacggaaaaaagaagaaagagatggaaggcattgcttataacgtaccgggacaggcaatggtgacgctgacggacggcaaaaagaaattgtatgaaggagaaatacctgttactcagttcggtatcatagaataccttgctccggtattattcaacaagaactcgaccatcaaagtttactttgatccggtgaccggtggactactgaaagtagatagagaagaaagcaaa

SEQ ID NO: 117 - DNA, 1425 bp, synthetic construct
  source 1..1425: mol_type = other DNA
atgaaaagaaaatatattctatttataatttgcagtttcttcctcggaggagtatctgcacaaacattggaacaggcacgcaccttatttacaaaaggtgactacgaacaagccaaacccgtatttcagaaatatgccaaatctcaaccatccaatggtaactacagctattggtacggtgtctgctgcctgaaaaccggagaaccggaagaagccgtgaagtatctcgaaaccgctgtaaaaagacgggtagccggtggacaactctatctgggacaagcttacaatgatacttatcgtttcgaagatgcagtcaactgctttgaagaatatatcgccgacctcagcaaacgcaaaagatcgacggaagaagctgaagctctattggaaaagagcaaagctgacctgcgtatgctcaaaggagtagaagacgtgtgcattattgacagtttcgtggtagataaagcaaatttcctgagcgcatacaagatcagtgaagagtctggcaagctatttacattcaacgaattcttccagaccgaaggagatcatccgggaacagtttatgaaacagaaatcggtaataaaatctattatagcgaaaagggagaaagaggcaatctggatatattctccaaaaataaattactgaatgaatggagcaacggtcgcccgttgcccggcagcatcaacgaatccggaaacgccaactacccgtttgttttatcggatggtgttactatttattatgcaaccgatggcgaaggactgggaggatatgatatttttgtcactcgttacaacacaaatacggactcttatctgacgccggaaaatgtgggaatgccgttcaattcaccttataatgattatatgtacgtcatcgacgaatacaataatctgggttggtttgcctccgaccgtttccagcccgaagagaaagtctgcatctatgtattcattcctaattcgtctaaacagacttataattatgaggcgatggacccgcagcagatgatccgtctggcacagattcattcgctgaaggaaacttggaaagatgaaaatacagtaacggaggcactgcaacgtctgaaagaggcaatcaatcataagccacaggaacgtcgtgtggttgatttcgagtttgtcatcgatgatagcacgacctattatcagttgagtgacttcaaatcacccaaagcaaagctactgttccaaagttaccggcaactggaaaaagattatctgcaacaggaagataaactgaacggtctgcgccagcaatatgcaagcgccaatcaacaaggaaaagaaaaacttgctcccgcaatccttgatctggaaaaaagaattctgcaaatgaatgaagagctggatgctcttggagtaagtgtgcgtaatgccgaaaaaacaaaatcaaaa

SEQ ID NO: 118 - DNA, 564 bp, synthetic construct
  source 1..564: mol_type = other DNA
atgctaggtggcgacttcatgagttccgaagtcagcataggcgtcaaccttaaaaacaagtttatcccgaccatcgagtttggtatgggaggaacagatacatggaatgaaacgggaatacactacaaaagtaagaccgccccattcttccggattggtgtagactataatacgatggcaaagaaaaaggagaaaaacagctatctatatgtaggtctccgctatgcgttcagttcattcaagtatgatgtatcgaccctgccggttgacgatccgatttggggaggaagtatcggaaatccgagtctggaagacgactactggggaggaagcgttcctttcagtcatttagggatgaaagggtctatgcagtggtttgaattagtggtaggtgtcaaagtacgcatttacaagaatttcaatatgggctggtctgtacgcatgaaatataagacaaatgcttcgaccaatgaatacgccaacccttggtatgtgcccggctacggaaaatttaaatcaaataacatgggaattacctattccctcatttacaaattacctctt

SEQ ID NO: 119 - DNA, 504 bp, synthetic construct
  source 1..504: mol_type = other DNA
atgagaaaaattgccgtattatcgctctttttgtgcctggtggccgtgtgcagggcacaatatatcggaagccagtatatgaacgtgaagctgggctatatcgtggatgaaagtcaggtgcaggggacgttcggttttggaaaggtgtacaaggctttcaaggtgggggcaaaccttaattaccggaatctaaaccgggatctggtaaaggcaaacaccgtgacggtcggcccggagctggcctattacgccctgaaaggacggaaattctcgctcctggggatcgcagccgggacgatcggctaccagaaagccaaggcgaaaagcgatcttgtgtacctggagaaaagcaaggctttcgtgtatgggtacgaggtcgggataaggccggaaatgttgctaagcccgaaagtggctctttttgccgaatacaggtttgaaatgttgtttaactccattctccggaataacaaccatgtcggtctggggtgcgtgatctattta

SEQ ID NO: 120 - DNA, 81 bp, synthetic construct
  source 1..81: mol_type = other DNA
atgaaaaagagaaagattttatcgctcatcgcattcctttgcatctcattcattgcgaatgcccaacagaaattaacctca

SEQ ID NO: 121 - DNA, 714 bp, synthetic construct
  source 1..714: mol_type = other DNA
gcttcgacgcccgggggtgcgccggtgccgtatccggatccgctggaaccggccggggaaaattctcttgctaaaaatgtattatccggtggaaaaggtaatgacaagttgtacggcagtgagggagcagacctgcttgatggcggagaagggaatgatcttctgaaaggtggatatggtaatgatatttatcgttatctttcaggatatggccatcatattattgacgatgaaggggggaaagacgataaactcagtttagctgatatagatttccgggacgttgcctttaagcgagaagggaatgacctcattatgtataaagctgaaggtaatgttctttctattggccacaaaaatggtattacatttaaaaactggtttgaaaaagagtcagatgatctctctaatcatcagatagagcagatttttgataaagacggcagggtaatcacaccagattctcttaaaaaagcatttgaatatcagcagagtaataacaaggtaagttatgtgtatggacatgatgcatcaacttatgggagccaggacaatcttaatccattaattaatgaaatcagcaaaatcatttcagctgcaggtaacttcgatgttaaggaggaaagatctgccgcttctttattgcagttgtccggtaatgccagtgatttttcatatggacggaactcaataactttgacagcatcagcataa

SEQ ID NO: 122 - DNA, 66 bp, synthetic construct
  source 1..66: mol_type = other DNA
atgaaacttttaaaagtagcagcaattgcagcaatcgtattctccggtagcgctctggcaggtgtt

SEQ ID NO: 123-125 - skipped (3 entries)

SEQ ID NO: 126 - protein, 8 aa, synthetic construct
DYKDDDDK

SEQ ID NO: 127 - protein, 10 aa, synthetic construct
YPYDVPDYAC

SEQ ID NO: 128 - protein, 11 aa, synthetic construct
EQKLISEEDLC

SEQ ID NO: 129 - protein, 14 aa, synthetic construct
GKPIPNPLLGLDST

SEQ ID NO: 130 - protein, 13 aa, synthetic construct
GAPVPYPDPLEPR

SEQ ID NO: 131 - protein, 11 aa, synthetic construct
YTDIEMNRLGK

SEQ ID NO: 132 - protein, 7 aa, synthetic construct
HHHHHHH

SEQ ID NO: 133 - protein, 12 aa, synthetic construct
QPELAPEDPEDC

SEQ ID NO: 134 - protein, 8 aa, synthetic construct
SGPTGHGR

SEQ ID NO: 135 - protein, 8 aa, synthetic construct
SGPTGMAR

SEQ ID NO: 136 - protein, 8 aa, synthetic construct
SGPTASPL

SEQ ID NO: 137 - protein, 8 aa, synthetic construct
SGPTTAPF

SEQ ID NO: 138 - protein, 8 aa, synthetic construct
SGPTAAPA

SEQ ID NO: 139 - protein, 20 aa, synthetic construct
WLYACSLAIAFGVLSFVTVS

SEQ ID NO: 140 - protein, 18 aa, synthetic construct
TILLTSIIAIAIVSMLSS

SEQ ID NO: 141 - protein, 16 aa, synthetic construct
IYTLLIAFCAAWSLHS

SEQ ID NO: 142 - protein, 20 aa, synthetic construct
FLSVILFGALMTVSTGTFVS

SEQ ID NO: 143 - protein, 17 aa, synthetic construct
FFYLSALSLGMMCSITA

SEQ ID NO: 144 - protein, 15 aa, synthetic construct
LYTGCLLLMALITGS

SEQ ID NO: 145 - protein, 16 aa, synthetic construct
MLRIIMILLGALLLTN

SEQ ID NO: 146 - protein, 13 aa, synthetic construct
GILFVLTAAFLAS

SEQ ID NO: 147 - protein, 14 aa, synthetic construct
TIVPIIIGTLLSGA

SEQ ID NO: 148 - DNA, 120 bp, synthetic construct
  source 1..120: mol_type = other DNA
caattgggctaccttttttttgttttgtttgcaatggttaatctattgttaaaatttaaagtttcacttgaactttcaaataatgttcttatatttgcagtgtcgaaagaaacaaagtag

SEQ ID NO: 149 - DNA, 36 bp, synthetic construct
  source 1..36: mol_type = other DNA
gactgatcgtccatcaatttaaaatttaaaataatg

SEQ ID NO: 150 - protein, 5 aa, synthetic construct
MRNLK

SEQ ID NO: 151 - protein, 4 aa, synthetic construct
MMKK

SEQ ID NO: 152 - protein, 7 aa, synthetic construct
MNYSCRK

SEQ ID NO: 153 - skipped

SEQ ID NO: 154 - protein, 6 aa, synthetic construct
CHDDDD

SEQ ID NO: 155 - protein, 6 aa, synthetic construct
CQQEEN

SEQ ID NO: 156 - protein, 6 aa, synthetic construct
CSNDEP

SEQ ID NO: 157 - protein, 6 aa, synthetic construct
CSGDFE

SEQ ID NO: 158 - protein, 6 aa, synthetic construct
CDSEKD

SEQ ID NO: 159 - protein, 6 aa, synthetic construct
CDNDDD

SEQ ID NO: 160 - protein, 6 aa, synthetic construct
CKDYDD

SEQ ID NO: 161 - protein, 6 aa, synthetic construct
CSDDDT

SEQ ID NO: 162 - protein, 6 aa, synthetic construct
CSEEEN

SEQ ID NO: 163 - protein, 25 aa, synthetic construct
MRNLKWLYACSLAIAFGVLSFVTVS

SEQ ID NO: 164 - protein, 22 aa, synthetic construct
MMKKTILLTSIIAIAIVSMLSS

SEQ ID NO: 165 - protein, 20 aa, synthetic construct
MKLRIYTLLIAFCAAWSLHS

SEQ ID NO: 166 - protein, 24 aa, synthetic construct
MNKKFLSVILFGALMTVSTGTFVS

SEQ ID NO: 167 - protein, 20 aa, synthetic construct
MKKFFYLSALSLGMMCSITA

SEQ ID NO: 168 - protein, 20 aa, synthetic construct
MRKEKLYTGCLLLMALITGS

SEQ ID NO: 169 - protein, 18 aa, synthetic construct
MKMLRIIMILLGALLLTN

SEQ ID NO: 170 - protein, 22 aa, synthetic construct
CHDDDDEPKQEPGEVIETPAPV

SEQ ID NO: 171 - protein, 22 aa, synthetic construct
CQQEENEGVASVDRVTITPIIT

SEQ ID NO: 172 - skipped

SEQ ID NO: 173 - protein, 24 aa, synthetic construct
CSGDFEQETGIVPSHSGQVSFLFG

SEQ ID NO: 174 - protein, 17 aa, synthetic construct
CDSEKDLYDPSYQTANP

SEQ ID NO: 175 - protein, 18 aa, synthetic construct
CDNDDDESIAVPTPLQEA

SEQ ID NO: 176 - protein, 23 aa, synthetic construct
CKDYDDDINNLQEQIDGQKNDLN

SEQ ID NO: 177 - protein, 22 aa, synthetic construct
CSDDDTTTIDAKNLDYTAENAS

SEQ ID NO: 178 - protein, 21 aa, synthetic construct
CSEEENPEVRPATKPAEPYTS

SEQ ID NO: 179 - protein, 14 aa, synthetic construct
  VARIANT 2: note = K, N, R, S, E, D, or G
  VARIANT 3: note = D, N, E, or K
  VARIANT 4: note = D or E
  VARIANT 5: note = D, N, E, or K
  VARIANT 6: note = T, D, N, E, or K
CXXXXXTTIDAKNL

SEQ ID NO: 180 - protein, 40 aa, synthetic construct
MDTEYVTLKNLEVLDKWVKTSRNQYKGTIRRSWWLSEAGT

SEQ ID NO: 181 - protein, 6 aa, synthetic construct
CNKDEE

SEQ ID NO: 182 - protein, 6 aa, synthetic construct
CSTEND

SEQ ID NO: 183 - protein, 6 aa, synthetic construct
CSQNEE

SEQ ID NO: 184 - protein, 6 aa, synthetic construct
CNRDPH

SEQ ID NO: 185 - protein, 6 aa, synthetic construct
CSPSYE

SEQ ID NO: 186 - protein, 9 aa, synthetic construct
AIAATLLAS

SEQ ID NO: 187 - protein, 8 aa, synthetic construct
MTGLTLLS

SEQ ID NO: 188 - protein, 9 aa, synthetic construct
LGIAAMLAS

SEQ ID NO: 189 - protein, 9 aa, synthetic construct
LVMFVWLTA

SEQ ID NO: 190 - protein, 23 aa, synthetic construct
MMKKGILFVLTAAFLASCQQEEN

SEQ ID NO: 191 - protein, 15 aa, synthetic construct
MAIATLLASCNKDEE

SEQ ID NO: 192 - protein, 15 aa, synthetic construct
MMTGLTLLSCSTEND

SEQ ID NO: 193 - protein, 16 aa, synthetic construct
MLGIAAMLASCSQNEE

SEQ ID NO: 194 - protein, 16 aa, synthetic construct
MLVMFVWLTACNRDPH

SEQ ID NO: 195 - protein, 46 aa, synthetic construct
MDTEYVTLKNLEVLDKWVKTSRNQYKGTIRRSVWLSEAGTCSPSYE

SEQ ID NO: 196 - DNA, 168 bp, synthetic construct
  source 1..168: mol_type = other DNA
tttgcacccgctttccaagagaagaaagccttgttaaattgacttagtgtaaaagcgcagtactgcttgaccataagaacaaaaaaatctctatcactgatagggataaagtttggaagataaagctaaaagttcttatctttgcagtctccctatcagtgatagaga

SEQ ID NO: 197 - DNA, 199 bp, synthetic construct
  source 1..199: mol_type = other DNA
ttttgcacccgctttccaagagaagaaagccttgttaaattgacttagtgtaaaagcgcagtactgcttgaccataagaacaaaaaaatctctatcactgatagggataaagtttggaagataaagctaaaagttcttatctttgcagtctccctatcagtgatagagacgaaataaagacatataaaagaaaagacac

SEQ ID NO: 198 - DNA, 1045 bp, synthetic construct
  source 1..1045: mol_type = other DNA
cttaacggctgacatgggaattcccctccaccgcggtggcttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatattcgtttaatatcataaataatttattttattttaaaatgcgcgggtgcaaaggtaagaggttttattttaactaccaaatgttttcggaagttttttcgcttttctttttctatcgtttctcagactctcttagcgaaagggaaagaaggtaaagaagaaaaacaaaacgccttttctttttttgcacccgctttccaagagaagaaagccttgttaaattgacttagtgtaaaagcgcagtactgcttgaccataagaacaaaaaaatctctatcactgatagggataaagtttggaagataaagctaaaagttcttatctttgcagtctccctatcagtgatagagacgaaataaagacatataaaagaaaagacac

SEQ ID NO: 199 - DNA, 300 bp, Bacteroides sp.
  source 1..300: mol_type = genomic DNA
tgatctggaagaagcaatgaaagctgctgttaagtctccgaatcaggtattgttcctgacaggtgtattcccatccggtaaacgcggatactttgcagttgatctgactcaggaataaattataaattaaggtaagaagattgtaggataagctaatgaaatagaaaaaggatgccgtcacacaacttgtcggcattcttttttgttttattagttgaaaatatagtgaaaaagttgcctaaatatgtatgttaacaaattatttgtcgtaactttgcactccaaatctgtttttaaaga

SEQ ID NO: 200 - DNA, 43 bp, synthetic construct
  source 1..43: mol_type = other DNA
cgcattttaaaataaaataaattatttatgatattaaacgaat

SEQ ID NO: 201 - DNA, 211 bp, synthetic construct
  source 1..211: mol_type = other DNA
tttgcacccgctttccaagagaagaaagccttgttaaattgacttagtgtaaaagcgcagtactgcttgaccataagaacaaaaaaatctctatcactgatagggataaagtttggaagataaagctaaaagttcttatctttgcagtctccctatcagtgatagagacgcattttaaaataaaataaattatttatgatattaaacgaat

SEQ ID NO: 202 - DNA, 36 bp, synthetic construct
  source 1..36: mol_type = other DNA
gactgatcggcgcgactcacgcgccgatcagtaatg

SEQ ID NO: 203 - DNA, 36 bp, synthetic construct
  source 1..36: mol_type = other DNA
gactgatcaggaagagtaaaaaatattaaaataatg

SEQ ID NO: 204 - DNA, 36 bp, synthetic construct
  source 1..36: mol_type = other DNA
gactgatctctggggtgaataaaatttataataatg

SEQ ID NO: 205 - DNA, 36 bp, synthetic construct
  source 1..36: mol_type = other DNA
gactgatcccccattctattaaattttagaataatg

SEQ ID NO: 206 - DNA, 36 bp, synthetic construct
  source 1..36: mol_type = other DNA
gactgatcggtgttagctttaaatattagaataatg

SEQ ID NO: 207 - DNA, 36 bp, synthetic construct
  source 1..36: mol_type = other DNA
gactgatctagcactcttaaaaaaattaaaataatg

SEQ ID NO: 208 - protein, 10 aa, synthetic construct
  VARIANT 1..10: note = This sequence may encompass 6-10 residues
HHHHHHHHHH

SEQ ID NO: 209 - DNA, 36 bp, synthetic construct
  source 1..36: mol_type = other DNA
gactgatcgtaatctttaaaaaaaataaaaataatg

SEQ ID NO: 210 - protein, 27 aa, synthetic construct
CSNDEPTGGKGHQQTYSVVLKGITVAG

SEQ ID NO: 211 - protein, 5 aa, synthetic construct
SEDEN

SEQ ID NO: 212 - protein, 5 aa, synthetic construct
NEDEN

SEQ ID NO: 213 - protein, 5 aa, synthetic construct
SEDEE

SEQ ID NO: 214 - protein, 5 aa, synthetic construct
SDDDN

SEQ ID NO: 215 - protein, 5 aa, synthetic construct
SDDEN

SEQ ID NO: 216 - protein, 5 aa, synthetic construct
SEEND

SEQ ID NO: 217 - protein, 5 aa, synthetic construct
DDDNN

SEQ ID NO: 218 - protein, 5 aa, synthetic construct
SDEKD

SEQ ID NO: 219 - protein, 5 aa, synthetic construct
NNEEN

SEQ ID NO: 220 - protein, 5 aa, synthetic construct
EKDDK

SEQ ID NO: 221 - protein, 5 aa, synthetic construct
SKDDK

SEQ ID NO: 222 - protein, 5 aa, synthetic construct
NDDKD

SEQ ID NO: 223 - protein, 5 aa, synthetic construct
GKDKD

SEQ ID NO: 224 - protein, 5 aa, synthetic construct
NKDNK

SEQ ID NO: 225 - protein, 5 aa, synthetic construct
GKENN

SEQ ID NO: 226 - protein, 5 aa, synthetic construct
KEEKK

SEQ ID NO: 227 - protein, 5 aa, synthetic construct
SKEKK

SEQ ID NO: 228 - protein, 5 aa, synthetic construct
NKDKK

SEQ ID NO: 229 - protein, 5 aa, synthetic construct
GKDKK

SEQ ID NO: 230 - protein, 5 aa, synthetic construct
KNDKK

SEQ ID NO: 231 - protein, 5 aa, synthetic construct
KKEKN

SEQ ID NO: 232 - protein, 5 aa, synthetic construct
KKEKK

SEQ ID NO: 233 - protein, 5 aa, synthetic construct
RKDKK

SEQ ID NO: 234 - protein, 5 aa, synthetic construct
KKDKK

SEQ ID NO: 235 - protein, 5 aa, synthetic construct
KKKKK